(19) United States
(12) Patent Application Publication
(10) Pub. No.: US 2017/0240570 A1
(43) Pub. Date: Aug. 24, 2017

(54) NOVEL SMALL MOLECULE ANTICANCER AGENTS

(71) Applicant: UNIVERSITY OF FLORIDA RESEARCH FOUNDATION, Gainesville, FL (US)

(72) Inventors: Stephen Jahn, Gainesville, FL (US); Ronald K. Castellano, Gainesville, FL (US); Renan B. Ferreira, Gainesville, FL (US); Brian Keith Law, Gainesville, FL (US)

(21) Appl. No.: 15/519,433

(22) PCT Filed: Oct. 16, 2015
(86) PCT No.: PCT/US2015/056101

§ 371 Date: Apr. 14, 2017

Related U.S. Application Data
(60) Provisional application No. 62/065,467, filed on Oct. 17, 2014.

(51) Int. Cl. C07F 5/02; C07D 493/10; C07D 311/16; C07D 405/14; C07D 211/62; C07D 311/82; C07D 491/052; C07D 311/90; C07D 491/147; C07C 323/65; C07D 249/04; C07D 491/22; C07D 495/04; C07D 401/14

(52) U.S. Cl. CPC C07F 5/022; C07D 495/04; C07D 493/10; C07D 311/16; C07D 405/14; C07D 211/62; C07D 401/14; C07D 491/052; C07D 311/90; C07D 491/147; C07C 323/65; C07D 249/04; C07D 491/22; C07D 311/82

(57) ABSTRACT

The invention relates to methods of treating cell proliferative disorders. The invention further relates to pharmaceutical compositions for treating cell proliferative disorders, especially cancer.

### CROSS-REFERENCE TO RELATED APPLICATIONS

This application claims the benefit of U.S. Provisional Patent Application No. 62/065,467 filed Oct. 17, 2014, the contents of which are incorporated herein by reference in their entirety.

### BACKGROUND OF THE INVENTION

The Epidermal Growth Factor Receptor (EGFR) family members EGFR, Human Epidermal growth factor Receptor-2 (HER2), and Human Epidermal growth factor Receptor-3 (HER3) are well established as proto-oncogenes that play key roles in the initiation and progression of human cancers [Arslan, M. A., Kutuk, O., and Basaga, H. (2006) Protein kinases as drug targets in cancer Curr Cancer Drug Targets 6, 623-634; Yan, M., Parker, B. A., Schwab, R., and Kurzrock, R. (2014) HER2 aberrations in cancer: Implications for therapy Cancer Treat Rev 40, 770-780; Foley, J., Nickerson, N. K., Nam, S., Allen, K. T., Gilmore, J. L., Nephew, K. P., and Riese, D. J., 2nd. (2010) EGFR signaling in breast cancer: bad to the bone Semin Cell Dev Biol 21, 951-960]. EGFR is frequently mutationally activated in lung cancer and is the target of the FDA-approved drugs Cetuximab, Panitumumab, and Erlotinib. Although EGFR is rarely mutated in breast cancers, the wild type protein is frequently overexpressed in breast tumors, and EGFR has been suggested to be a therapeutic target in triple-negative (Estrogen Receptor-, Progesterone Receptor-, and HER2-negative) breast cancers [Park, H. S., Jang, M. H., Kim, E. J., Kim, H. J., Lee, H. J., Kim, Y. J., Kim, J. H., Kang, E., Kim, S. W., Kim, I. A., and Park, S. Y. (2014) High EGFR gene copy number predicts poor outcome in triple-negative breast cancer Mod Pathol].

HER2 is a transmembrane tyrosine kinase overexpressed in approximately 20-25% of human breast tumors [Gori S, Montemurro F, Spazzapan S, Metro G, Foglietta J, et al. (2012) Retreatment with trastuzumab-based therapy after disease progression following lapatinib in HER2-positive metastatic breast cancer. Ann Oncol 23: 1436-1441], usually as a result of gene amplification. HER2 has no known ligands and signals by forming heterodimers with the ligand-dependent receptor tyrosine kinases and HER2 family members EGFR and HER3. HER2 drives mammary tumorigenesis by activating several pathways that promote cell proliferation and survival including the PI3K/Akt/mTORC1 and Ras/Raf/MEK/Erk cascades.

Breast tumors are screened for expression of HER2 and patients with HER2-positive tumors are treated with chemotherapy combined with either the HER2-specific monoclonal antibody Trastuzumab, Trastuzumab+Pertuzumab, the HER2/EGFR tyrosine kinase activity inhibitor Lapatinib, or Trastuzumab+Lapatinib [Baselga J, Tripathy D, Mendelsohn J, Baughman S, Benz C C, et al. (1996) Phase II study of weekly intravenous recombinant humanized anti-p185HER2 monoclonal antibody in patients with HER2/neu-overexpressing metastatic breast cancer. J Clin Oncol 14:737-744; Vogel C L, Cobleigh M A, Tripathy D, Gutheil J C, Harris L N, et al. (2002) Efficacy and safety of trastuzumab as a single agent in first-line treatment of HER2-overexpressing metastatic breast cancer. J Clin Oncol 20: 719-726]. While these agents are effective in treating HER2-positive cancers, tumor resistance is a common problem. In fact, primary resistance to Trastuzumab as a monotherapy against metastatic breast cancer has been observed in 66-88% of patients [Cobleigh M A, Vogel C L, Tripathy D, Robert N J, Scholl S, et al. (1999) Multinational study of the efficacy and safety of humanized anti-HER2 monoclonal antibody in women who have HER2-overexpressing metastatic breast cancer that has progressed after chemotherapy for metastatic disease. J Clin Oncol 17: 2639-2648; Slamon D J, Leyland-Jones B, Shak S, Fuchs H, Paton V, et al. (2001) Use of chemotherapy plus a monoclonal antibody against HER2 for metastatic breast cancer that overexpresses HER2. N Engl J Med 344: 783-792; Piccart-Gebhart M J, Procter M, Leyland-Jones B, Goldhirsch A, Untch M, et al. (2005) Trastuzumab after adjuvant chemotherapy in HER2-positive breast cancer. N Engl J Med 353: 1659-1672]. While combining Trastuzumab with the taxanes Paclitaxel or Docetaxel improves patient outcomes [1], 15% of patients fail this therapy as well [Gayle S S, Castellino R C, Buss M C, Nahta R (2013) MEK inhibition increases lapatinib sensitivity via modulation of FOXM1. Curr Med Chem 20: 2486-2499]. Likewise, resistance to Lapatinib is a significant problem and investigating the mechanisms responsible for resistance to Trastuzumab and Lapatinib are areas of intense research [Wetterskog D, Shiu K K, Chong I, Meijer T, Mackay A, et al. (2014) Identification of novel determinants of resistance to lapatinib in ERBB2-amplified cancers. Oncogene 33: 966-976; Fabi A, Merola R, Ferretti G, Di Benedetto A, Antoniani B, et al. (2013) Epidermal growth factor receptor gene copy number may predict lapatinib sensitivity in HER2-positive metastatic breast cancer. Expert Opin Pharmacother 14: 699-706; Blumenthal G M, Scher N S, Cortazar P, Chattopadhyay S, Tang S, et al. (2013) First FDA approval of dual anti-HER2 regimen: pertuzumab in combination with trastuzumab and docetaxel for HER2-positive metastatic breast cancer. Clin Cancer Res 19: 4911-4916]. The HER2-specific monoclonal antibody Pertuzumab blocks HER2 dimerization with EGFR or HER3 and is FDA-approved for use in combination with Trastuzumab and Docetaxel. Unfortunately, >30% of patients experience cardiotoxicity [Ogiso H, Ishitani R, Nureki O, Fukai S, Yamanaka M, et al. (2002) Crystal structure of the complex of human epidermal growth factor and receptor extracellular domains. Cell 110: 775-787] or other serious side effects including anaphylaxis/hypersensitivity reactions with this combination therapy. Thus, although targeted therapeutics are available for the treatment of HER2-positive breast cancer, many patients still die from metastatic disease due to primary or acquired resistance, and these therapies are still associated with adverse reactions.

HER2 overexpression in breast cancer is associated with poor prognosis, but the advent of HER2-targeted antibodies such as Trastuzumab (Herceptin) and Pertuzumab, and ER2/EGFR tyrosine kinase inhibitors such as Lapatinib, have revolutionized the treatment of HER2-positive breast cancer. Unfortunately, 66-88% of HER2-positive tumors exhibit primary resistance to Trastuzumab as a monotherapy [Baselga, J., Tripathy, D., Mendelsohn, J., Baughman, S., Benz, C. C., Dantis, L., Sklarin, N. T., Seidman, A. D., Hudis, C. A., Moore, J., Rosen, P. P., Twaddell, T., Henderson, I. C., and Norton, L. (1996) Phase II study of weekly intravenous recombinant humanized anti-p185HER2 monoclonal antibody in patients with HER2/neu-overexpressing metastatic breast cancer J Clin Oncol 14, 737-744; Vogel, C. L., Cobleigh, M. A., Tripathy, D., Gutheil, J. C., Harris, L. N., Fehrenbacher, L., Slamon, D. J., Murphy, M., Novotny, W. F., Burchmore, M., Shak, S., Stewart, S. J., and Press, M. (2002) Efficacy and safety of trastuzumab as a single agent in first-line treatment of HER2-overexpressing metastatic breast cancer J Clin Oncol 20, 719-726; Cobleigh, M. A., Vogel, C. L., Tripathy, D., Robert, N. J., Scholl, S., Fehrenbacher, L., Wolter, J. M., Paton, V., Shak, S., Lieberman, G., and Slamon, D. J. (1999) Multinational study of the efficacy and safety of humanized anti-HER2 monoclonal antibody in women who have HER2-overexpressing metastatic breast cancer that has progressed after chemotherapy for metastatic disease J Clin Oncol 17, 2639-2648]. Further, standard Trastuzumab-centered regimens include either a Taxane or an Anthracycline to provide acceptable anti-cancer efficacy, but 15% of patients acquire resistance to these combination therapies as well [Piccart-Gebhart, M. J., Procter, M., Leyland-Jones, B., Goldhirsch, A., Untch, M., Smith, I., Gianni, L., Baselga, J., Bell, R., Jackisch, C., Cameron, D., Dowsett, M., Barrios, C. H., Steger, G., Huang, C. S., Andersson, M., Inbar, M., Lichinitser, M., Lang, I., Nitz, U., Iwata, H., Thomssen, C., Lohrisch, C., Suter, T. M., Ruschoff, J., Suto, T., Greatorex, V., Ward, C., Straehle, C., McFadden, E., Dolci, M. S., and Gelber, R. D. (2005) Trastuzumab after adjuvant chemotherapy in HER2-positive breast cancer N Engl J Med 353, 1659-1672]. These regimens are associated with significant side effects including cardiotoxicity and anaphylaxis [McKeage, K., and Perry, C. M. (2002) Trastuzumab: a review of its use in the treatment of metastatic breast cancer overexpressing HER2 Drugs 62, 209-243]. Clearly, additional therapies are needed to reduce the toxicity of HER2-targeted therapies and to overcome drug resistance.

A large number of resistance mechanisms to Trastuzumab and Lapatinib have been described [Vu, T., and Claret, F. X. (2012) Trastuzumab: updated mechanisms of action and resistance in breast cancer Front Oncol 2, 62; Hutchinson, L. (2010) Targeted therapies: Activated PI3K/AKT confers resistance to trastuzumab but not lapatinib Nat Rev Clin Oncol 7, 424; Wang, Y. C., Morrison, G., Gillihan, R., Guo, J., Ward, R. M., Fu, X., Botero, M. F., Healy, N. A., Hilsenbeck, S. G., Phillips, G. L., Chamness, G. C., Rimawi, M. F., Osborne, C. K., and Schiff, R. (2011) Different mechanisms for resistance to trastuzumab versus lapatinib in HER2-positive breast cancers-role of estrogen receptor and HER2 reactivation Breast Cancer Res 13, R121; Gayle, S. S., Arnold, S. L., O'Regan, R. M., and Nahta, R. (2012) Pharmacologic inhibition of mTOR improves lapatinib sensitivity in HER2-overexpressing breast cancer cells with primary trastuzumab resistance Anticancer Agents Med Chem 12, 151-162]. Many of these mechanisms involve the ability of these three proteins to function in a partially redundant manner. For example, when Trastuzumab inactivates HER2, EGFR and HER3 can still heterodimerize and drive mitogenic and survival signaling [Narayan, M., Wilken, J. A., Harris, L. N., Baron, A. T., Kimbler, K. D., and Maihle, N. J. (2009) Trastuzumab-induced HER reprogramming in “resistant” breast carcinoma cells Cancer Res 69, 2191-2194]. Likewise, Pertuzumab blocks HER2 dimerization with EGFR or HER3, but does not preclude EGFR/HERS dimerization and signaling. Lapatinib blocks the kinase activity of both HER2 and EGFR. While HER3 has very little intrinsic tyrosine kinase activity [Kok A., Terwisscha van Scheltinga, A. G., Timmer-Bosscha, H., Lamberts, L. E., Bensch, F., de Vries, E. G., and Schroder, C. P. (2014) HER3, serious partner in crime: therapeutic approaches and potential biomarkers for effect of HER3-targeting Pharmacol Ther 143, 1-11; Gullick, W. J. (1996) The c-erbB3/HER3 receptor in human cancer Cancer Surv 27, 339-349], it can serve as a substrate for c-Met and activate PI3K-dependent signaling in the absence of EGFR and HER2 function [Engelman, J. A., Zejnullahu, K., Mitsudomi, T., Song, Y., Hyland, C., Park, J. O., Lindeman, N., Gale, C. M., Zhao, X., Christensen, J., Kosaka, T., Holmes, A. J., Rogers, A. M., Cappuzzo, F., Mok, T., Lee, C., Johnson, B. E., Cantley, L. C., and Janne, P. A. (2007) MET amplification leads to gefitinib resistance in lung cancer by activating ERBB3 signaling Science 316, 1039-1043; Minuti, G., Cappuzzo, F., Duchnowska, R., Jassem, J., Fabi, A., O'Brien, T., Mendoza, A. D., Landi, L., Biernat, W., Czartoryska-Arlukowicz, B., Jankowski, T., Zuziak, D., Zok, J., Szostakiewicz, B., Foszczynska-Kloda, M., Tempinska-Szalach, A., Rossi, E., and Varella-Garcia, M. (2012) Increased MET and HGF gene copy numbers are associated with trastuzumab failure in HER2-positive metastatic breast cancer Br J Cancer 107, 793-799; Chen, C. T., Kim, H., Liska, D., Gao, S., Christensen, J. G., and Weiser, M. R. (2012) MET activation mediates resistance to lapatinib inhibition of HER2-amplified gastric cancer cells Mol Cancer Ther 11, 660-669]. Therefore, an improved agent for the treatment of HER2-dependent breast cancer would inactivate EGFR, HER2, and HER3 in parallel, be effective in the treatment of cancer as a single agent, and be mechanistically complementary with the HER2-targeted monoclonal antibodies and tyrosine kinase inhibitors.

Examination of the extracellular domains of EGFR, HER2, and HER3 [Garrett T P, McKern N M, Lou M, Elleman T C, Adams T E, et al. (2003) The crystal structure of a truncated ErbB2 ectodomain reveals an active conformation, poised to interact with other ErbB receptors. Mol Cell 11: 495-505; Cho H S, Mason K, Ramyar K X, Stanley A M, Gabelli S B, et al. (2003) Structure of the extracellular region of HER2 alone and in complex with the Herceptin Fab. Nature 421: 756-760; Cho H S, Leahy D J (2002) Structure of the extracellular region of HER3 reveals an interdomain tether. Science 297: 1330-1333; Field L, Khim Y H (1972) Organic disulfides and related substances. 33. Sodium 4-(2-acetamidoethyldithio)butanesulfinate and related compounds as antiradiation drugs. J Med Chem 15: 312-315] reveals a complicated pattern of structural repeats that are held in place by disulfide bonds. Agents capable of disrupting disulfide bonds may preferentially destabilize the structures of HER2, EGFR, and HER3 and inhibit their oncogenic functions. Optimal disulfide bond disrupting agents (DDAs) would target extracellular disulfide bonds, be charged at physiological pH to minimize entry into cells in order to reduce off-target effects, and would employ chemistry that does not affect nucleic acids. DDAs meeting these criteria are expected to be toxic to cancer cells that depend on HER2 for proliferation and survival, but to be well tolerated by normal tissues. Herein we describe the identification of a class of molecules that fulfill these criteria.

### SUMMARY OF THE INVENTION

In one aspect, the invention provides a compound of Formula I, or salt, solvate, hydrate or prodrug thereof:

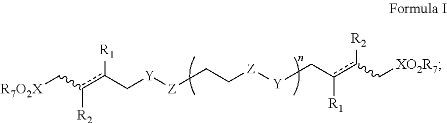

wherein, each X is independently S or Se;

each Y is independently S or Se;

each Z is independently S or Se;

each R₁ is independently selected from H, NH₂, N₃, OH, oxo, NH—R₃,

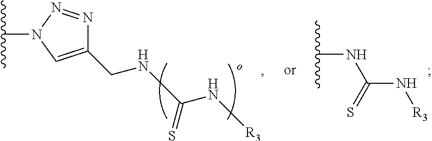

each R₂ is independently selected from H, NH₂, N₃, OH, oxo, NH—R₃,

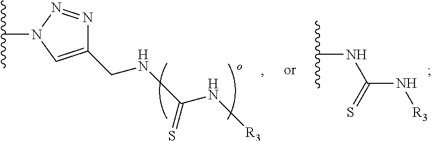

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol] denotes a carbon-carbon single bond or double bond;

wherein if every X, Y, and Z is simultaneously S, then at least one of R₁ and R₂ is NH₂, N₃, OH, oxo, NH—R₃,

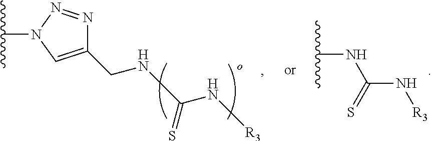

In another aspect, the compound of Formula I is

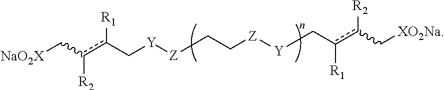

In another aspect, the invention provides a compound of Formula I, or salt, solvate, hydrate or prodrug thereof:

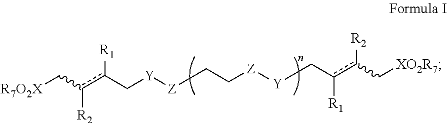

wherein, each X is independently S or Se;

each Y is independently S, SO₂, or Se;

each Z is independently S, SO₂, or Se;

each R₁ is independently selected from H, NH₂, N₃, OH, oxo, OAc, NH—R₃,

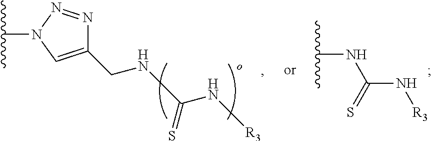

each R₂ is independently selected from H, NH₂, N₃, OH, oxo, OAc, NH—R₃,

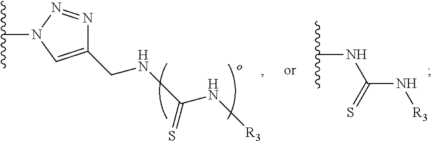

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol] denotes a carbon-carbon single bond or double bond;

wherein if every X, Y, and Z is simultaneously S, then at least one of R₁ and R₂ is NH₂, N₃, OH, oxo, OAc, NH—R₃,

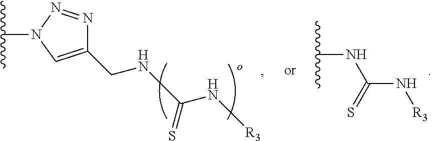

In another aspect, the compound of Formula I is

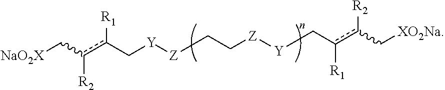

In another aspect, the invention provides a compound of Formula II, or salt, solvate, hydrate or prodrug thereof:

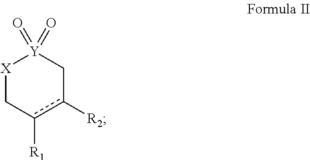

wherein, X is S or Se;

Y is S or Se;

R₁ is selected from H, NH₂, N₃, or OH;

R₂ is selected from H, NH₂, N₃, or OH;

or R₁, R₂, and the carbon atoms to which they are attached form an optionally substituted cycloalkyl moiety; and

[symbol] denotes a carbon-carbon single bond or double bond;

wherein if X and Y are both S, then at least one of R₁ and R₂ is NH₂, N₃, or OH.

In another aspect, the invention provides a compound of Formula II, or salt, solvate, hydrate or prodrug thereof:

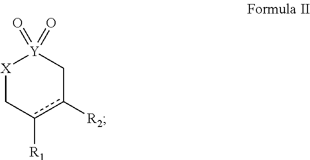

wherein, X is S or Se;

Y is S or Se;

R₁ is selected from H, NH₂, N₃, OAc, alkyl, or OH;

R₂ is selected from H, NH₂, N₃, OAc, alkyl, or OH;

or R₁, R₂, and the carbon atoms to which they are attached form an optionally substituted cycloalkyl moiety or optionally substituted aryl moiety; and

[symbol] denotes a carbon-carbon single bond or double bond;

wherein if X and Y are both S, then at least one of R₁ and R₂ is NH₂, N₃, OAc, alkyl, or OH.

In one aspect, the invention provides a method of treating a subject suffering from or susceptible to a cell proliferative disorder. The method includes administering to a subject in need thereof a therapeutically effective amount of a compound of Formula I, or salt, solvate, hydrate or prodrug thereof:

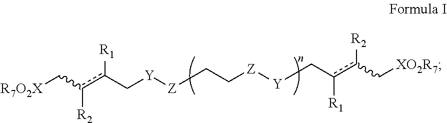

wherein, each X is independently S or Se;

each Y is independently S or Se;

each Z is independently S or Se;

each R₁ is independently selected from H, NH₂, N₃, OH, oxo, NH—R₃,

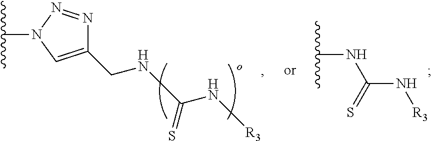

each R₂ is independently selected from H, NH₂, N₃, OH, oxo, NH—R₃,

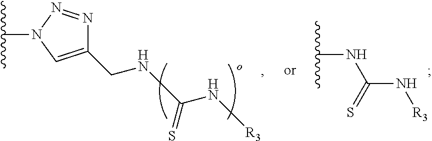

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol] denotes a carbon-carbon single bond or double bond. In another aspect, the compound of Formula I is

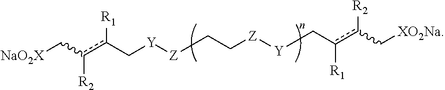

In another aspect, the cell proliferative disorder is cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In one aspect, the invention provides a method of treating a subject suffering from or susceptible to a cell proliferative disorder. The method includes administering to a subject in need thereof a therapeutically effective amount of a compound of Formula I, or salt, solvate, hydrate or prodrug thereof:

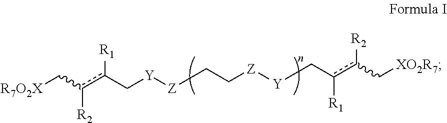

wherein, each X is independently S or Se;

each Y is independently S, SO₂, or Se;

each Z is independently S, SO₂, or Se;

each R₁ is independently selected from H, NH₂, N₃, OH, oxo, OAc, NH—R₃,

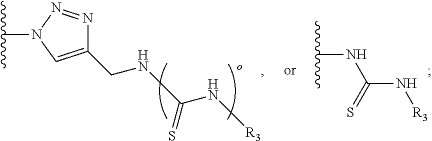

each R₂ is independently selected from H, NH₂, N₃, OH, oxo, OAc, NH—R₃,

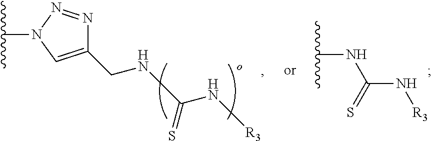

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol] denotes a carbon-carbon single bond or double bond. In another aspect, the compound of Formula I is

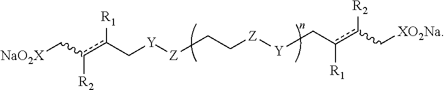

In another aspect, the cell proliferative disorder is cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a method of treating a subject suffering from or susceptible to a cell proliferative disorder. The method includes administering to a subject in need thereof a therapeutically effective amount of a compound of Formula II, or salt, solvate, hydrate or prodrug thereof:

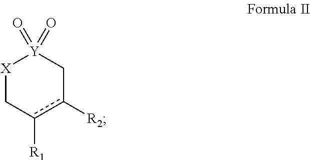

wherein, X is S or Se;

Y is S or Se;

R₁ is selected from H, NH₂, N₃, or OH;

R₂ is selected from H, NH₂, N₃, or OH;

or R₁, R₂, and the carbon atoms to which they are attached form an optionally substituted cycloalkyl moiety; and

[symbol] denotes a carbon-carbon single bond or double bond. In another aspect, the cell proliferative disorder is cancer. In a further aspect, the cancer is HER2 mediated. In a further aspect, the cancer is breast cancer. In a further aspect, the breast cancer is HER2-positive breast cancer. In another aspect, the breast cancer is driven by HER2, HER3, and/or EGFR.

In another aspect, the invention provides a method of treating a subject suffering from or susceptible to a cell proliferative disorder. The method includes administering to a subject in need thereof a therapeutically effective amount of a compound of Formula II, or salt, solvate, hydrate or prodrug thereof:

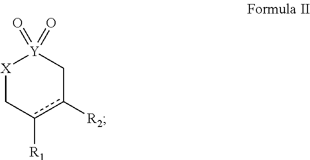

wherein, X is S or Se;

Y is S or Se;

R₁ is selected from H, NH₂, N₃, OAc, alkyl, or OH;

R₂ is selected from H, NH₂, N₃, OAc, alkyl, or OH;

or R₁, R₂, and the carbon atoms to which they are attached form an optionally substituted cycloalkyl moiety or optionally substituted aryl moiety; and

[symbol] denotes a carbon-carbon single bond or double bond. In another aspect, the cell proliferative disorder is cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a method of inhibiting cell proliferation. The method includes administering to the cell a therapeutically effective amount of a compound of Formula I, or salt, solvate, hydrate or prodrug thereof:

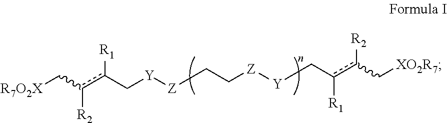

wherein, each X is independently S or Se;

each Y is independently S or Se;

each Z is independently S or Se;

each R₁ is independently selected from H, NH₂, N₃, OH, oxo, NH—R₃,

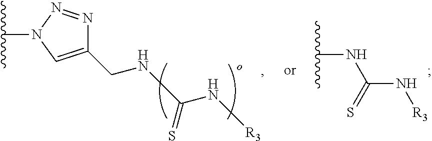

each R₂ is independently selected from H, NH₂, N₃, OH, oxo, NH—R₃,

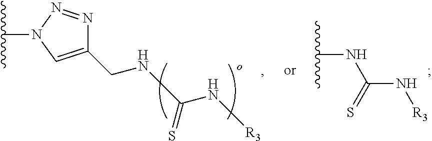

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol]

denotes a carbon-carbon single bond or double bond. In another aspect, the compound of Formula I is

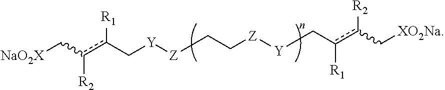

In another aspect, the cell proliferative disorder is cancer. In a further aspect, the cancer cell is HER2 mediated. In a further aspect, the cancer cell is breast cancer. In a further aspect, the breast cancer cell is HER2-positive breast cancer cell. In another aspect, the breast cancer cell is modulated by HER2, HER3, and/or EGFR.

In another aspect, the invention provides a method of inhibiting cell proliferation. The method includes administering to the cell a therapeutically effective amount of a compound of Formula I, or salt, solvate, hydrate or prodrug thereof:

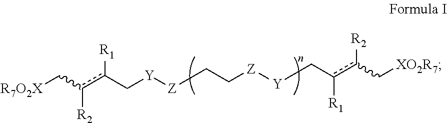

wherein, each X is independently S or Se;

each Y is independently S, SO₂, or Se;

each Z is independently S, SO₂, or Se;

each R₁ is independently selected from H, NH₂, N₃, OH, oxo, OAc, NH—R₃,

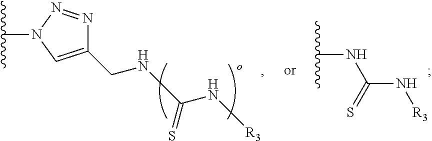

each R₂ is independently selected from H, NH₂, N₃, OH, oxo, OAc, NH—R₃,

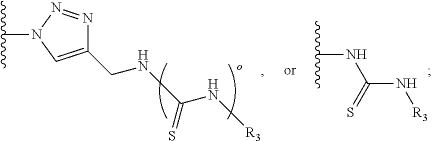

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol] denotes a carbon-carbon single bond or double bond. In another aspect, the compound of Formula I is

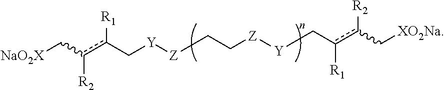

In another aspect, the cell proliferative disorder is cancer. In a further aspect, the cancer cell is HER2 mediated. In a further aspect, the cancer cell is breast cancer. In a further aspect, the breast cancer cell is HER2-positive breast cancer cell. In another aspect, the breast cancer cell is modulated by HER2, HER3, and/or EGFR.

In another aspect, the invention provides a method of inhibiting cell proliferation. The method includes administering to the cell a therapeutically effective amount of a compound of Formula II, or salt, solvate, hydrate or prodrug thereof:

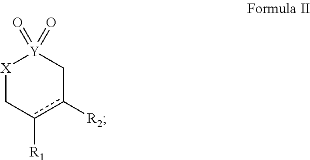

wherein, X is S or Se;

Y is S or Se;

R₁ is selected from H, NH₂, N₃, or OH;

R₂ is selected from H, NH₂, N₃, or OH;

or R₁, R₂, and the carbon atoms to which they are attached form an optionally substituted cycloalkyl moiety; and

[symbol] denotes a carbon-carbon single bond or double bond. In another aspect, the cell proliferative disorder is cancer. In a further aspect, the cancer cell is HER2 mediated. In a further aspect, the cancer cell is breast cancer. In a further aspect, the breast cancer cell is HER2-positive breast cancer cell. In another aspect, the breast cancer cell is modulated by HER2, HER3, and/or EGFR.

In another aspect, the invention provides a method of inhibiting cell proliferation. The method includes administering to the cell a therapeutically effective amount of a compound of Formula II, or salt, solvate, hydrate or prodrug thereof:

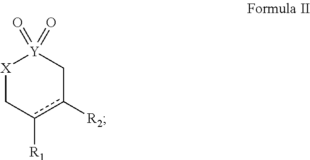

wherein, X is S or Se;

Y is S or Se;

R₁ is selected from H, NH₂, N₃, OAc, alkyl, or OH;

R₂ is selected from H, NH₂, N₃, OAc, alkyl, or OH;

or R₁, R₂, and the carbon atoms to which they are attached form an optionally substituted cycloalkyl moiety or an optionally substituted aryl moiety; and

[symbol] denotes a carbon-carbon single bond or double bond. In another aspect, the cell proliferative disorder is cancer. In a further aspect, the cancer cell is HER2 mediated. In a further aspect, the cancer cell is breast cancer. In a further aspect, the breast cancer cell is HER2-positive breast cancer cell. In another aspect, the breast cancer cell is modulated by HER2, HER3, and/or EGFR.

In another aspect, the invention provides a method of inhibiting cancer cell metastasis. The method includes administering to a subject in need thereof a therapeutically effective amount of a compound of Formula I, or salt, solvate, hydrate or prodrug thereof:

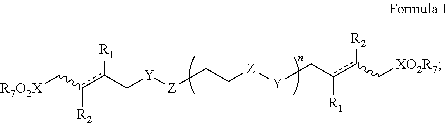

wherein, each X is independently S or Se;

each Y is independently S or Se;

each Z is independently S or Se;

each R₁ is independently selected from H, NH₂, N₃, OH, oxo, NH—R₃,

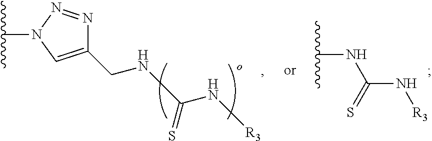

each R₂ is independently selected from H, NH₂, N₃, OH, oxo, NH—R₃,

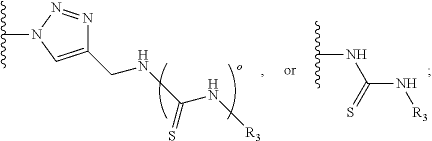

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol]

denotes a carbon-carbon single bond or double bond. In another aspect, the compound of Formula I is

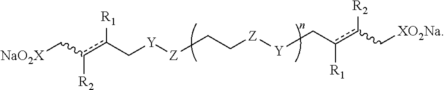

In another aspect, the cell proliferative disorder is cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a method of inhibiting cancer cell metastasis. The method includes administering to a subject in need thereof a therapeutically effective amount of a compound of Formula I, or salt, solvate, hydrate or prodrug thereof:

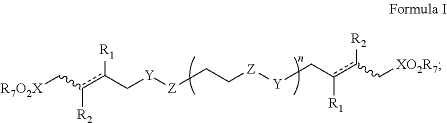

wherein, each X is independently S or Se;

each Y is independently S, SO₂, or Se;

each Z is independently S, SO₂, or Se;

each R₁ is independently selected from H, NH₂, N₃, OH, oxo, OAc, NH—R₃,

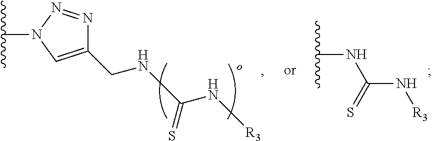

each R₂ is independently selected from H, NH₂, N₃, OH, oxo, OAc, NH—R₃,

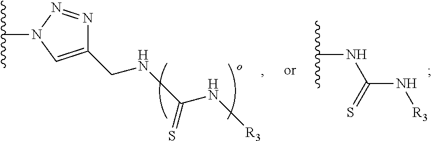

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol]

denotes a carbon-carbon single bond or double bond. In another aspect, the compound of Formula I is

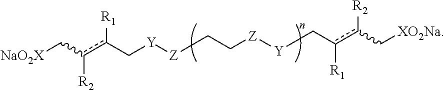

In another aspect, the cell proliferative disorder is cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a method of inhibiting cancer cell metastasis. The method includes administering to a subject in need thereof a therapeutically effective amount of a compound of Formula II, or salt, solvate, hydrate or prodrug thereof:

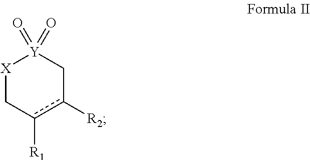

wherein, X is S or Se;

Y is S or Se;

R₁ is selected from H, NH₂, N₃, or OH;

R₂ is selected from H, NH₂, N₃, or OH;

or R₁, R₂, and the carbon atoms to which they are attached form an optionally substituted cycloalkyl moiety; and

[symbol] denotes a carbon-carbon single bond or double bond. In another aspect, the cell proliferative disorder is cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a method of inhibiting cancer cell metastasis. The method includes administering to a subject in need thereof a therapeutically effective amount of a compound of Formula II, or salt, solvate, hydrate or prodrug thereof:

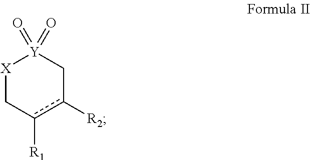

wherein, X is S or Se;

Y is S or Se;

R₁ is selected from H, NH₂, N₃, OAc, alkyl, or OH;

R₂ is selected from H, NH₂, N₃, OAc, alkyl, or OH;

or R₁, R₂, and the carbon atoms to which they are attached form an optionally substituted cycloalkyl moiety or an optionally substituted aryl moiety; and

[symbol] denotes a carbon-carbon single bond or double bond. In another aspect, the cell proliferative disorder is cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a kit for treating a cell proliferative disorder in a subject. The kit includes a compound of Formula I, or salt, solvate, hydrate or prodrug thereof:

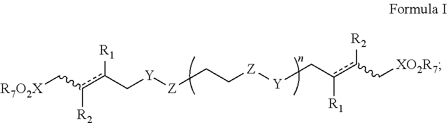

wherein, each X is independently S or Se;

each Y is independently S or Se;

each Z is independently S or Se;

each R₁ is independently selected from H, NH₂, N₃, OH, oxo, NH—R₃,

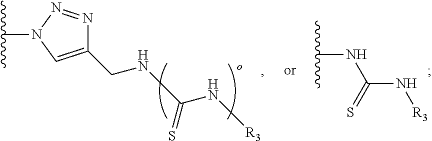

each R₂ is independently selected from H, NH₂, N₃, OH, oxo, NH—R₃,

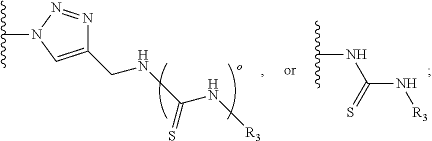

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol]

denotes a carbon-carbon single bond or double bond; and instructions for use. In another aspect, the compound of Formula I is

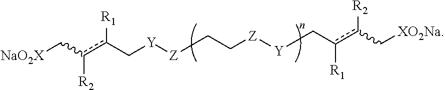

In certain embodiments, the invention provides kits for inhibiting cell proliferation, assessing the efficacy of an anti-cell proliferative treatment in a subject, monitoring the progress of a subject being treated with a cell proliferation inhibitor, selecting a subject with a cell proliferative disorder for treatment with cell proliferation inhibitor, and/or treating a subject suffering from or susceptible to cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a kit for treating a cell proliferative disorder in a subject. The kit includes a compound of Formula I, or salt, solvate, hydrate or prodrug thereof:

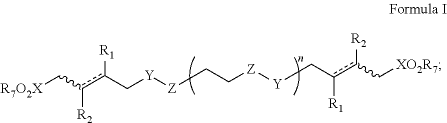

wherein, each X is independently S or Se;

each Y is independently S, SO₂, or Se;

each Z is independently S, SO₂, or Se;

each R₁ is independently selected from H, NH₂, N₃, OH, oxo, OAc, NH—R₃,

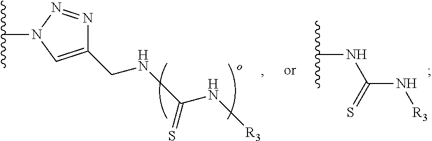

each R₂ is independently selected from H, NH₂, N₃, OH, oxo, OAc, NH—R₃,

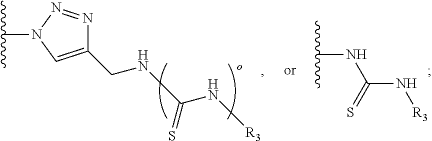

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol]

denotes a carbon-carbon single bond or double bond; and instructions for use. In another aspect, the compound of Formula I is

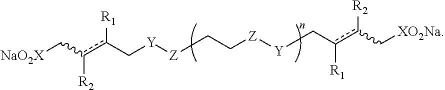

In certain embodiments, the invention provides kits for inhibiting cell proliferation, assessing the efficacy of an anti-cell proliferative treatment in a subject, monitoring the progress of a subject being treated with a cell proliferation inhibitor, selecting a subject with a cell proliferative disorder for treatment with cell proliferation inhibitor, and/or treating a subject suffering from or susceptible to cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a kit for treating a cell proliferative disorder in a subject. The kit includes a compound of Formula II, or salt, solvate, hydrate or prodrug thereof:

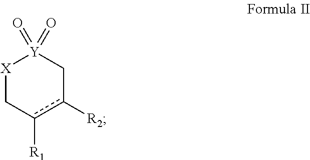

wherein, X is S or Se;

Y is S or Se;

R₁ is selected from H, NH₂, N₃, or OH;

R₂ is selected from H, NH₂, N₃, or OH;

or R₁, R₂, and the carbon atoms to which they are attached form an optionally substituted cycloalkyl moiety; and

[symbol] denotes a carbon-carbon single bond or double bond; and instructions for use. In certain embodiments, the invention provides kits for inhibiting cell proliferation, assessing the efficacy of an anti-cell proliferative treatment in a subject, monitoring the progress of a subject being treated with a cell proliferation inhibitor, selecting a subject with a cell proliferative disorder for treatment with cell proliferation inhibitor, and/or treating a subject suffering from or susceptible to cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a kit for treating a cell proliferative disorder in a subject. The kit includes a compound of Formula II, or salt, solvate, hydrate or prodrug thereof:

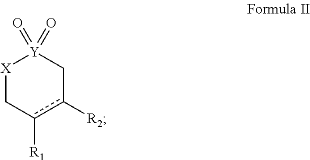

wherein, X is S or Se;

Y is S or Se;

R₁ is selected from H, NH₂, N₃, OAc, alkyl, or OH;

R₂ is selected from H, NH₂, N₃, OAc, alkyl, or OH;

or R₁, R₂, and the carbon atoms to which they are attached form an optionally substituted cycloalkyl moiety or an optionally substituted aryl moiety; and

[symbol] denotes a carbon-carbon single bond or double bond; and instructions for use. In certain embodiments, the invention provides kits for inhibiting cell proliferation, assessing the efficacy of an anti-cell proliferative treatment in a subject, monitoring the progress of a subject being treated with a cell proliferation inhibitor, selecting a subject with a cell proliferative disorder for treatment with cell proliferation inhibitor, and/or treating a subject suffering from or susceptible to cancer. In a further aspect, the cancer is HER2 mediated. In a further aspect, the cancer is breast cancer. In a further aspect, the breast cancer is HER2-positive breast cancer. In another aspect, the breast cancer is modulated by HER2, HER3, and/or EGFR.

In another aspect, the invention provides a method of inhibiting EGFR, HER2, and/or HER3. The method includes administering a therapeutically effective amount of a compound of Formula I, or salt, solvate, hydrate or prodrug thereof:

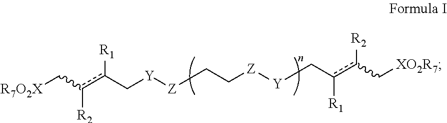

wherein, each X is independently S or Se;

each Y is independently S or Se;

each Z is independently S or Se;

each R₁ is independently selected from H, NH₂, N₃, OH, oxo, NH—R₃,

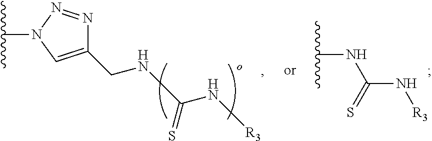

each R₂ is independently selected from H, NH₂, N₃, OH, oxo, NH—R₃,

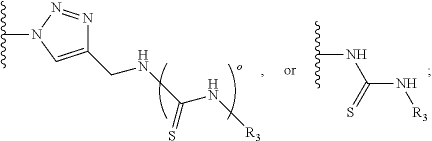

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol] denotes a carbon-carbon single bond or double bond. In another aspect, the compound of Formula I is

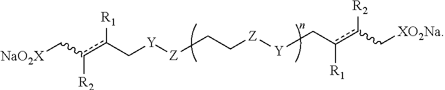

In another aspect, the compound inhibits or is capable of inhibiting at least two of EGFR, HER2, and HER3. In another aspect, the compound inhibits or is capable of inhibiting all three of EGFR, HER2, and HER3.

In another aspect, the invention provides a method of inhibiting EGFR, HER2, and/or HER3. The method includes administering a therapeutically effective amount of a compound of Formula I, or salt, solvate, hydrate or prodrug thereof:

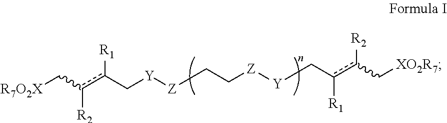

wherein, each X is independently S or Se;

each Y is independently S, SO₂, or Se;

each Z is independently S, SO₂, or Se;

each R₁ is independently selected from H, NH₂, N₃, OH, oxo, OAc, NH—R₃,

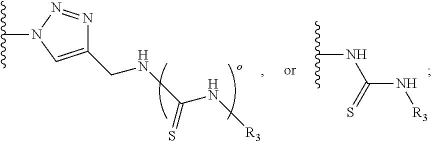

each R₂ is independently selected from H, NH₂, N₃, OH, oxo, OAc, NH—R₃,

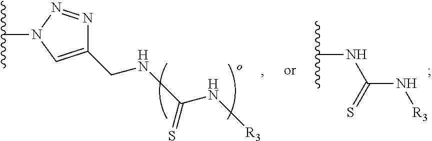

each R₃ is independently selected from biotin, fluorescein, AlexaFluor® dyes, BODIPY®, Cascade Blue®, coumarins, Oregon Green®, Pacific Blue™, Pacific Green™, Pacific Orange™, Rhodamine Green™, Rhodamine Red™, or Texas Red®;

or adjacent R₁, R₂ moieties, and the carbon atoms to which they are attached form an optionally substituted cycloalkyl moiety;

each R₇ is independently H, Na, K, optionally substituted alkyl, optionally substituted aryl, or optionally substituted arylalkyl;

each n is independently 0 or 1;

each o is independently 0 or 1; and

[symbol] denotes a carbon-carbon single bond or double bond. In another aspect, the compound of Formula I is

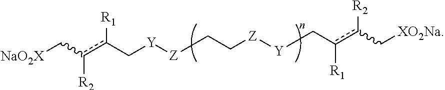

In another aspect, the compound inhibits or is capable of inhibiting at least two of EGFR, HER2, and HER3. In another aspect, the compound inhibits or is capable of inhibiting all three of EGFR, HER2, and HER3.

In another aspect, the invention provides a method of inhibiting EGFR, HER2, and/or HER3. The method includes administering a therapeutically effective amount of a compound of Formula II, or salt, solvate, hydrate or prodrug thereof:

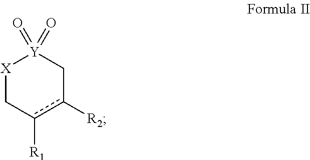

wherein, X is S or Se;

Y is S or Se;

R₁ is selected from H, NH₂, N₃, or OH;

R₂ is selected from H, NH₂, N₃, or OH;

or R₁, R₂, and the carbon atoms to which they are attached form an optionally substituted cycloalkyl moiety; and

[symbol]

denotes a carbon-carbon single bond or double bond. In another aspect, the compound inhibits or is capable of inhibiting at least two of EGFR, HER2, and HER3. In another aspect, the compound inhibits or is capable of inhibiting all three of EGFR, HER2, and HER3.

In another aspect, the invention provides a method of inhibiting EGFR, HER2, and/or HER3. The method includes administering a therapeutically effective amount of a compound of Formula II, or salt, solvate, hydrate or prodrug thereof:

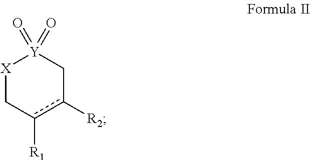

wherein, X is S or Se;

Y is S or Se;

R₁ is selected from H, NH₂, N₃, OAc, alkyl, or OH;

R₂ is selected from H, NH₂, N₃, OAc, alkyl, or OH;

or R₁, R₂, and the carbon atoms to which they are attached form an optionally substituted cycloalkyl moiety or an optionally substituted aryl moiety; and

[symbol]

denotes a carbon-carbon single bond or double bond. In another aspect, the compound inhibits or is capable of inhibiting at least two of EGFR, HER2, and HER3. In another aspect, the compound inhibits or is capable of inhibiting all three of EGFR, HER2, and HER3.

In another aspect, the invention provides a compound that is:
sodium (2R,3R)-2,3-diacetoxy-4-((2-(((2R,3R)-2,3-diacetoxy-4-sulfinatobutyl)disulfanyl)ethyl)disulfanyl)butane-1-sulfinate;
sodium (2S,3S)-2,3-diacetoxy-4-((2-(((2S,3S)-2,3-diacetoxy-4-sulfinatobutyl)disulfanyl)ethyl)disulfanyl)butane-1-sulfinate;
sodium (2S,3R)-2,3-diacetoxy-4-((2-(((2R,3S)-2,3-diacetoxy-4-sulfinatobutyl)disulfanyl)ethyl)disulfanyl)butane-1-sulfinate;
sodium (2R,3S)-2,3-diacetoxy-4-((2-(((2S,3R)-2,3-diacetoxy-4-sulfinatobutyl)disulfanyl)ethyl)disulfanyl)butane-1-sulfinate;
sodium 4-(2-(4-sulfinatobutylsulfonylthio)ethylthiosulfonyl)butane-1-sulfinate;
sodium 4-(2-(4-sulfinatobutylthiosulfonyl)ethylsulfonylthio)butane-1-sulfinate
sodium 4-(2-(4-sulfinatobutylsulfonylsulfonyl)ethylsulfonylsulfonyl)butane-1-sulfinate;
sodium 4,4′-diselanediyldibutane-1-seleninate;
sodium 5,10-dithia-6,9-diselenatetradecane-1,14-disulfinate;
sodium 6,9-dithia-5,10-diselenatetradecane-1,14-diseleninate;
sodium 4,4′-(ethane-1,2-diylbis(diselanediyl))dibutane-1-seleninate;
sodium (2Z,2′Z)-4,4′-disulfanediyldibut-2-ene-1-sulfinate;
sodium (2E,2′E)-5,5′-disulfanediyldipent-2-ene-1-sulfinate;
sodium (2R,2′R,3R,3′R)-4,4′-disulfanediylbis(2,3-dihydroxybutane-1-sulfinate);
sodium (2S,2′S,3S,3′S)-4,4′-disulfanediylbis(2,3-dihydroxybutane-1-sulfinate);
sodium (2R,2′R,3R,3′R)-4,4′-disulfanediylbis(2,3-diaminobutane-1-sulfinate);
sodium (2S,2′S,3S,3′S)-4,4′-disulfanediylbis(2,3-diaminobutane-1-sulfinate);
sodium (2R,2′R,3R,3′R)-4,4′-disulfanediylbis(2,3-diazidobutane-1-sulfinate);
sodium (2S,2′S,3S,3′S)-4,4′-disulfanediylbis(2,3-diazidobutane-1-sulfinate);
sodium 4,4′-disulfanediylbis(2,3-dioxobutane-1-sulfinate);
sodium (3R,3′R)-4,4′-disulfanediylbis(3-aminobutane-1-sulfinate);
sodium (3S,3′S)-4,4′-disulfanediylbis(3-aminobutane-1-sulfinate);
sodium (3R,3′R)-4,4′-disulfanediylbis(3-azidobutane-1-sulfinate);
sodium (3S,3′S)-4,4′-disulfanediylbis(3-azidobutane-1-sulfinate);
sodium (2R,2′R)-4,4′-disulfanediylbis(2-aminobutane-1-sulfinate);
sodium (2S,2′S)-4,4′-disulfanediylbis(2-aminobutane-1-sulfinate);
sodium (2R,2′R)-4,4′-disulfanediylbis(2-azidobutane-1-sulfinate);
sodium (2S,2′S)-4,4′-disulfanediylbis(2-azidobutane-1-sulfinate);
sodium (1R,1′R,2R,2′R,3R,3′R,4S,4′S)-3,3′-disulfanediylbis(methylene)bis(bicyclo[2.2.1]heptane-3,2-diyl)dimethanesulfinate;
sodium (1R,1′R,2S,2′S,3S,3′S,4S,4′S)-3,3′-disulfanediylbis(methylene)bis(bicyclo[2.2.1]heptane-3,2-diyl)dimethanesulfinate;
sodium (2R,2′R,3R,3′R)-4,4′-(ethane-1,2-diylbis(disulfanediyl))bis(2,3-dihydroxybutane-1-sulfinate);
sodium (2S,2′S,3S,3′S)-4,4′-(ethane-1,2-diylbis(disulfanediyl))bis(2,3-dihydroxybutane-1-sulfinate);
sodium (2R,2′R,3R,3′R)-4,4′-(ethane-1,2-diylbis(disulfanediyl))bis(2,3-diaminobutane-1-sulfinate);
(2S,2′S,3S,3′S)-4,4′-(ethane-1,2-diylbis(disulfanediyl))bis(2,3-diaminobutane-1-sulfinate);
(2R,2′R,3R,3′R)-4,4′-(ethane-1,2-diylbis(disulfanediyl))bis(2,3-diazidobutane-1-sulfinate);
sodium (2S,2′S,3S,3′S)-4,4′-(ethane-1,2-diylbis(disulfanediyl))bis(2,3-diazidobutane-1-sulfinate);
sodium 4,4′-(ethane-1,2-diylbis(disulfanediyl))bis(2,3-dioxobutane-1-sulfinate);
sodium (3R,3′R)-4,4′-(ethane-1,2-diylbis(disulfanediyl))bis(3-aminobutane-1-sulfinate);
sodium (3S,3′S)-4,4′-(ethane-1,2-diylbis(disulfanediyl))bis(3-aminobutane-1-sulfinate);
sodium (3R,3′R)-4,4′-(ethane-1,2-diylbis(disulfanediyl))bis(3-azidobutane-1-sulfinate);
sodium (3S,3′S)-4,4′-(ethane-1,2-diylbis(disulfanediyl))bis(3-azidobutane-1-sulfinate);
sodium (2R,2′R)-4,4′-(ethane-1,2-diylbis(disulfanediyl))bis(2-aminobutane-1-sulfinate);
sodium (2S,2′S)-4,4′-(ethane-1,2-diylbis(disulfanediyl))bis(2-aminobutane-1-sulfinate);
sodium (2R,2′R)-4,4′-(ethane-1,2-diylbis(disulfanediyl))bis(2-azidobutane-1-sulfinate);
sodium (2S,2′S)-4,4′-(ethane-1,2-diylbis(disulfanediyl))bis(2-azidobutane-1-sulfinate);
sodium (1R,1′R,2R,2′R,3R,3′R,4S,4′S)-3,3′-(ethane-1,2-diylbis(disulfanediyl))bis(methylene)bis(bicyclo[2.2.1]heptane-3,2-diyl)dimethanesulfinate;
sodium (1R,1′R,2S,2′S,3S,3′S,4S,4′S)-3,3′-(ethane-1,2-diylbis(disulfanediyl))bis(methylene)bis(bicyclo[2.2.1]heptane-3,2-diyl)dimethanesulfinate;
1,2-diselenane-1,1-dioxide;
3,6-dihydro-1,2-dithiine-1,1-dioxide;
trans-1,2-dithiane-4,5-diol-1,1-dioxide;
trans-1,2-dithiane-4,5-diamino-1,1-dioxide;
trans-1,2-dithiane-4,5-diazido-1,1-dioxide;
cis-1,2-dithiane-4,5-diol-1,1-dioxide;
cis-1,2-dithiane-4,5-diamino-1,1-dioxide;
cis-1,2-dithiane-4,5-diazido-1,1-dioxide;
1,2-dithiane-4,5-dione-1,1-dioxide;
1,2-dithiane-(4R,5S-diacetoxy)-1,1-dioxide;
1,2-dithiane-(4S,5R-diacetoxy)-1,1-dioxide;
1,2-dithiane-(4R,5R-diacetoxy)-1,1-dioxide;
1,2-dithiane-(4S,5S-diacetoxy)-1,1-dioxide;
1,2-dithiane-(4R,5S-dihydroxy)-1,1-dioxide;
1,2-dithiane-(4S,5R-dihydroxy)-1,1-dioxide;
1,2-dithiane-(4R,5R-dihydroxy)-1,1-dioxide;
1,2-dithiane-(4S,5S-dihydroxy)-1,1-dioxide;
1,2-dithiane-4-amino-1,1-dioxide;
1,2-dithiane-4-azido-1,1-dioxide;
1,2-dithiane-5-amino-1,1-dioxide;
1,2-dithiane-5-azido-1,1-dioxide;

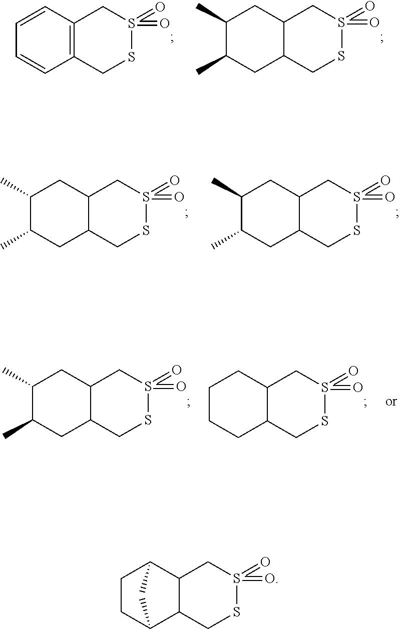

In another aspect, the invention provides a compound of Formula III, or salt, hydrate, solvate, or prodrug thereof:

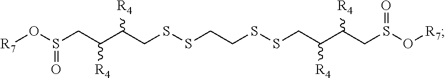

wherein, each R₄ is independently selected from

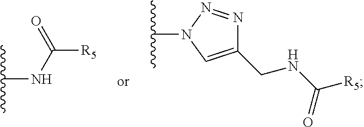

each R₅ is independently selected from the group consisting of:

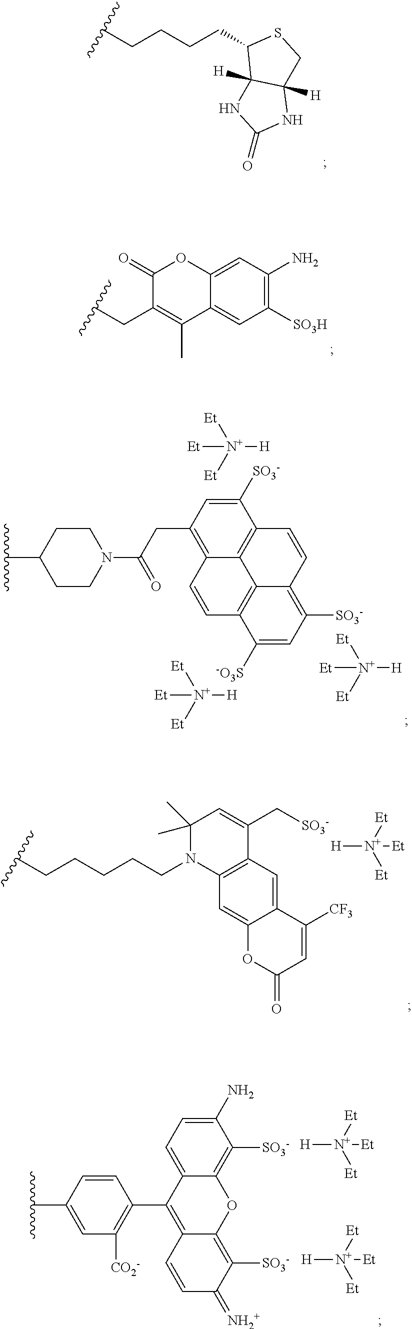

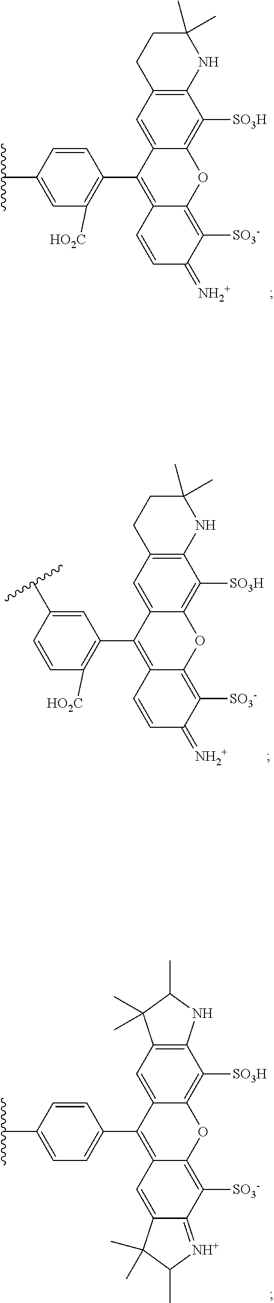

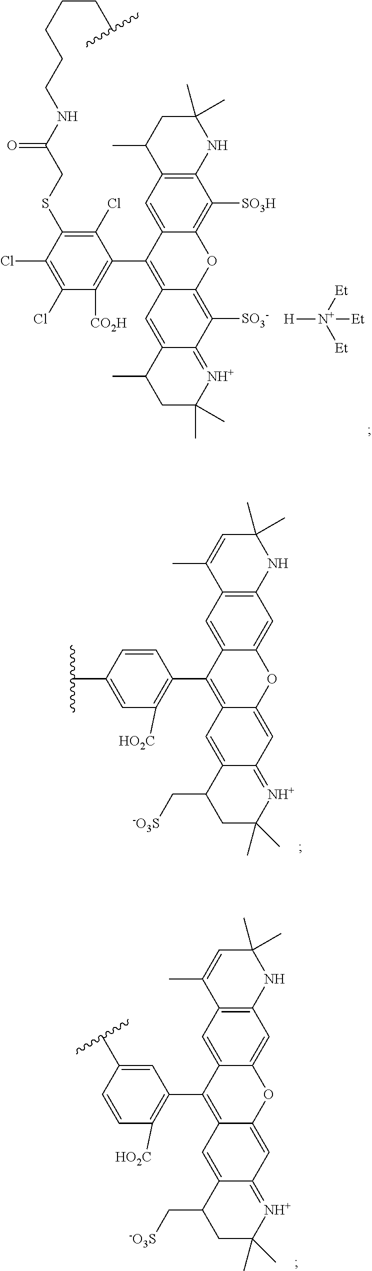

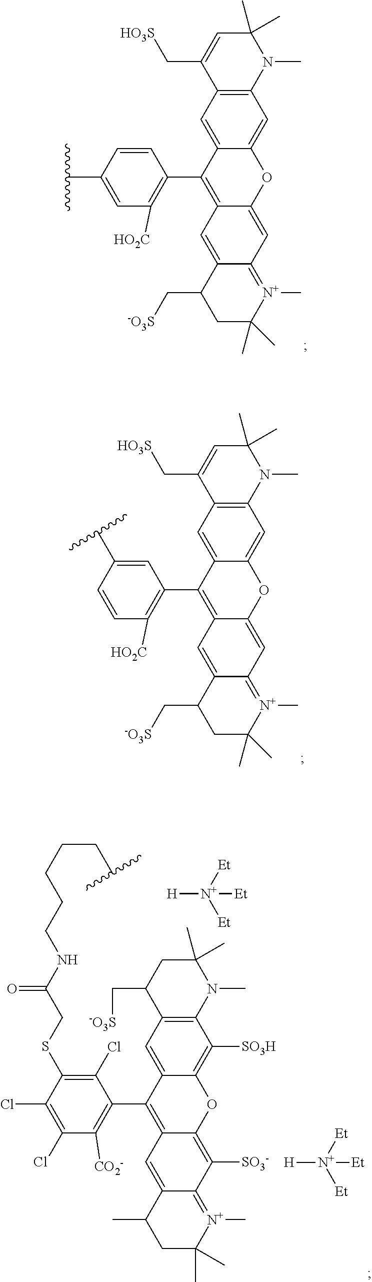

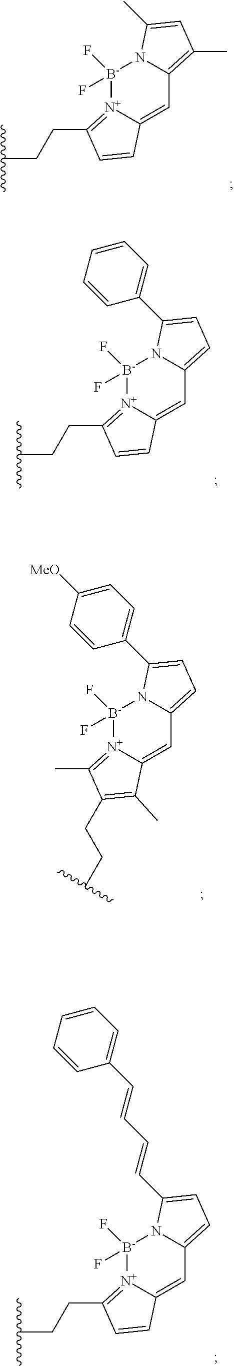 [SMILES: *CCC1=[N+]2C(=Cc3c(C)cc(C)n3[B-]2(F)F)C=C1 ; *CCC1=[N+]2C(=Cc3ccc(-c4ccccc4)n3[B-]2(F)F)C=C1 ; *CCC1=C(C)C2=Cc3ccc(-c4ccc(OC)cc4)n3[B-](F)(F)[N+]2=C1C ; *CCC1=[N+]2C(=Cc3ccc(/C=C/C=C/c4ccccc4)n3[B-]2(F)F)C=C1]

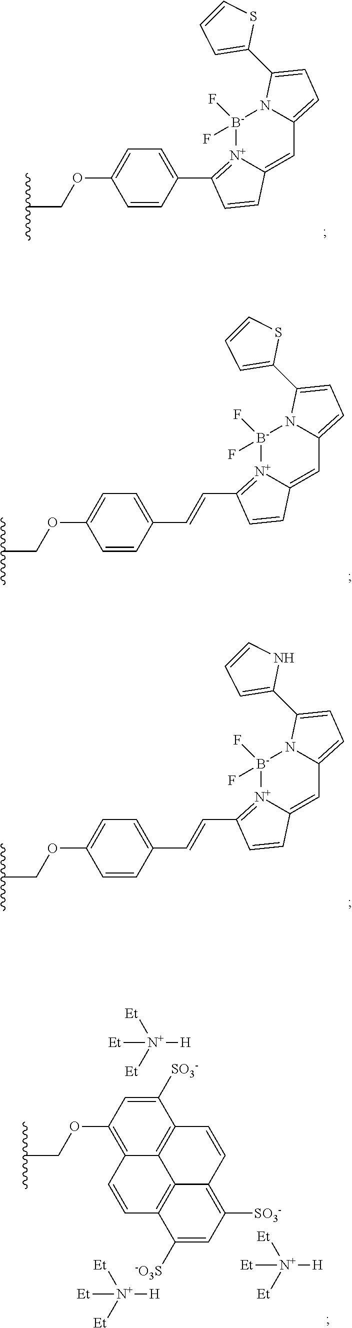

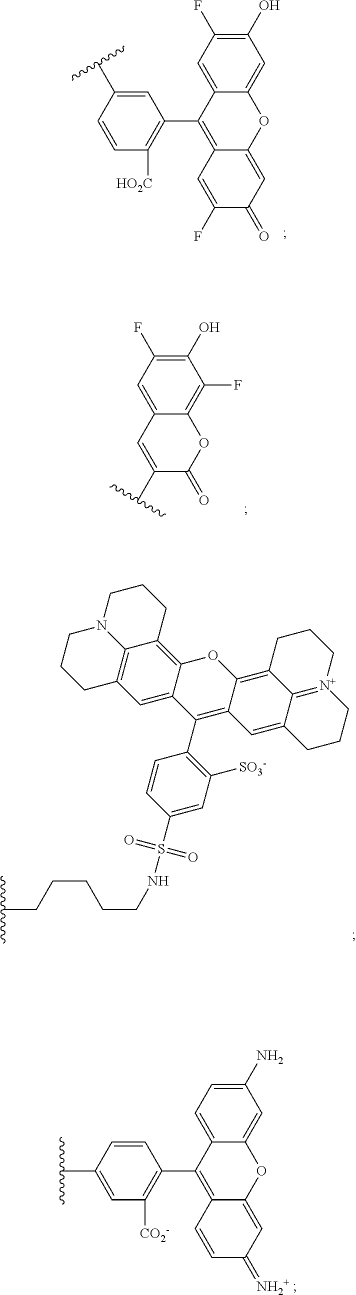

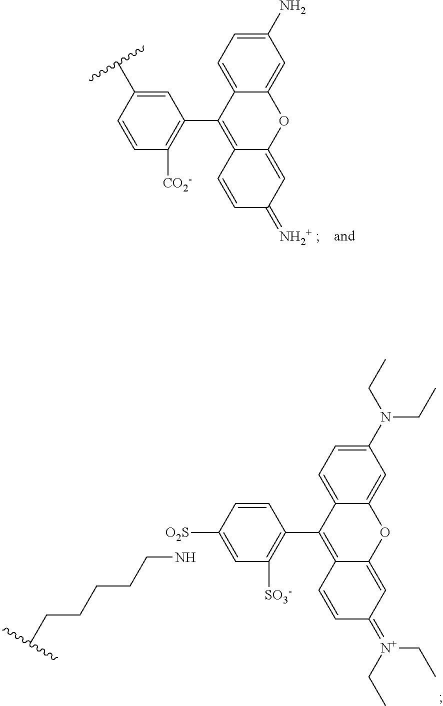

and

each R₇ is independently H, Na, K, optionally substituted alkyl, optionally substituted aryl, or optionally substituted arylalkyl. In another aspect, the compound of Formula III is represented by Formula V:

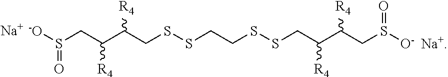

In another aspect, the invention provides a compound of Formula V, or salt, hydrate, solvate, or prodrug thereof:

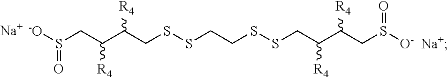

wherein the compound is selected from the group consisting of:

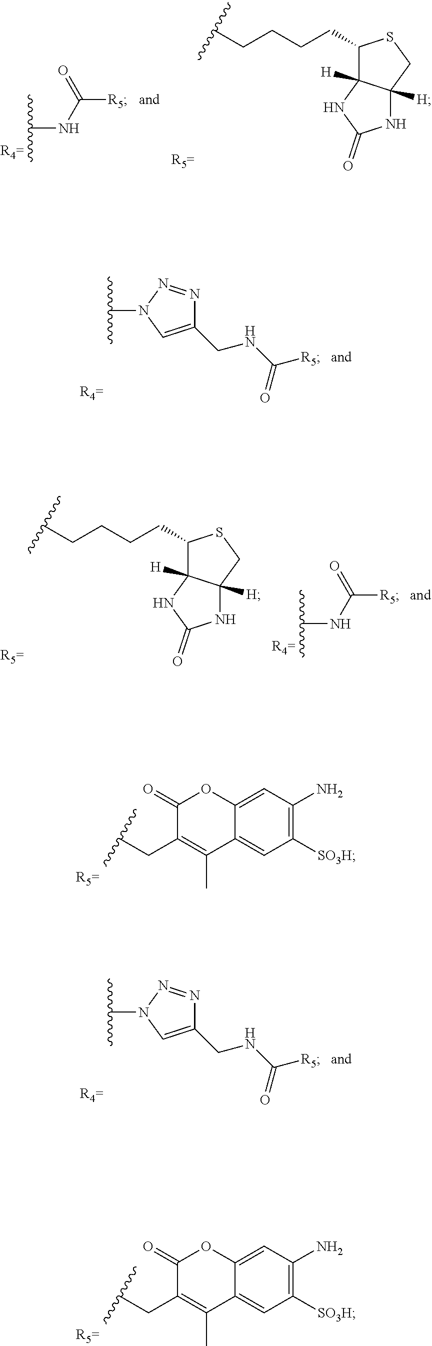

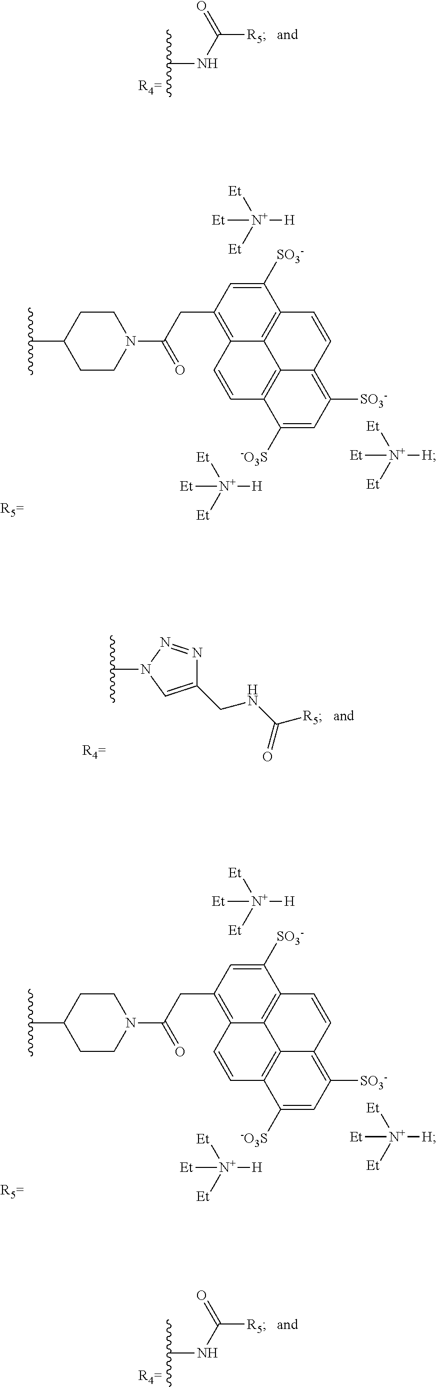

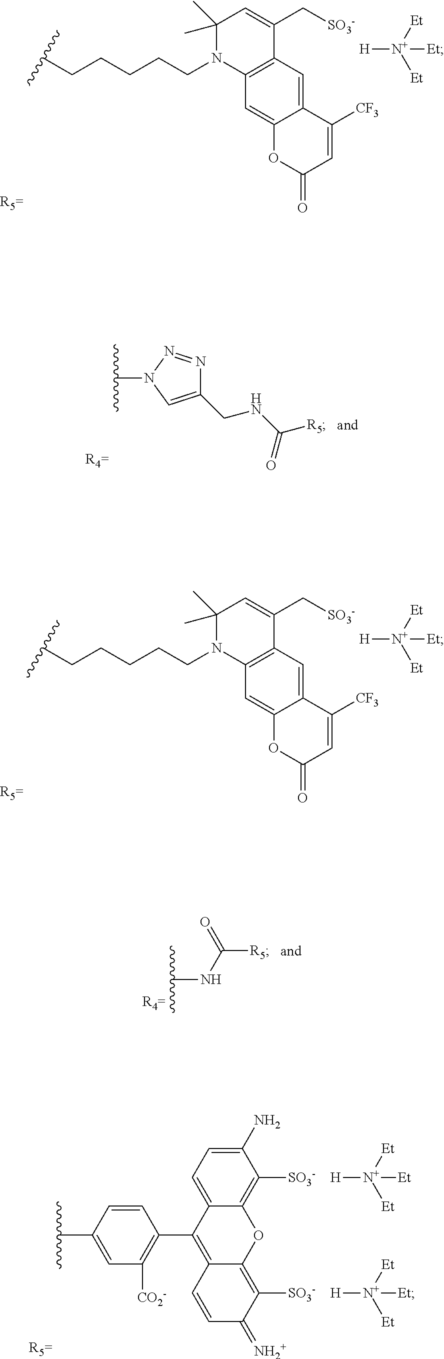

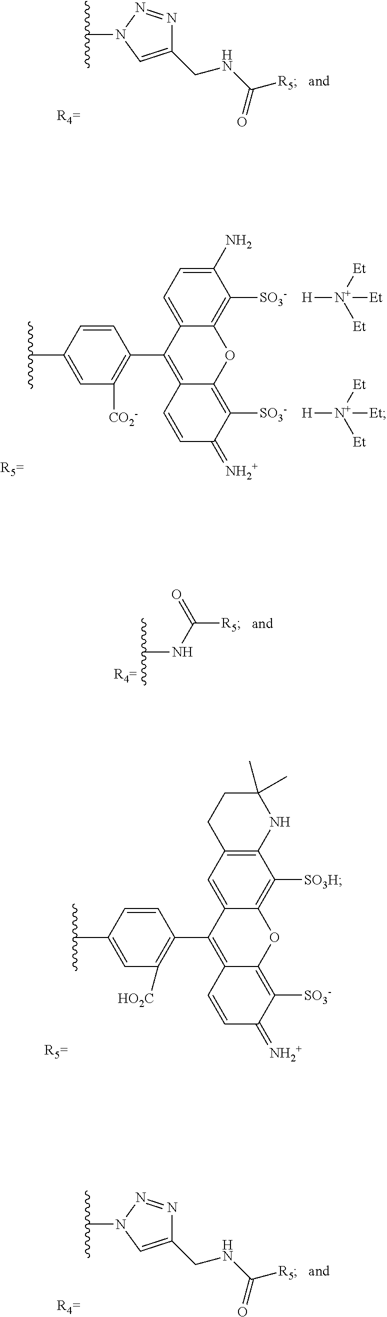

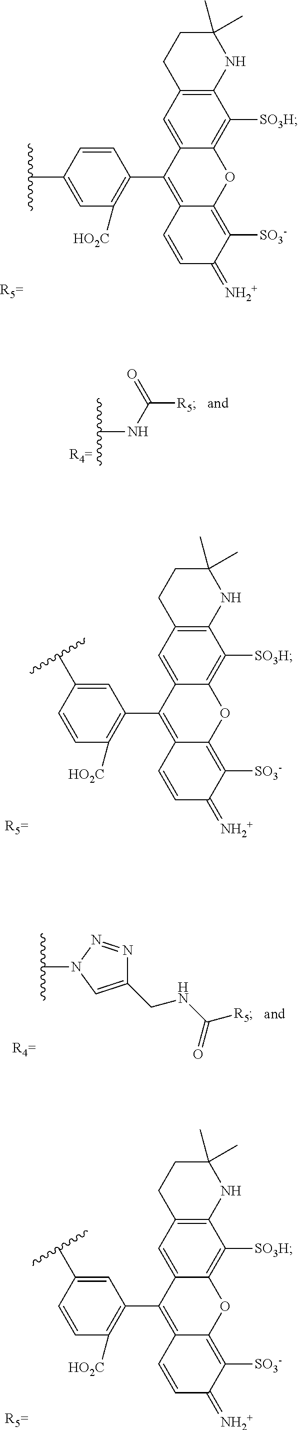

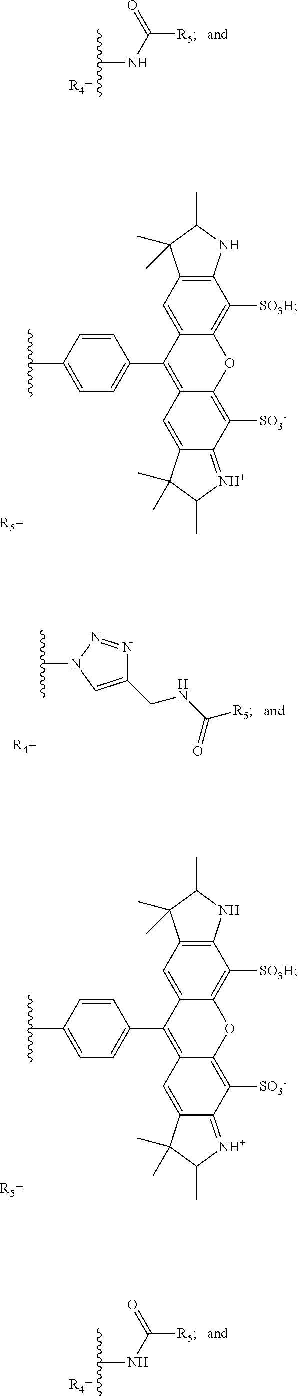

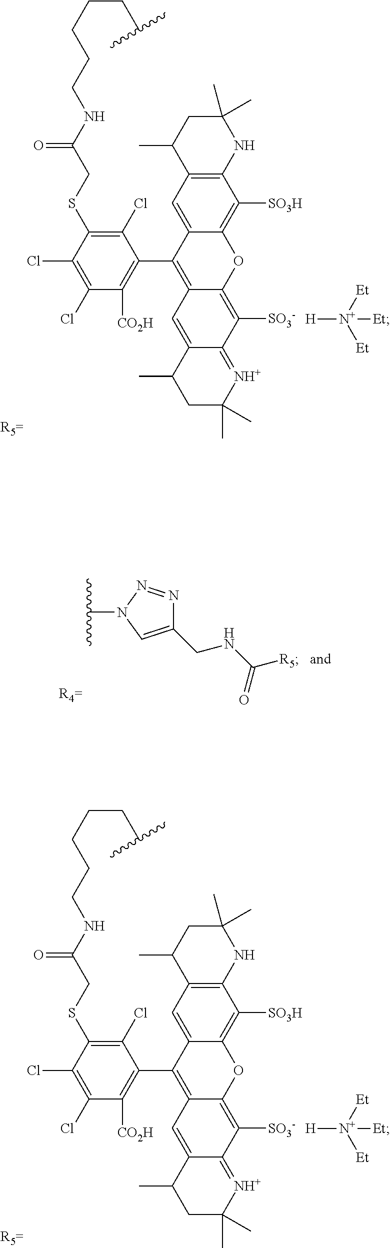

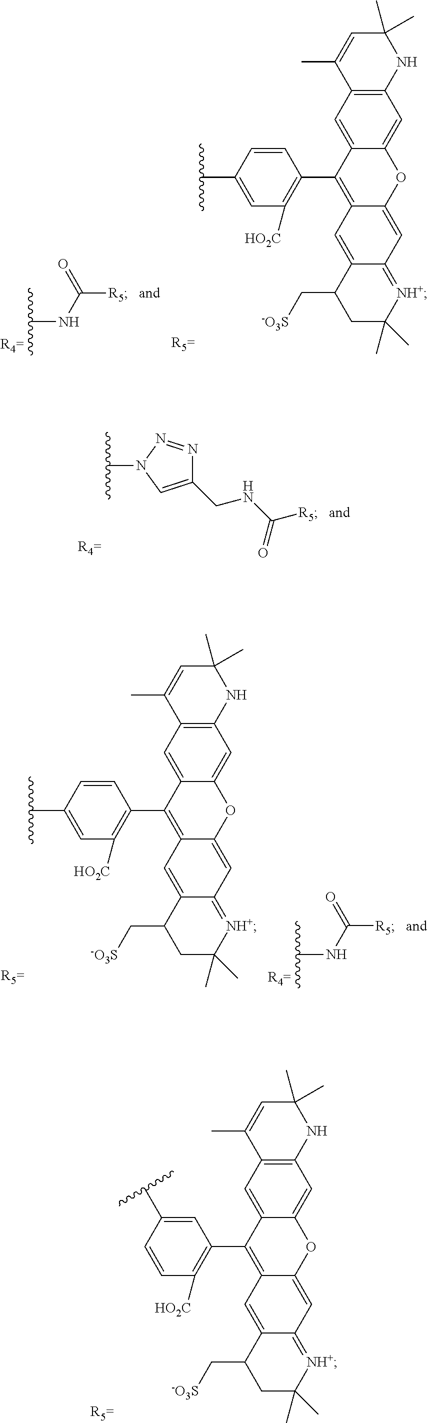

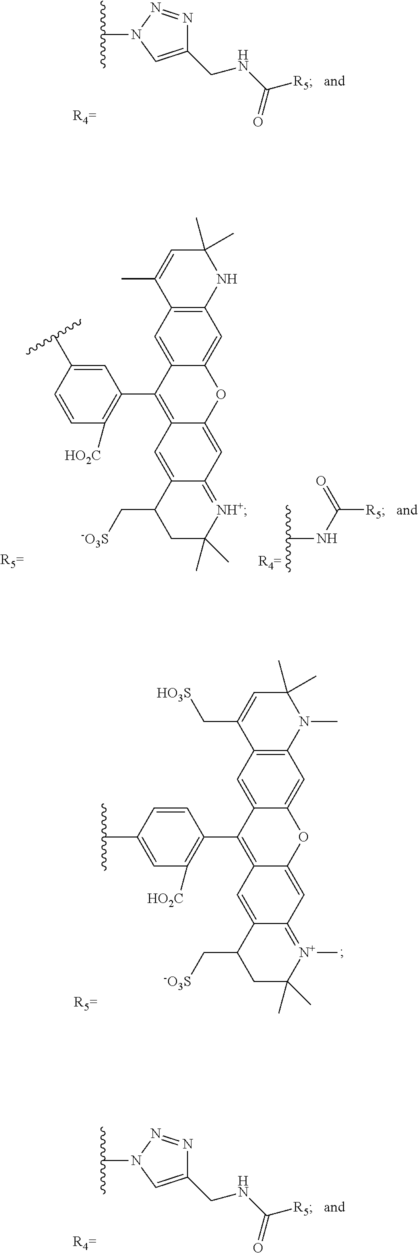

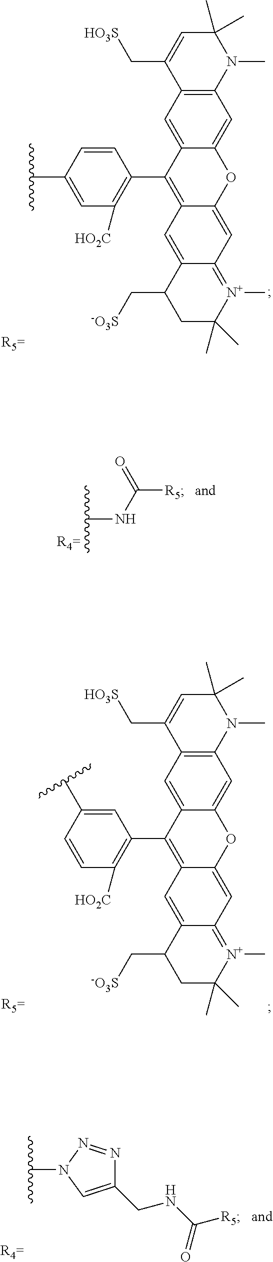

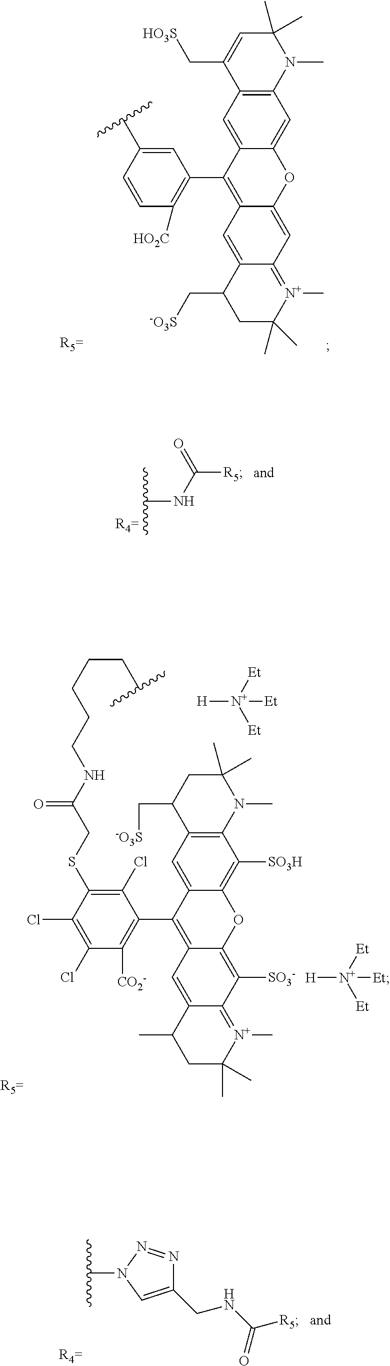

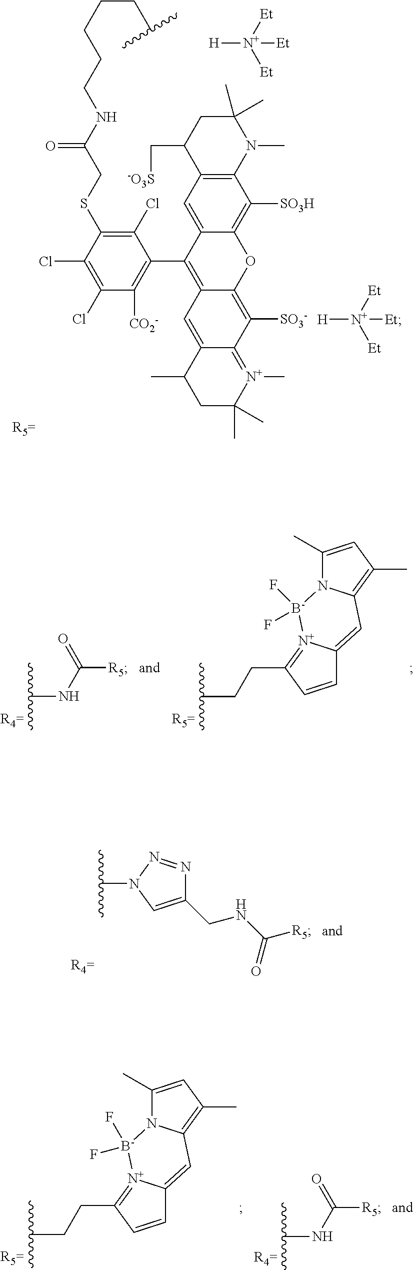

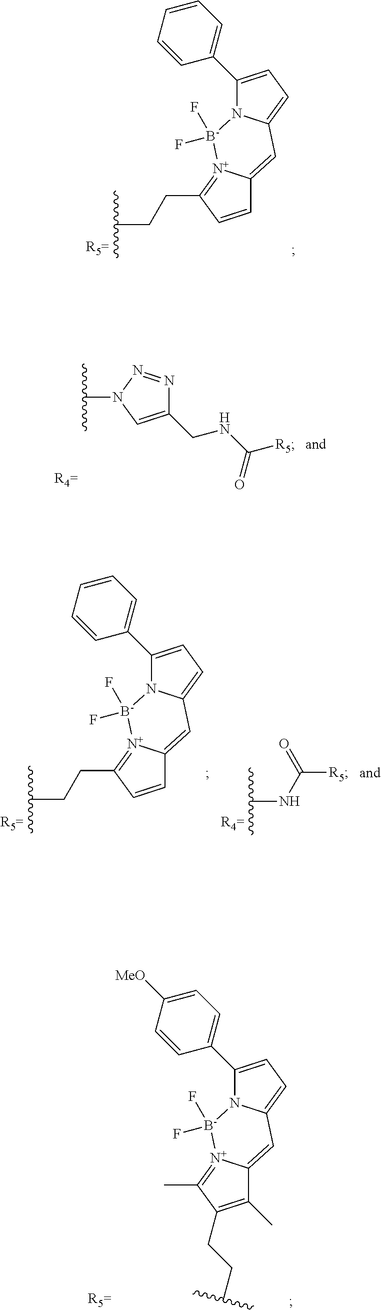

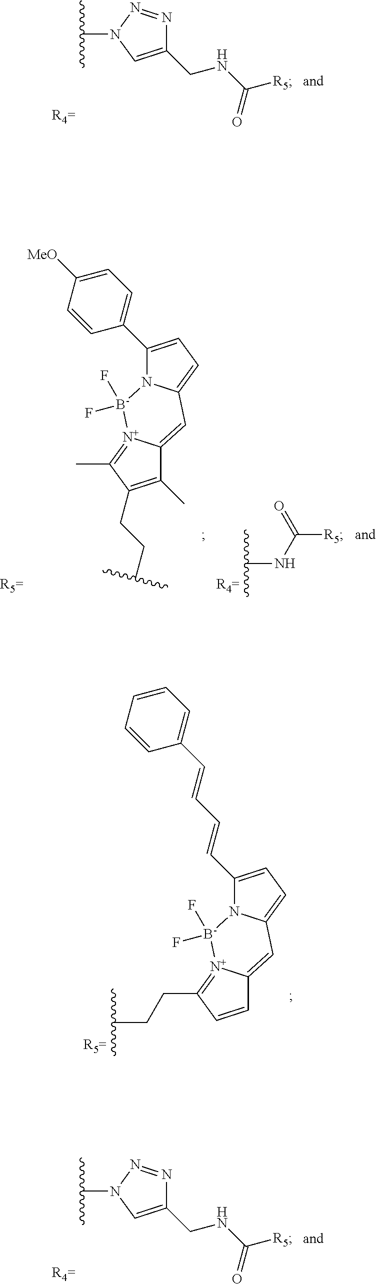

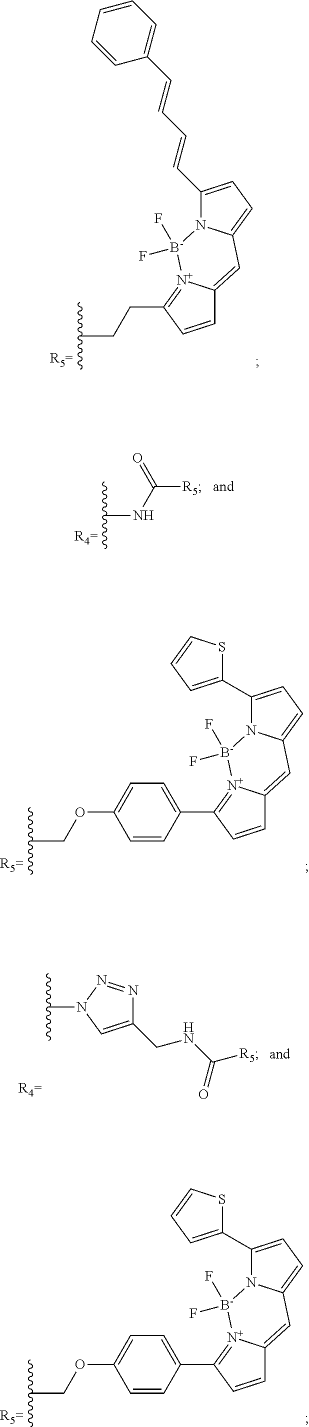

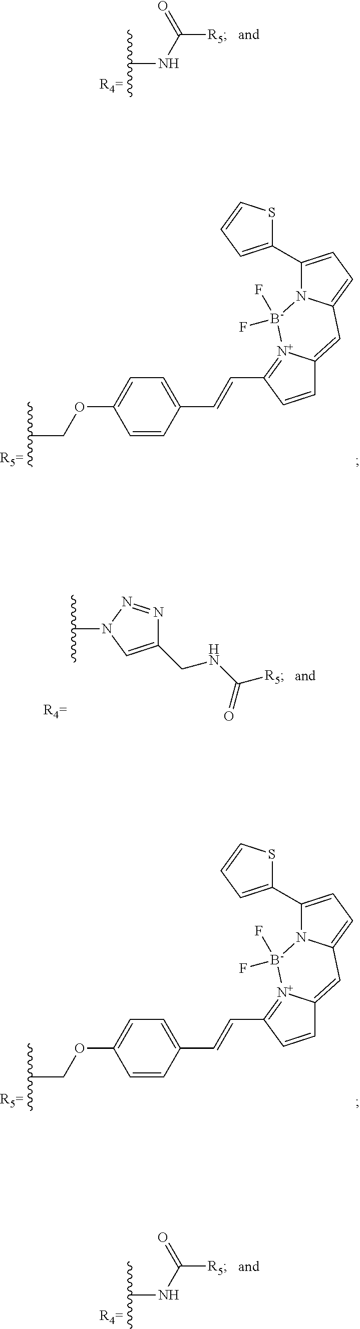

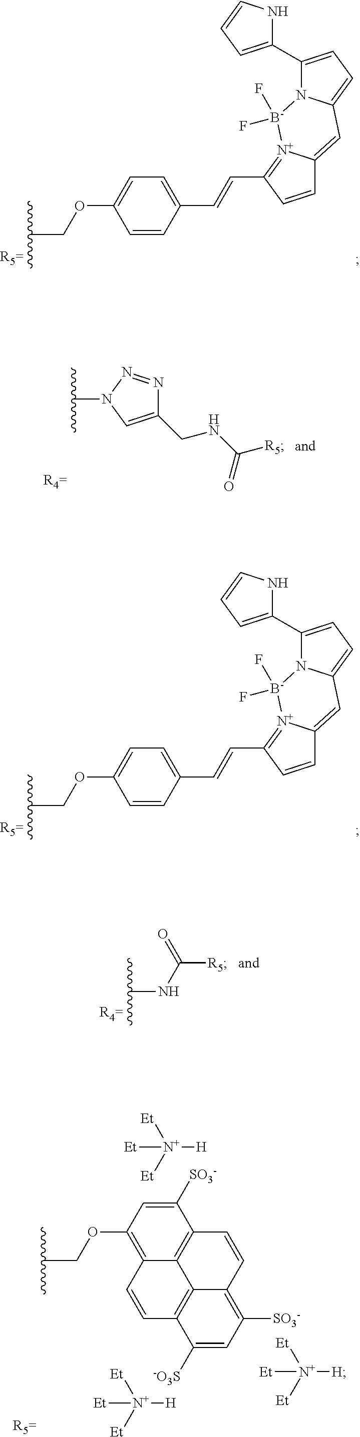

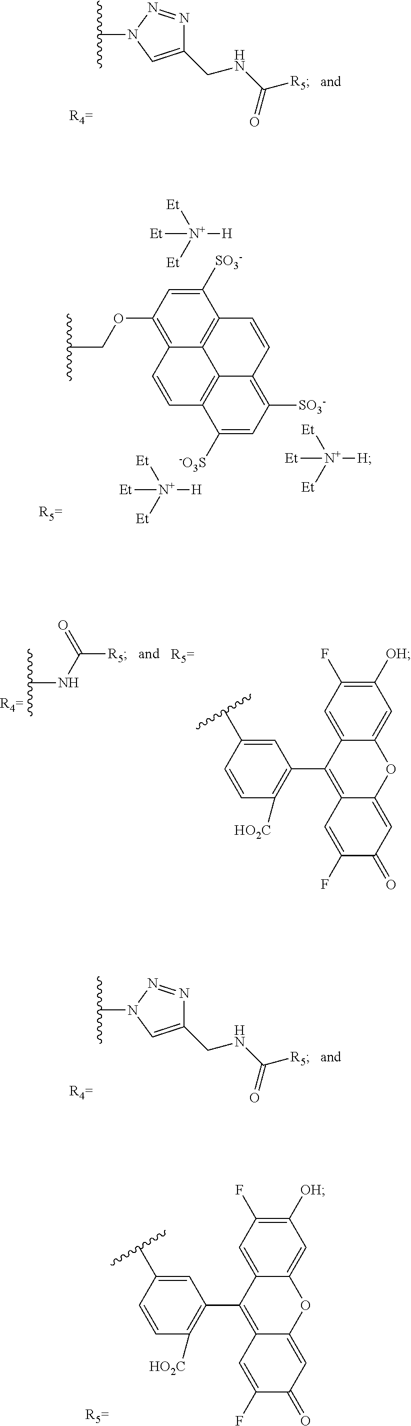

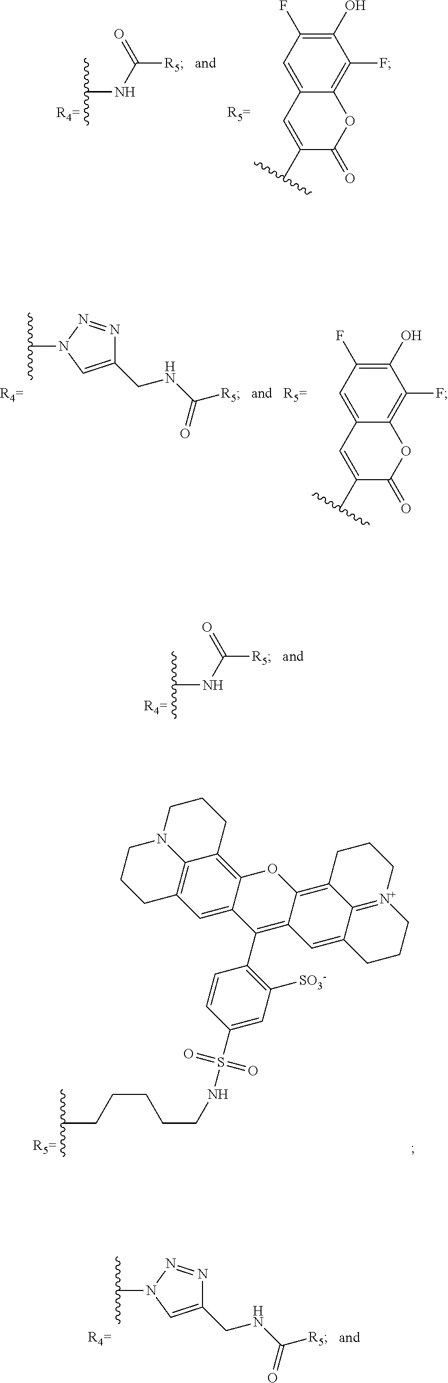

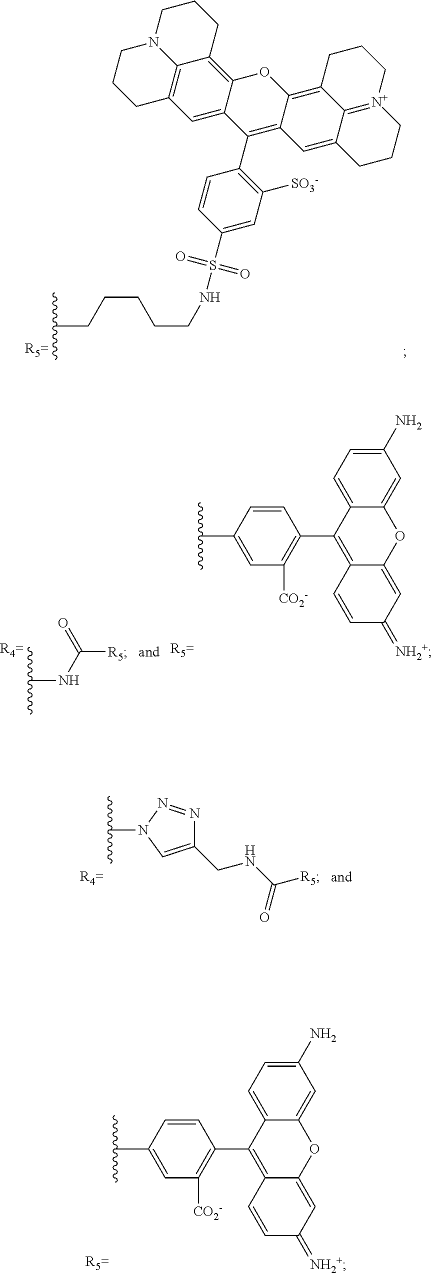

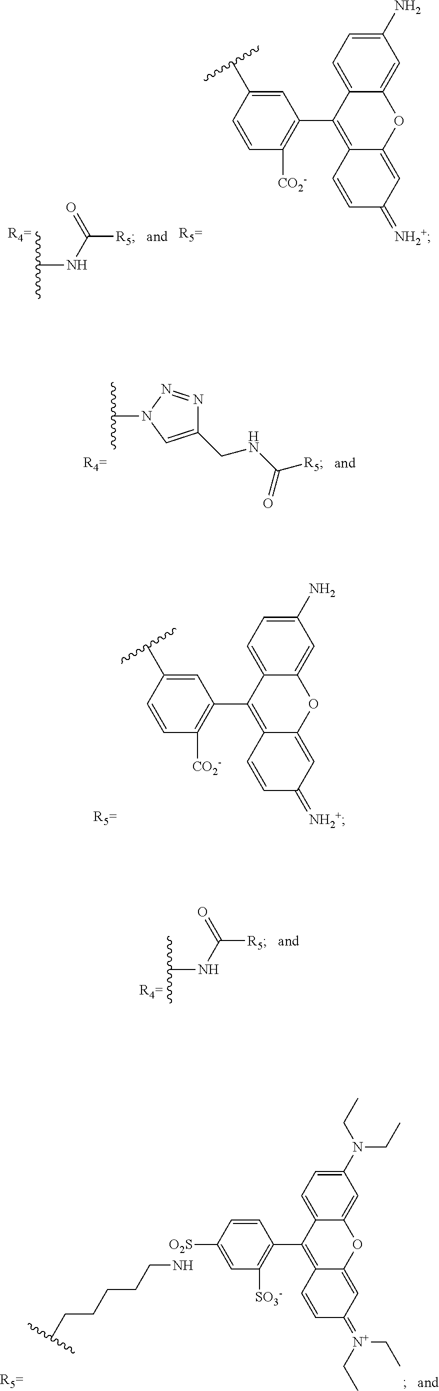

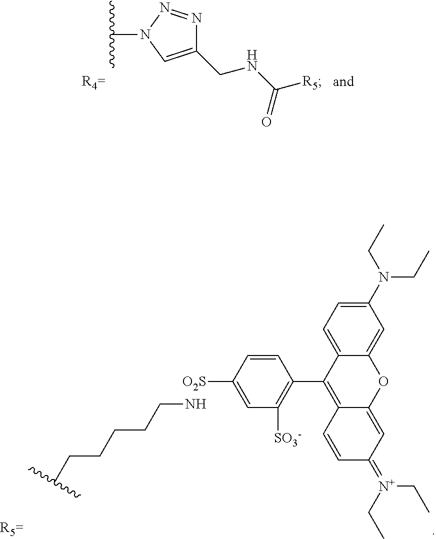

In another aspect, the invention provides a compound of Formula IV, or salt, hydrate, solvate, or prodrug thereof:

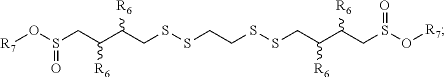

wherein, each R₆ is independently selected from

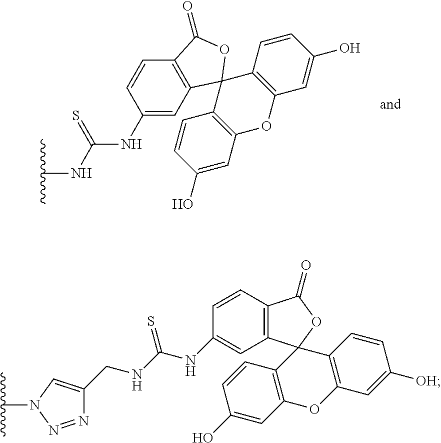 [SMILES: *NC(=S)Nc1ccc2c(c1)C1(OC2=O)c2ccc(O)cc2Oc2cc(O)ccc21 ; *n1cc(CNC(=S)Nc2ccc3c(c2)C2(OC3=O)c3ccc(O)cc3Oc3cc(O)ccc32)nn1]

and

each R₇ is independently H, Na, K, optionally substituted alkyl, optionally substituted aryl, or optionally substituted arylalkyl. In another aspect, the compound of Formula IV is represented by Formula VI:

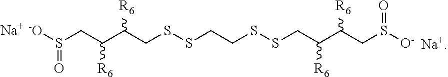

Other aspects and embodiments of the invention are disclosed infra.

### BRIEF DESCRIPTION OF THE DRAWINGS

The present invention is further described below with reference to the following non-limiting examples and with reference to the following figures, in which:

FIG. 1. depicts X-ray crystal structures of the extracellular domains of EGFR, HER2, and HER3 with cysteine residues shown in red. Note the large number of disulfide bonds.

FIG. 2. depicts 2A) Photomicrographs of MDA-MB-468 or BxPC3 cells treated for 24 hours with 25 μM NSC624205 or the vehicle control. 2B) MDA-MB-468 cells were treated for 24 hours with the indicated concentrations of NSC624203, NSC624204, and NSC624205 and cell viability (mass) was measured by crystal violet staining. 2C) Photomicrographs of MDA-MB-468, SKBR3, or MDA-MB-231 cells treated for 24 h with 10 μM NSC624205 or the vehicle control. 2D) Cell proliferation was measured by thymidine incorporation through the treatment of MDAMB-468 and SKBR3 cells for 24 hours with NSC624203, an EGFR/HER2 inhibitor, or a combination of the two compounds. Results are presented as the average of triplicate determinations±S.D. 2E) The indicated cancer cell lines were treated for 24 h with 20 μM NSC624205 or vehicle and cell extracts were analyzed by immunoblot. Actin serves as a loading control.

FIG. 3. depicts 3A) the analysis of MDA-MB-468 cells treated as indicated for 24 h by immunoblot for levels of EGFR and EGFR phosphorylation. 3B) the analysis of MDA-MB-468 cells treated as indicated for 24 h by immunoblot for PARP cleavage. 3C) MDA-MB-468 cells were either left untreated, or treated with 20 μM NSC624205 for 24 hours. NSC624205-treated cells were then washed and incubated for the indicated periods in the absence of drug. EGFR electrophoretic mobility was analyzed by immunoblot. 3D) MDA-MB-468 cells were pretreated with 25 μM NSC624205 or vehicle for 15 hours and then either left untreated or stimulated for 15 minutes with 20 ng/ml EGF, after which cell extracts were analyzed by immunoblot. 3E) MDA-MB-468 cells were treated as indicated for 24 hours and analyzed by immunoblot.

FIG. 4. depicts 4A) the treatment of vector control or EGFR overexpressing T47D cells with 20 μM NSC624205 or vehicle for 24 hours and then photographed. Extensive cell death was observed in the T47D.EGFR cells, but not the T47D.Vector cells. 4B) Cells treated as in 4A) were subjected to immunoblot analysis. 4C) Thymidine incorporation measured as in FIG. 2D of vector control (T47D.Puro) or EGFR overexpressing (T47D.EGFR) cells treated for 24 h with increasing concentrations of NSC624203 or LY294002. p values were calculated using Student's unpaired t-test.

FIG. 5. depicts 5A) Photomicrographs of MDA-MB-468 cells treated for 24 h with 20 μM of the indicated compounds. 5B) Immunoblot analysis of MDA-MB-468 cells treated as in 5A. 5C) Chemical structures of Disulfide bond Disrupting Agents (DDAs) showing active compounds on the left side with the pharmacophore highlighted in red, along with the generic pharmacophore. Inactive compounds either lack sulfinate or disulfide groups, or do not have the appropriate four-carbon “spacer” between these groups. The exception to this rule is NSC627175/DTDO, which represents a different pharmacophore. 5D) Viability of BT474 or MDA-MB-468 cells treated for 24 h with the indicated drug at the specified concentrations was measured in MTT assays. Assays were carried out in triplicate and results were presented as the average±S.D. 5E) Proliferation of tert-immortalized human mammary epithelial cells (HMEC-tert) and MDA-MB-468, BT474, and SKBR3 breast cancer cells after incubation with the indicated concentrations of RBF3 for 24 h was measured in thymidine incorporation assays as described in FIG. 2D. 5F)-5H) The indicated cell lines were treated with the indicated compounds at 20 μM unless otherwise indicated for 24 h and analyzed by immunoblot.

FIG. 6. depicts 6A) Proposed model for how DDAs disrupt disulfide bonds by either inserting into them (a) or changing their connectivity (b). 6C) Proposed reactions based on the reaction products identified by mass spectrometry.

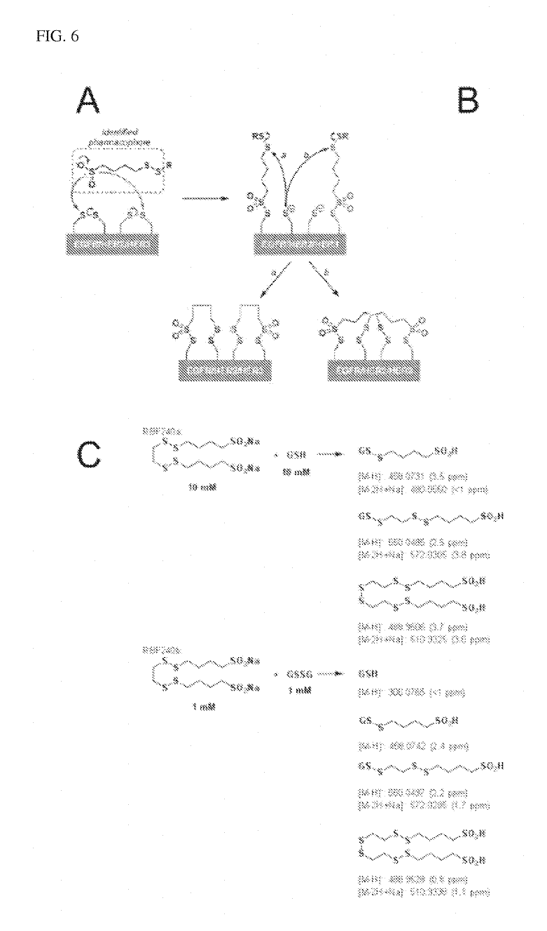

FIG. 7. depicts 7A) Growth of tumors derived from BT474 cells in mice treated with either Vehicle (water; red lines) or 40 mg/kg RBF3 (blue lines). Animals were treated by intraperitoneal injections administered once daily, Monday-Friday. 7B) Plot of animal weights over time. 7C) Photomicrographs of hematoxylin and eosin (H&E) stained sections of tumors from vehicle- or RBF3-treated mice. Pictures of normal tissues (brain, lung, liver, kidney) and tumor tissues from mice treated with vehicle or 160 mg/kg RBF3. Note the presence of extensive necrosis in the RBF3-treated tumors.

FIG. 8. depicts 8A) Viability of HCC1954 cells treated as indicated for 24 h measured in MTT assays. 8B) Photomicrographs of HCC1954 cells treated for 24 h with vehicle (Control) or 20 μM RBF3, 100 nM Rapamycin, or 20 μM Lapatinib either alone or in pairwise combinations. C. Immunoblot analysis of HCC1954 cells treated as in 8B.

### DETAILED DESCRIPTION OF THE INVENTION

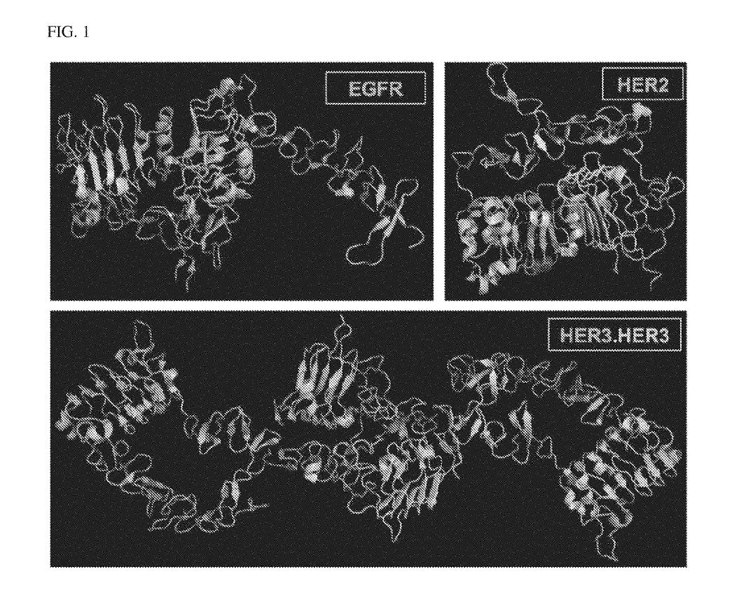

EGFR, HER2, and HER3 share evolutionarily conserved extracellular domains stabilized by disulfide bonds (FIG. 1) [Ogiso, H., Ishitani, R., Nureki, O., Fukai, S., Yamanaka, M., Kim, J. H., Saito, K., Sakamoto, A., Inoue, M., Shirouzu, M., and Yokoyama, S. (2002) Crystal structure of the complex of human epidermal growth factor and receptor extracellular domains Cell 110, 775-787; Garrett, T. P., McKern, N. M., Lou, M., Elleman, T. C., Adams, T. E., Lovrecz, G. O., Kofler, M., Jorissen, R. N., Nice, E. C., Burgess, A. W., and Ward, C. W. (2003) The crystal structure of a truncated ErbB2 ectodomain reveals an active conformation, poised to interact with other ErbB receptors Mol Cell 11, 495-505; Cho, H. S., Mason, K., Ramyar, K. X., Stanley, A. M., Gabelli, S. B., Denney, D. W., Jr., and Leahy, D. J. (2003) Structure of the extracellular region of HER2 alone and in complex with the Herceptin Fab Nature 421, 756-760; Cho, H. S., and Leahy, D. J. (2002) Structure of the extracellular region of HER3 reveals an interdomain tether Science 297, 1330-13331. Given the intricate and extensive network of disulfide bonding in these receptors, compounds able to disrupt disulfide bonds (e.g., any of the compounds herein or formulae presented herein) would preferentially inactivate these oncogenic proteins.

### 1. DEFINITIONS

Before further description of the present invention, and in order that the invention may be more readily understood, certain terms are first defined and collected here for convenience.

The term “administration” or “administering” includes routes of introducing the compound of the invention(s) to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally affect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a pro-drug form which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C1-C30 for straight chain, C₃-C₃₀ for branched chain), preferably 26 or fewer, and more preferably 20 or fewer, and still more preferably 4 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and sentences is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and still more preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In certain embodiments, the term “lower alkyl” includes a straight chain alkyl having 4 or fewer carbon atoms in its backbone, e.g., C1-C4 alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “biological activities” of a compound of the invention includes all activities elicited by compound of the inventions in a responsive cell. It includes genomic and non-genomic activities elicited by these compounds.

“Biological composition” or “biological sample” refers to a composition containing or derived from cells or biopolymers. Cell-containing compositions include, for example, mammalian blood, red cell platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, saliva, placental extracts, a cryoprecipitate, a cryosupernatant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In one embodiment, a suitable biological composition or biological sample is a red blood cell suspension. In some embodiments, the blood cell suspension includes mammalian blood cells. Preferably, the blood cells are obtained from a human, a non-human primate, a dog, a cat, a horse, a cow, a goat, a sheep or a pig. In certain embodiments, the blood cell suspension includes red blood cells and/or platelets and/or leukocytes and/or bone marrow cells.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “effective amount” includes an amount effective, at dosages and for periods of time necessary, to achieve the desired result, e.g., sufficient to treat a cell proliferative disorder. An effective amount of compound of the invention may vary according to factors such as the disease state, age, and weight of the subject, and the ability of the compound of the invention to elicit a desired response in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

A therapeutically effective amount of compound of the invention (i.e., an effective dosage) may range from about 0.001 to 30 mg/kg body weight, or about 0.01 to 25 mg/kg body weight, or about 0.1 to 20 mg/kg body weight, or about 1 to 10 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or can include a series of treatments. In one example, a subject is treated with a compound of the invention in the range of between about 0.1 to 20 mg/kg body weight, one time per week for between about 1 to 10 weeks, or between 2 to 8 weeks, or between about 3 to 7 weeks, or for about 4, 5, or 6 weeks. It will also be appreciated that the effective dosage of a compound of the invention used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “improved biological properties” refers to any activity inherent in a compound of the invention that enhances its effectiveness in vivo. In certain embodiments, this term refers to any qualitative or quantitative improved therapeutic property of a compound of the invention, such as reduced toxicity.

The term “cell proliferative disorder” includes disorders involving the undesired or uncontrolled proliferation of a cell. Examples of such disorders include, but are not limited to, tumors (e.g., brain, lung (small cell and non-small cell), ovary, prostate, breast or colon) or other carcinomas or sarcomas (e.g., leukemia, lymphoma).

The term “optionally substituted” is intended to encompass groups that are unsubstituted or are substituted by other than hydrogen at one or more available positions, typically 1, 2, 3, 4 or 5 positions, by one or more suitable groups (which may be the same or different). Such optional substituents include, for example, hydroxy, halogen, cyano, nitro, C₁-C₈alkyl, C₂-C₈ alkenyl, C₂-C₈alkynyl, C₁-C₈alkoxy, C₂-C₈alkyl ether, C₃-C₈alkanone, C₁-C₈alkylthio, amino, mono- or di-(C1-C₈alkyl)amino, haloC₁-C₈alkyl, haloC₁-C₈alkoxy, C₁-C₈alkanoyl, C₂-C₈alkanoyloxy, C₁-C₈alkoxycarbonyl, —COOH, —CONH₂, mono- or di-(C₁-C₈alkyl)aminocarbonyl, —SO₂NH₂, and/or mono or di(C₁-C₈alkyl)sulfonamido, as well as carbocyclic and heterocyclic groups. Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substituents).

The term “isomers” or “stereoisomers” refers to compounds which have identical chemical constitution, but differ with regard to the arrangement of the atoms or groups in space.

The term “modulate” refers to an increase or decrease, e.g., in the ability of a cell to proliferate in response to exposure to a compound of the invention, e.g., the inhibition of proliferation of at least a sub-population of cells in an animal such that a desired end result is achieved, e.g., a therapeutic result.

The term “obtaining” as in “obtaining a compound capable of inhibiting CDCP1” is intended to include purchasing, synthesizing or otherwise acquiring the compound.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” or “pro-drug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective amount” of a compound refers to an amount of a compound of the invention any formula herein or otherwise described herein which is effective, upon single or multiple dose administration to the patient, in preventing or treating a cell proliferative disorder.

The language “reduced toxicity” is intended to include a reduction in any undesired side effect elicited by a compound of the invention when administered in vivo.

The term “sulfhydryl” or “thiol” means —SH.

The term “subject” includes organisms which are capable of suffering from a cell proliferative disorder or who could otherwise benefit from the administration of a compound of the invention, such as human and non-human animals. Preferred humans include human patients suffering from or prone to suffering from a cell proliferative disorder or associated state, as described herein. The term “non-human animals” of the invention includes all vertebrates, e.g., mammals; e.g., rodents; e.g., mice; and non-mammals, such as non-human primates; e.g., sheep, dog, cow, chickens, amphibians, reptiles, etc.

The term “susceptible to a cell proliferative disorder” is meant to include subjects at risk of developing disorder of cell proliferation, e.g., cancer, i.e., subjects suffering from viral infection with cancer causing viruses, subjects that have been exposed to ionizing radiation or carcinogenic compounds, subjects having a family or medical history of cancer, and the like.

The phrases “systemic administration,” “administered systemically”, “peripheral administration” and “administered peripherally” as used herein mean the administration of a compound of the invention(s), drug or other material, such that it enters the patient's system and, thus, is subject to metabolism and other like processes, for example, subcutaneous administration.

The language “therapeutically effective amount” of a compound of the invention refers to an amount of an agent which is effective, upon single or multiple dose administration to the patient, in inhibiting cell proliferation and/or symptoms of a cell proliferative disorder, or in prolonging the survivability of the patient with such a cell proliferative disorder beyond that expected in the absence of such treatment.

With respect to the nomenclature of a chiral center, terms “d” and “1” configuration are as defined by the IUPAC Recommendations. As to the use of the terms, diastereomer, racemate, epimer and enantiomer will be used in their normal context to describe the stereochemistry of preparations.

### 2. COMPOUNDS OF THE INVENTION

In one aspect, the invention provides a compound that inhibits or is capable of inhibiting EGFR, HER2, and/or HER3. In another aspect, the compound inhibits or is capable of inhibiting at least two of EGFR, HER2, and HER3. In another aspect, the compound inhibits or is capable of inhibiting all three of EGFR, HER2, and HER3. In another aspect, the compound is capable of treating HER2-positive breast cancer. In another aspect, the compound is capable of treating breast cancer modulated by EGFR, HER2, and/or HER3.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. USES OF THE COMPOUNDS OF THE INVENTION

As described herein below, it has now surprisingly been found that the compounds of the invention and analogs can inactivate EGFR, HER2, and/or HER3, and thereby treat disorders of cell proliferation, including cancer. Thus, compounds of the invention overcome the deficiencies of treating breast cancer with HER2-targeted antibodies (e.g., Trastuzumab and Pertuzumab), which only specifically target the single receptor, HER2, to which 66-88% of HER2-positive tumors exhibit primary resistance.

Thus, in one embodiment, the invention provides methods for treating a subject for a cell proliferative disorder, by administering to the subject an effective amount of a compound of the invention (e.g., a compound of any formula herein or otherwise described herein). A cell proliferative disorder includes cancer. In certain embodiments, the subject is a mammal, e.g., a primate, e.g., a human.

A further aspect presents a method of treating a subject suffering from or susceptible to cancer, including administering to the subject an effective amount of a compound of the invention (e.g., a compound of any formula herein or otherwise described herein) to thereby treat the subject suffering from or susceptible to cancer.

In certain embodiments, the methods of the invention include administering to a subject a therapeutically effective amount of a compound of the invention in combination with another pharmaceutically active compound. Examples of pharmaceutically active compounds include compounds known to treat cell proliferative disorders, e.g., imatinib (Gleevec). Other pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell New Jersey, Medical Economics Co., the complete contents of which are expressly incorporated herein by reference. The compound of the invention and the pharmaceutically active compound may be administered to the subject in the same pharmaceutical composition or in different pharmaceutical compositions (at the same time or at different times).

In certain embodiments, the compound of the invention can be used in combination therapy with conventional cancer chemotherapeutics. Conventional treatment regimens for leukemia and for other tumors include radiation, surgery, drugs, or combinations thereof. In addition to radiation, the following drugs, usually in combinations with each other, are often used to treat acute leukemias: vincristine, prednisone, methotrexate, mercaptopurine, cyclophosphamide, and cytarabine. In chronic leukemia, for example, busulfan, melphalan, and chlorambucil can be used in combination. Most conventional anti-cancer drugs are highly toxic and tend to make patients quite ill while undergoing treatment. Vigorous therapy is based on the premise that unless every cancerous cell is destroyed, the residual cells will multiply and cause a relapse.

Determination of a therapeutically effective anti-proliferative amount or a prophylactically effective anti-proliferative amount of the compound of the invention of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective anti-proliferative amount or dose, and the prophylactically effective anti-proliferative amount or dose, a number of factors are considered by the attending clinician, including, but not limited to: the specific cell proliferative disorder involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the invention with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective anti-proliferative amount of a compound of the invention of the invention is expected to vary from about 0 1 milligram per kilogram of body weight per day (mg/kg/day) to about 100 mg/kg/day.

Compounds determined to be effective for the prevention or treatment of cell proliferative disorders in animals, e.g., dogs, chickens, and rodents, may also be useful in treatment of tumors in humans. Those skilled in the art of treating tumors in humans will know, based upon the data obtained in animal studies, the dosage and route of administration of the compound to humans. In general, the dosage and route of administration in humans is expected to be similar to that in animals.

The identification of those patients who are in need of prophylactic treatment for cell proliferative disorders is well within the ability and knowledge of one skilled in the art. Certain of the methods for identification of patients which are at risk of developing cell proliferative disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination and medical/family history.

A method of assessing the efficacy of a treatment in a subject includes determining the pre-treatment extent of a cell proliferative disorder by methods well known in the art (e.g., determining tumor size or screening for tumor markers where the cell proliferative disorder is cancer) and then administering a therapeutically effective amount of an inhibitor of cell proliferation (e.g., a compound of any formula herein or otherwise described herein) according to the invention to the subject. After an appropriate period of time after the administration of the compound (e.g., 1 day, 1 week, 2 weeks, one month, six months), the extent of the cell proliferative disorder is determined again. The modulation (e.g., decrease) of the extent or invasiveness of the cell proliferative disorder indicates efficacy of the treatment. The extent or invasiveness of the cell proliferative disorder may be determined periodically throughout treatment. For example, the extent or invasiveness of the cell proliferative disorder may be checked every few hours, days or weeks to assess the further efficacy of the treatment. A decrease in extent or invasiveness of the cell proliferative disorder indicates that the treatment is efficacious. The method described may be used to screen or select patients that may benefit from treatment with an inhibitor of a cell proliferative disorder.

As used herein, “obtaining a biological sample from a subject,” includes obtaining a sample for use in the methods described herein. A biological sample is described above.

Yet another aspect presents a method to identify a compound that inhibits cell proliferation by measuring the compound's ability to inhibit or inactivate EGFR, HER2, and/or HER3. The method may include utilizing a homology model of EGFR, HER2, and/or HER3. Compounds may be computer modeled into or on a EGFR, HER2, and/or HER3 binding site of the homology model to identify EGFR, HER2, and/or HER3 inhibitory compounds. Once potential inhibitory compounds are identified, the compounds may be screened using cellular assays, such as the ones identified below in the Examples and competition assays known in the art. Compounds identified that affect EGFR, HER2, and/or HER3 signaling could be inhibitors or activators (more preferably inhibitors) of EGFR, HER2, and/or HER3 binding and could be useful therapeutic agents.

According to another aspect, the invention provides methods for designing, evaluating and identifying compounds which bind to EGFR, HER2, and/or HER3 binding pockets. These methods involve the use of a three-dimensional graphical structure of a molecule or a molecular complex which comprises a binding site (e.g., a binding site in EGFR, HER2, and/or HER3). Such compounds are potential inhibitors of EGFR, HER2, and/or HER3.

Structure data, when used in conjunction with a computer programmed with software to translate those coordinates into the 3-dimensional structure of a molecule or molecular complex comprising a binding pocket may be used for a variety of purposes, such as drug discovery.

For example, the structure encoded by the data may be computationally evaluated for its ability to associate with chemical entities. Chemical entities that associate with a binding site of EGFR, HER2, and/or HER3 may inhibit EGFR, HER2, and/or HER3 or EGFR, HER2, and/or HER3 signaling, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention relates to a method for evaluating the potential of a chemical entity to associate with a molecule or molecular complex comprising a binding pocket defined by structure coordinates of EGFR, HER2, and/or HER3.

This method comprises the steps of:

i) employing computational means to perform a fitting operation between the chemical entity and a binding pocket of the molecule or molecular complex (e.g., a binding site in EGFR, HER2, and/or HER3); and

ii) analyzing the results of the fitting operation to quantify the association between the chemical entity and the binding pocket. This embodiment relates to evaluating the potential of a chemical entity to associate with or bind to a binding site in EGFR, HER2, and/or HER3.

The term “chemical entity”, as used herein, refers to chemical compounds, complexes of at least two chemical compounds, and fragments of such compounds or complexes.

In certain embodiments, the method evaluates the potential of a chemical entity to associate with a molecule or molecular complex defined by structure coordinates of all of the amino acids of EGFR, HER2, and/or HER3, as described herein, or a homologue of said molecule or molecular complex.

In a further embodiment, the structural coordinates of one of the binding pockets described herein can be utilized in a method for identifying a potential agonist or antagonist of EGFR, HER2, and/or HER3. This method comprises the steps of:

a) using the atomic coordinates of EGFR, HER2, and/or HER3 protein (e.g., a binding site in EGFR, HER2, and/or HER3) to generate a three-dimensional structure of EGFR, HER2, and/or HER3 (e.g., a binding site in EGFR, HER2, and/or HER3);

b) employing the three-dimensional structure to design or select the potential agonist or antagonist. The method further includes the optional steps of c) synthesizing the agonist or antagonist; and d) contacting the agonist or antagonist with EGFR, HER2, and/or HER3, or homologue thereof, or antagonist to interact with EGFR, HER2, and/or HER3, or homologue thereof.

The design of compounds that bind to or inhibit EGFR, HER2, and/or HER3 binding sites (e.g., a binding site in EGFR, HER2, and/or HER3) according to this invention generally involves consideration of several factors. First, the entity may physically and structurally associate with parts or all of the EGFR, HER2, and/or HER3 binding sites (e.g., a binding site in EGFR, HER2, and/or HER3). Non-covalent molecular interactions important in this association include hydrogen bonding, van der Waals interactions, hydrophobic interactions and electrostatic interactions. Second, the entity may assume a conformation that allows it to associate with the EGFR, HER2, and/or HER3 binding sites (e.g., a binding site in EGFR, HER2, and/or HER3) directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket(s), or the spacing between functional groups of an entity comprising several chemical entities that directly interact with the binding pocket or homologues thereof.

The potential inhibitory or binding effect of a chemical entity on EGFR, HER2, and/or HER3 binding sites (e.g., a binding site in EGFR, HER2, and/or HER3) may be analyzed prior to its actual synthesis and testing by the use of computer modeling techniques. If the theoretical structure of the given entity suggests insufficient interaction and association between it and the target binding pocket, testing of the entity is obviated. However, if computer modeling indicates a strong interaction, the molecule may then be synthesized and tested for its ability to bind to a binding site. This may be achieved, e.g., by testing the ability of the molecule to inhibit EGFR, HER2, and/or HER3, e.g., using assays described herein or known in the art. In this manner, synthesis of inoperative compounds may be avoided.

A potential inhibitor of EGFR, HER2, and/or HER3 binding sites (e.g., a binding site in EGFR, HER2, and/or HER3) may be computationally evaluated by means of a series of steps in which chemical entities or fragments are screened and selected for their ability to associate with the EGFR, HER2, and/or HER3 binding sites (e.g., a binding site in EGFR, HER2, and/or HER3).

One skilled in the art may use one of several methods to screen chemical entities or fragments for their ability to associate with EGFR, HER2, and/or HER3 binding sites (e.g., a binding site in EGFR, HER2, and/or HER3). This process may begin by visual inspection of, for example, a EGFR, HER2, and/or HER3 binding site (e.g., a binding site in EGFR, HER2, and/or HER3) on the computer screen based on the EGFR, HER2, and/or HER3 structure coordinates described herein, or other coordinates which define a similar shape generated from the machine-readable storage medium. Selected fragments or chemical entities may then be positioned in a variety of orientations, or docked, within that binding site as defined supra. Docking may be accomplished using software such as Quanta and DOCK, followed by energy minimization and molecular dynamics with standard molecular mechanics force fields, such as CHARMM and AMBER.

Specialized computer programs (e.g., as known in the art and/or commercially available and/or as described herein) may also assist in the process of selecting fragments or chemical entities.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target binding site.

Instead of proceeding to build an inhibitor of a binding pocket in a step-wise fashion one fragment or chemical entity at a time as described above, inhibitory or other binding compounds may be designed as a whole or “de novo” using either an empty binding site or optionally including some portion(s) of a known inhibitor(s). There are many de novo ligand design methods known in the art, some of which are commercially available (e.g., LeapFrog, available from Tripos Associates, St. Louis, Mo.).

Other molecular modeling techniques may also be employed (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)).

Once a compound has been designed or selected, the efficiency with which that entity may bind to a binding pocket may be tested and optimized by computational evaluation.

Specific computer software is available in the art to evaluate compound deformation energy and electrostatic interactions. Examples of programs designed for such uses include: AMBER; QUANTA/CHARMM (Accelrys, Inc., Madison, Wis.) and the like. These programs may be implemented, for instance, using a commercially-available graphics workstation. Other hardware systems and software packages will be known to those skilled in the art. Another technique involves the in silico screening of virtual libraries of compounds, e.g., as described herein. Many thousands of compounds can be rapidly screened and the best virtual compounds can be selected for further screening (e.g., by synthesis and in vitro testing). Small molecule databases can be screened for chemical entities or compounds that can bind, in whole or in part, to EGFR, HER2, and/or HER3 binding sites (e.g., a binding site in EGFR, HER2, and/or HER3). In this screening, the quality of fit of such entities to the binding site may be judged either by shape complementarity or by estimated interaction energy.

In another aspect, a compound of the invention is packaged in a therapeutically effective amount with a pharmaceutically acceptable carrier or diluent. The composition may be formulated for treating a subject suffering from or susceptible to a cell proliferative disorder, and packaged with instructions to treat a subject suffering from or susceptible to a cell proliferative disorder.

In another aspect, the invention provides methods for inhibiting cell proliferation. In one embodiment, a method of inhibiting cell proliferation (or a cell proliferative disorder) according to the invention includes contacting cells with a compound capable of inhibiting EGFR, HER2, and/or HER3 signaling. In another embodiment, a method of inhibiting cell proliferation (or a cell proliferative disorder) according to the invention includes contacting cells with a compound capable of inhibiting EGFR, HER2, and/or HER3 signaling in the cells. In either embodiment, the contacting may be in vitro, e.g., by addition of the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

In another aspect, methods of inhibiting a cell proliferative disorder in a subject include administering an effective amount of a compound of the invention to the subject. The administration may be by any route of administering known in the pharmaceutical arts. The subject may have a cell proliferative disorder, may be at risk of developing a cell proliferative disorder, or may need prophylactic treatment prior to anticipated or unanticipated exposure to conditions capable of increasing susceptibility to a cell proliferative disorder, e.g., exposure to carcinogens or to ionizing radiation.

In one aspect, a method of monitoring the progress of a subject being treated with a compound capable of inhibiting EGFR, HER2, and/or HER3 includes determining the pre-treatment status (e.g., size, growth rate, or invasiveness of a tumor) of the cell proliferative disorder, administering a therapeutically effective amount of a EGFR, HER2, and/or HER3 inhibitor to the subject, and determining the status of the cell proliferative disorder after an initial period of treatment with the EGFR, HER2, and/or HER3 inhibitor, wherein the modulation of the status indicates efficacy of the treatment.

In one aspect, a method of monitoring the progress of a subject being treated with a compound capable of inhibiting EGFR, HER2, and/or HER3 signaling includes determining the pre-treatment status (e.g., size, growth rate, or invasiveness of a tumor) of the cell proliferative disorder, administering a therapeutically effective amount of a compound capable of inhibiting EGFR, HER2, and/or HER3 signaling to the subject, and determining the status (e.g., size, growth rate, or invasiveness of a tumor) of the cell proliferative disorder after an initial period of treatment with the compound capable of inhibiting EGFR, HER2, and/or HER3 signaling, wherein the modulation of the status indicates efficacy of the treatment.

In one aspect, a method of monitoring the progress of a subject being treated with a compound capable of inhibiting EGFR, HER2, and/or HER3 signaling includes determining the pre-treatment status (e.g., size, growth rate, or invasiveness of a tumor) of the cell proliferative disorder, administering a therapeutically effective amount of a compound capable of inhibiting EGFR, HER2, and/or HER3 signaling to the subject, and determining the status (e.g., size, growth rate, or invasiveness of a tumor) of the cell proliferative disorder after an initial period of treatment with the compound capable of inhibiting EGFR, HER2, and/or HER3 signaling, wherein the modulation of status is an indication that the cell proliferative disorder is likely to have a favorable clinical response to treatment with a compound capable of inhibiting EGFR, HER2, and/or HER3 signaling.

The subject may be at risk of a cell proliferative disorder, may be exhibiting symptoms of a cell proliferative disorder, may be susceptible to a cell proliferative disorder and/or may have been diagnosed with a cell proliferative disorder.

The initial period of treatment may be the time in which it takes to establish a stable and/or therapeutically effective blood serum level of the compound capable of inhibiting EGFR, HER2, and/or HER3 signaling, or the time in which it take for the subject to clear a substantial portion of the compound, or any period of time selected by the subject or healthcare professional that is relevant to the treatment.

If the modulation of the status indicates that the subject may have a favorable clinical response to the treatment, the subject may be treated with the compound. For example, the subject can be administered a therapeutically effective dose or doses of the compound.

In another aspect, the invention provides methods for inhibiting EGFR, HER2, and/or HER3 signaling in a cell. The methods include contacting the cell with an effective amount of a compound capable of inhibiting EGFR, HER2, and/or HER3 signaling, such that the signaling of EGFR, HER2, and/or HER3 is reduced The contacting may be in vitro, e.g., by addition of the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

In another aspect, the invention provides methods for identifying an inhibitor of EGFR, HER2, and/or HER3. The methods include contacting EGFR, HER2, and/or HER3 with a compound capable of inhibiting EGFR, HER2, and/or HER3, such that the signaling of EGFR, HER2, and/or HER3 is inhibited.

The EGFR, HER2, and/or HER3 may be within a cell, isolated from a cell, recombinantly expressed, purified or isolated from a cell or recombinant expression system or partially purified or isolated from a cell or recombinant expression system.

The contacting may be in vitro, e.g., by addition of the compound to a solution containing purified EGFR, HER2, and/or HER3, or, if EGFR, HER2, and/or HER3 is present in cells, by adding the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

Kits of the invention include kits for treating a cell proliferative disorder in a subject. The invention also includes kits for downregulating expression of EGFR, HER2, and/or HER3, stabilizing an interaction of EGFR, HER2, and/or HER3, assessing the efficacy of a treatment for a cell proliferative disorder in a subject, monitoring the progress of a subject being treated for a cell proliferative disorder, selecting a subject with a cell proliferative disorder for treatment according to the invention, and/or treating a subject suffering from or susceptible to a cell proliferative disorder. The kit may include a compound of the invention (e.g., a compound of any formula herein or otherwise described herein) and instructions for use. The instructions for use may include information on dosage, method of delivery, storage of the kit, etc. The kits may also include reagents, for example, test compounds, buffers, media (e.g., cell growth media), cells, etc. Test compounds may include known compounds or newly discovered compounds, for example, combinatorial libraries of compounds. One or more of the kits of the invention may be packaged together, for example, a kit for assessing the efficacy of a treatment for a cell proliferative disorder may be packaged with a kit for monitoring the progress of a subject being treated for a cell proliferative disorder according to the invention.

The present methods can be performed on cells in culture, e.g. in vitro or ex vivo, or on cells present in an animal subject, e.g., in vivo. Compounds of the inventions can be initially tested in vitro using primary cultures of proliferating cells, e.g., transformed cells, tumor cell lines, and the like.

Alternatively, the effects of compound of the invention can be characterized in vivo using animals models.

### 4. PHARMACEUTICAL COMPOSITIONS

The invention also provides a pharmaceutical composition, comprising an effective amount of a compound of the invention (e.g., a compound capable of inhibiting EGFR, HER2, and/or HER3, a compound capable of stabilizing the interaction between the compound and EGFR, HER2, and/or HER3, or a compound of any formula herein or otherwise described herein) and a pharmaceutically acceptable carrier. In a further embodiment, the effective amount is effective to treat a cell proliferative disorder, as described previously.

In an embodiment, the compound of the invention is administered to the subject using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compound of the inventions of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present. Solubilizing agents, including for example, cremaphore and beta-cyclodextrins can also used in the pharmaceutical compositions herein.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The invention also provides a pharmaceutical composition, comprising an effective amount of a compound described herein and a pharmaceutically acceptable carrier. In an embodiment, compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

By “pharmaceutically effective amount” as used herein is meant an amount of a compound of the invention, high enough to significantly positively modify the condition to be treated but low enough to avoid serious side effects (at a reasonable benefit/risk ratio), within the scope of sound medical judgment. A pharmaceutically effective amount of a compound of the invention will vary with the particular goal to be achieved, the age and physical condition of the patient being treated, the severity of the underlying disease, the duration of treatment, the nature of concurrent therapy and the specific compound employed. For example, a therapeutically effective amount of a compound of the invention administered to a child or a neonate will be reduced proportionately in accordance with sound medical judgment. The effective amount of a compound of the invention will thus be the minimum amount which will provide the desired effect.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound of the invention(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, or from about 5 percent to about 70 percent, or from about 10 percent to about 30 percent.

Methods of preparing these compositions include the step of bringing into association a compound of the invention(s) with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound of the invention(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound of the invention(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound of the invention(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound of the invention(s) include powders, sprays, ointments, pastes, creams, lotions, gels, solutions, patches and inhalants. The active compound of the invention(s) may be mixed under sterile conditions with a pharmaceutically-acceptable carrier, and with any preservatives, buffers, or propellants which may be required.

The ointments, pastes, creams and gels may contain, in addition to compound of the invention(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the invention(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound of the invention(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the invention(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise one or more compound of the invention(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound of the invention(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound of the invention(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound of the invention(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the compound of the invention for the present invention is the maximum that a patient can tolerate and not develop serious side effects. Preferably, the compound of the invention of the present invention is administered at a concentration of about 0.001 mg to about 100 mg per kilogram of body weight, about 0.001-about 10 mg/kg or about 0.001 mg-about 100 mg/kg of body weight. Ranges intermediate to the above-recited values are also intended to be part of the invention.

For nasal administration or administration by inhalation or insufflation, the active compound(s) or prodrug(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

A specific example of an aqueous suspension formulation suitable for nasal administration using commercially-available nasal spray devices includes the following ingredients: active compound or prodrug (0.5-20 mg/ml); benzalkonium chloride (0.1-0.2 mg/mL); polysorbate 80 (TWEEN® 80; 0.5-5 mg/ml); carboxymethylcellulose sodium or microcrystalline cellulose (1-15 mg/ml); phenylethanol (1-4 mg/ml); and dextrose (20-50 mg/ml). The pH of the final suspension can be adjusted to range from about pH 5 to pH 7, with a pH of about pH 5.5 being typical.

For prolonged delivery, the active compound(s) or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475, each of which is incorporated herein by reference in its entirety.

Alternatively, other pharmaceutical delivery systems can be employed. Liposomes and emulsions are well-known examples of delivery vehicles that can be used to deliver active compound(s) or prodrug(s). Certain organic solvents such as dimethylsulfoxide (DMSO) also can be employed.

The pharmaceutical compositions can, if desired, be presented in a pack or dispenser device which can contain one or more unit dosage forms containing the active compound(s). The pack can, for example, comprise metal or plastic foil, such as a blister pack. The pack or dispenser device can be accompanied by instructions for administration.

The active compound(s) or prodrug(s) of the presently disclosed subject matter, or compositions thereof, will generally be used in an amount effective to achieve the intended result, for example in an amount effective to treat or prevent the particular disease being treated. The compound(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. For example, administration of a compound to a patient suffering from an allergy provides therapeutic benefit not only when the underlying allergic response is eradicated or ameliorated, but also when the patient reports a decrease in the severity or duration of the symptoms associated with the allergy following exposure to the allergen. As another example, therapeutic benefit in the context of asthma includes an improvement in respiration following the onset of an asthmatic attack, or a reduction in the frequency or severity of asthmatic episodes. Therapeutic benefit also includes halting or slowing the progression of the disease, regardless of whether improvement is realized.

For prophylactic administration, the compound can be administered to a patient at risk of developing one of the previously described diseases. A patient at risk of developing a disease can be a patient having characteristics placing the patient in a designated group of at risk patients, as defined by an appropriate medical professional or group. A patient at risk may also be a patient that is commonly or routinely in a setting where development of the underlying disease that may be treated by administration of a metalloenzyme inhibitor according to the invention could occur. In other words, the at risk patient is one who is commonly or routinely exposed to the disease or illness causing conditions or may be acutely exposed for a limited time. Alternatively, prophylactic administration can be applied to avoid the onset of symptoms in a patient diagnosed with the underlying disorder.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC50 of the particular compound as measured in as in vitro assay, such as the in vitro fungal MIC or MFC and other in vitro assays described in the Examples section. Calculating dosages to achieve such circulating blood or serum concentrations taking into account the bioavailability of the particular compound is well within the capabilities of skilled artisans. For guidance, see Fingl & Woodbury, “General Principles,” In: Goodman and Gilman's The Pharmaceutical Basis of Therapeutics, Chapter 1, pp. 1-46, latest edition, Pagamonon Press, and the references cited therein, which are incorporated herein by reference.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

The compound(s) can be administered once per day, a few or several times per day, or even multiple times per day, depending upon, among other things, the indication being treated and the judgment of the prescribing physician.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

Another object of the present invention is the use of a compound as described herein (e.g., of any formulae herein) in the manufacture of a medicament for use in the treatment of a metalloenzyme-mediated disorder or disease. Another object of the present invention is the use of a compound as described herein (e.g., of any formulae herein) for use in the treatment of a metalloenzyme-mediated disorder or disease. Another object of the present invention is the use of a compound as described herein (e.g., of any formulae herein) in the manufacture of an agricultural composition for use in the treatment or prevention of a metalloenzyme-mediated disorder or disease in agricultural or agrarian settings.

### EXAMPLES

The invention is further illustrated by the following examples which are intended to illustrate but not limit the scope of the invention.

### Example 1: Compound Screening

EGFR, HER2, and HER3 share evolutionarily conserved extracellular domains stabilized by disulfide bonds (FIG. 1) [Ogiso, H., Ishitani, R., Nureki, O., Fukai, S., Yamanaka, M., Kim, J. H., Saito, K., Sakamoto, A., Inoue, M., Shirouzu, M., and Yokoyama, S. (2002) Crystal structure of the complex of human epidermal growth factor and receptor extracellular domains Cell 110, 775-787; Garrett, T. P., McKern, N. M., Lou, M., Elleman, T. C., Adams, T. E., Lovrecz, G. O., Kofler, M., Jorissen, R. N., Nice, E. C., Burgess, A. W., and Ward, C. W. (2003) The crystal structure of a truncated ErbB2 ectodomain reveals an active conformation, poised to interact with other ErbB receptors Mol Cell 11, 495-505; Cho, H. S., Mason, K., Ramyar, K. X., Stanley, A. M., Gabelli, S. B., Denney, D. W., Jr., and Leahy, D. J. (2003) Structure of the extracellular region of HER2 alone and in complex with the Herceptin Fab Nature 421, 756-760; Cho, H. S., and Leahy, D. J. (2002) Structure of the extracellular region of HER3 reveals an interdomain tether Science 297, 1330-13331. Given the intricate and extensive network of disulfide bonding in these receptors, it was hypothesized that compounds able to disrupt disulfide bonds (e.g., a compound of any formula herein or otherwise described herein) will preferentially inactivate these oncogenic proteins.

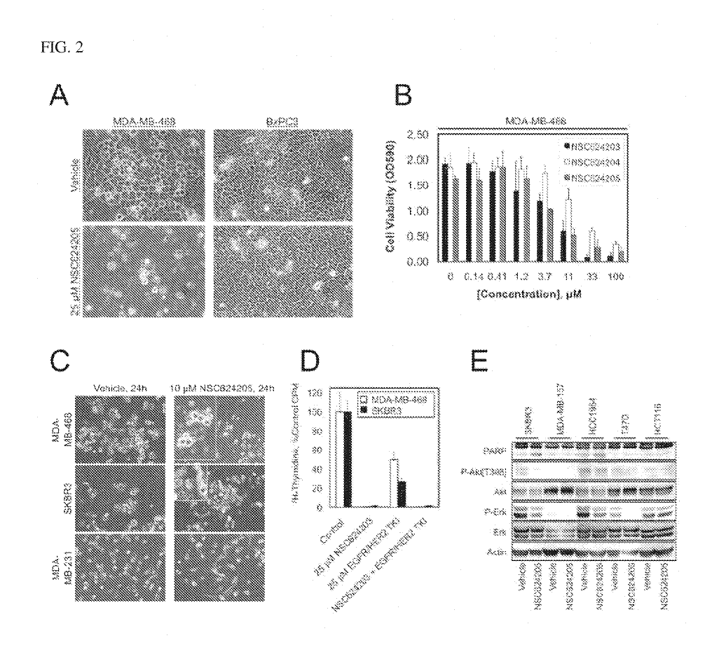

The sulfur atom in sulfinic acids can act as a nucleophile capable of breaking disulfide bonds. Therefore we obtained several sulfinate-containing compounds from the National Cancer Institute's Developmental Therapeutics Program (NCI/DTP) and, as an initial screen, examined their ability to decrease the viability of various human cancer cell lines. NSC624205 was lethal to MDA-MB-468 breast cancer cells, but had little effect on BxPC3 pancreatic cancer cells, indicating that NSC624205 is not a general cytotoxic agent (FIG. 2A). NSC624205 and two related compounds, NSC624203 and NSC624204, decreased cell viability by 50% in the range of 3.7-33 μM (FIG. 2B). Over a period of 24 hr, 10 μM NSC624205 killed MDAMB-468 cells and SKBR3 cells, which overexpress EGFR and HER2 respectively, but had little effect on the basal-like/triple-negative MDA-MB-231 breast cancer cell line, which does not overexpress either EGFR or HER2 (FIG. 2C). Comparison of the ability of NSC624203 to inhibit the proliferation of MDA-MB-468 cells with that of a commercial EGFR/HER2 tyrosine kinase inhibitor (Calbiochem, Cat. #324673) revealed that NSC624203 more effectively suppressed the proliferation of both cell lines when used at the same concentration (FIG. 2D). Examination of the effects of NSC624205 on cell signaling in a small panel of human cancer cell lines demonstrated variable effects depending on the cell line, inhibiting Akt phosphorylation in SKBR3 cells, and Erk phosphorylation in SKBR3, HCC1954, and T47D cells (FIG. 2E). Overall, cell killing by the sulfinate compounds correlated most closely with loss of Akt phosphorylation on Thr³⁰⁸.

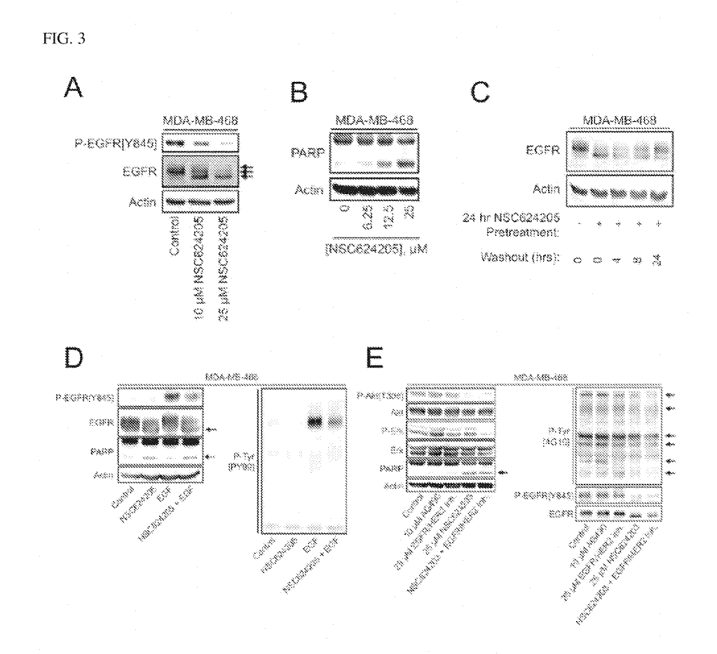

As mentioned above, it was hypothesized that sulfinate compounds may be useful in destabilizing EGFR-family members; therefore we examined the effects of NSC624205 on the levels and phosphorylation of EGFR in MDA-MB-468 cells. NSC624205 induced a concentration-dependent increase in EGFR electrophoretic mobility that correlated with a decrease in phosphorylation detected using a phospho-specific antibody (FIG. 3A). NSC624205 also caused a concentration-dependent increase in PARP cleavage, consistent with the induction of apoptosis (FIG. 3B). To examine the reversibility of NSC624205 actions, MDA-MB-468 cells were treated for 24 hr with NSC624205 and then the compound was washed out and the cells were allowed to recover for various periods of time. This experiment revealed that at 24 hours post-treatment, EGFR electrophoretic mobility was restored to near control levels, indicating that the effects of this compound are slowly reversible (FIG. 3C). To examine whether NSC624205 can suppress cellular responses to EGF, cells were stimulated with EGF either with or without NSC624205 treatment. NSC624205 decreased both overall EGF-induced cellular tyrosine phosphorylation and EGFR tyrosine phosphorylation on Tyr⁸⁴⁵ (FIG. 3D). Comparison of NSC624203 with AG490 or an EGFR/HER2 kinase inhibitor showed that NSC624203 was more effective at decreasing Akt phosphorylation, increasing PARP cleavage and reducing EGFR tyrosine phosphorylation and overall levels of cellular tyrosine phosphorylation (FIG. 3E). Interestingly, combining NSC624203 with the EGFR/HER2 inhibitor Calbiochem #324673 blocked Erk phosphorylation more effectively than either drug alone.

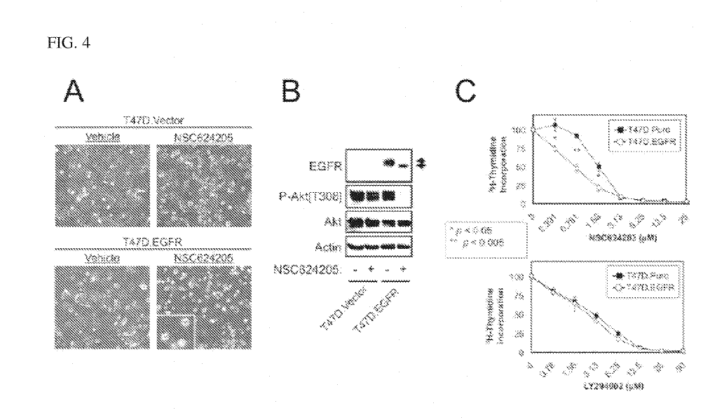

Given that T47D cells are not killed by NSC624205, we examined whether this is because these cells do not overexpress EGFR. Interestingly, T47D cells with enforced EGFR expression underwent cell death in response to NSC624205, but vector control cells did not (FIG. 4A). As observed above, NSC624205-mediated cell death correlated with an EGFR electrophoretic mobility shift, and decreased Akt phosphorylation (FIG. 4B). Cell proliferation assays showed that similarly, EGFR overexpressing T47D cells were more sensitive to NSC624203 than the control cells (FIG. 4C). However no difference between the two cell lines was observed when proliferation was suppressed using PI3-kinase inhibitor LY294002.

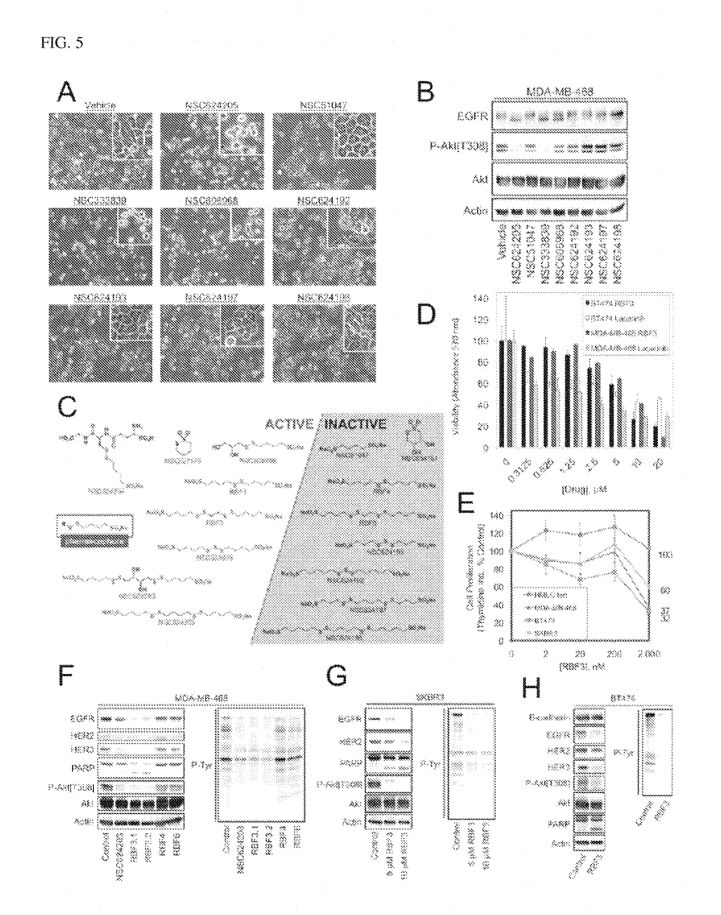

We next screened a panel of sulfinate-containing compounds that are structurally similar to NSC624203 and NSC624205. Analysis of the effects of additional NSC compounds on cell viability (FIG. 5A) or EGFR and Akt phosphorylation (FIG. 5B) demonstrated that NSC333839 has activity similar to NSC624205. NSC606968 had a partial effect on EGFR electrophoretic mobility, but only a weak effect on Akt phosphorylation. An overall evaluation of these results suggested a correlation between compound activity and the presence of a sulfinate group separated from a disulfide bond by four carbons.

### Example 2: Compound Synthesis

Additional compounds can be synthesized to determine whether compounds could be produced that had enhanced activity over the initial NSC compounds and to determine whether the sulfinate moiety is required for compound activity. These compounds can be prepared using the following four general synthetic methods.

### Method A: Synthesis of Cyclic Analogs

A solution of the appropriate dithiol or diselenol (24.7 mmol) in AcOH (25 mL) is cooled in an ice bath and a 30% aqueous H₂O₂ solution (8.8 mL) is added slowly such that the temperature does not rise above 35° C. After stirring for an appropriate amount of time, the solvent is removed under vacuum, and the residue is diluted with water (25 mL), neutralized with NaHCO₃, and extracted with toluene (4×50 mL). The organic extract is dried with MgSO₄, and the solvent is removed under vacuum. The resulting solid may be recrystallized (e.g., from Et₂O) to afford the product.

### Method B: Synthesis of Mono-Disulfide Acyclic Analogs

To a solution of the appropriate dithiane-dioxide or diselenane-dioxide (2.56 mmol) in anhydrous MeOH (6.4 mL) at room temperature (rt) under argon atmosphere, a solution of NaOMe (prepared from 58.9 mg of Na⁰ in 5.1 mL of anhydrous MeOH) is added dropwise. The mixture is stirred. The reaction mixture is then concentrated under vacuum until a precipitate is formed and acetone is then added to further facilitate the precipitation. The solid is filtered, washed with acetone (3×10 mL), and dried under reduced pressure to afford the mono-disulfide acyclic product.

### Method C: Synthesis of Poly-Disulfide Acyclic Analogs

To a mixture of the appropriate dithiane-dioxide or diselenane-dioxide (3.28 mmol) and 1,2-ethanedithiol (92 μL, 1.10 mmol) in anhydrous MeOH with stirring in ice bath, a solution of NaOMe (prepared by dissolution of 50 mg of Na0 in 2.2 mL of anhydrous MeOH) is slowly added. After the addition is complete, dry Et₂O is added to the reaction mixture until no additional precipitate is formed. The solid is filtered under vacuum and dissolved with a minimum amount of MeOH. The solution is transferred to centrifuge tubes and Et₂O is carefully added until the solution becomes turbid. The precipitate is removed by centrifugation and the supernatant is transferred to another flask, where Et₂O is added until precipitation is complete. The solid is collected by vacuum filtration and dried under reduced pressure to afford the poly-disulfide acyclic product.

### Method D: Attachment to Dyes

The compounds described herein can also be conjugated to dyes through various functionalities present in the compounds of the invention (e.g., amino, carboxylic acid, thiol, etc.) using conventional chemistries well-known in the art. As just one non-limiting example, the dyes can be attached through amino moieties on the compounds described herein by reaction with various electrophilic sources of the dyes. Examples of such electrophilic moieties are activated esters (e.g., succinimidyl esters, sulfosuccinimidyl esters, tetrafluorophenyl esters, sulfodichlorophenol esters), isothiocyanates, sulfonyl chlorides, dichlorotriazines, halides, and acyl azides. Examples of dyes are biotin, fluorescein, AlexaFluor® dyes, BODIPY®, Cascade Blue®, coumarins, Oregon Green®, Pacific Blue™ Pacific Green™, Pacific Orange™, Rhodamine Green™, Rhodamine Red™, and Texas Red®.

Compounds 10b/10b* (or mixtures thereof) can be reacted with activated esters (e.g., succinimidyl esters) of various dyes (e.g., biotin), as depicted in Scheme I, to afford the conjugated analogs (e.g., Example 11).

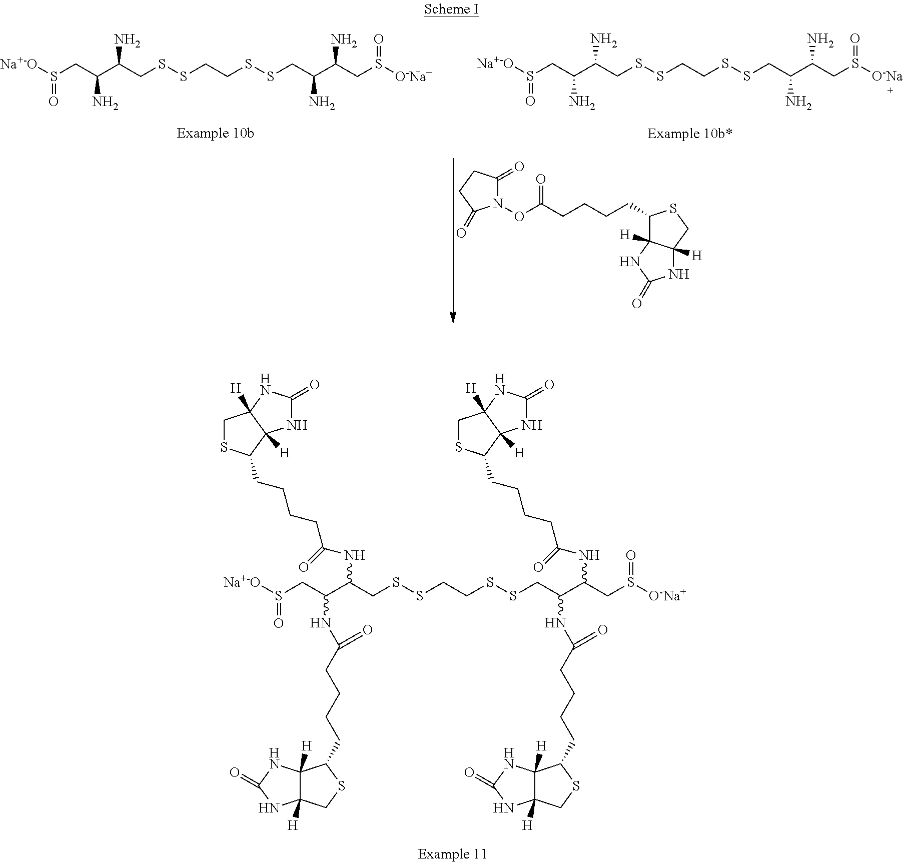

As shown in Scheme II, various dyes capped with a propargyl moiety (e.g., Biotin-NHCH2-≡) can be reacted with azides, 10c/10c*, to afford triazole, 12.

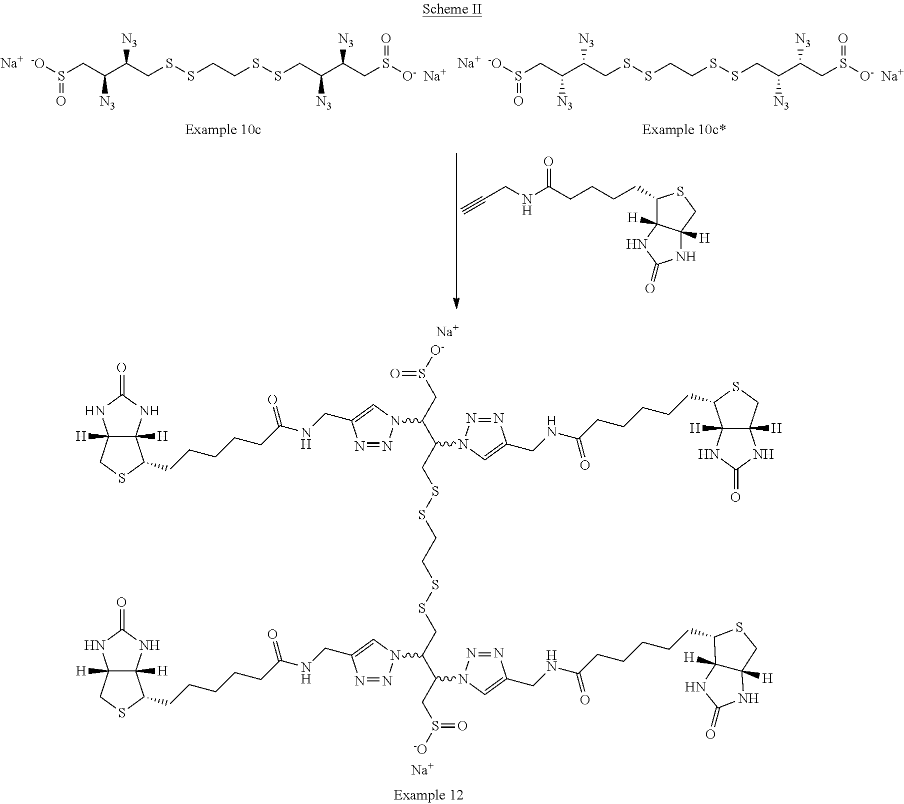

Scheme III depicts synthetic methodology to attach various dyes (e.g., fluorescein) to compounds of the invention employing thioisocyanate dyes (e.g., fluorescein-NCS) to afford the corresponding thiourea-linked compounds (e.g., Example 66).

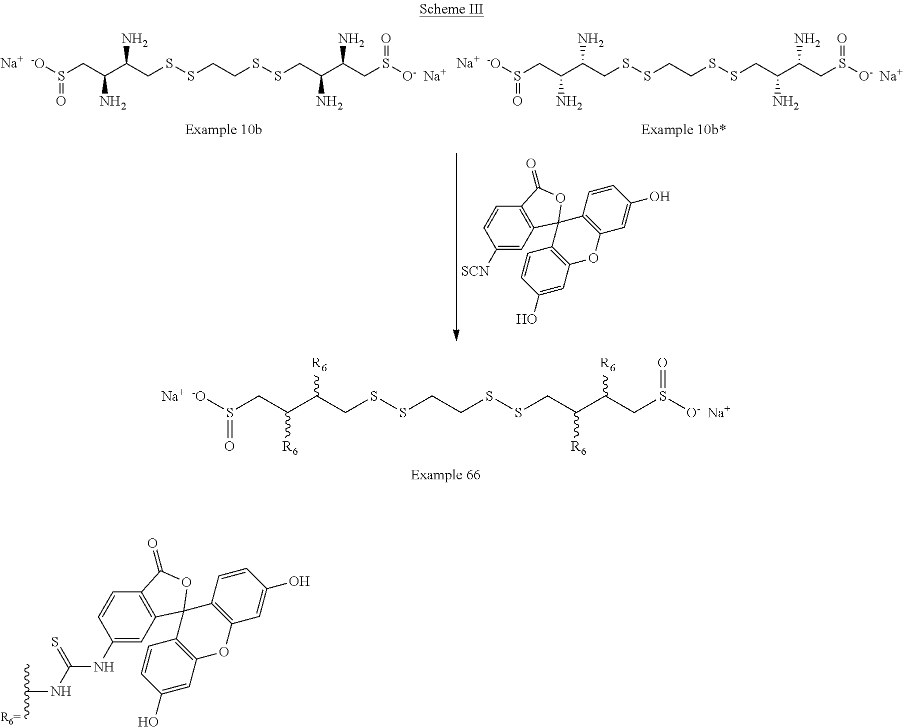

Alternatively, the isocyanate dye analogs (e.g., fluorescein-NCS) can be converted to the corresponding propoargyl-thiourea intermediates, which when reacted with azides 10c/10c*, affords Example 67 (Scheme IV).

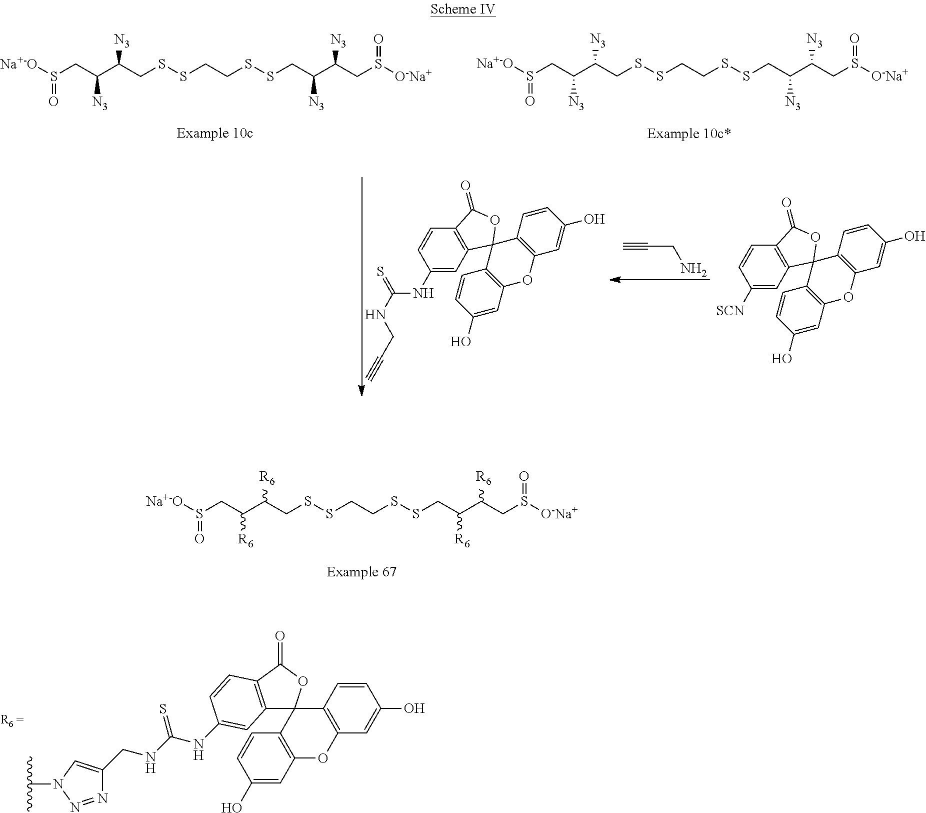

The following examples can be prepared according to one of Methods A, B, C, and/or D.

### Example 1

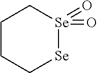

### 1,2-diselenane-1,1-dioxide (Method A)

### Example 2

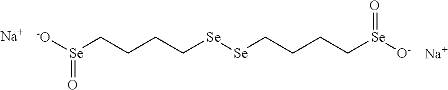

### sodium 4,4′-diselanediyldibutane-1-seleninate (Method B)

### Example 3

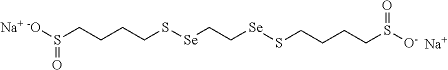

### sodium 5,10-dithia-6,9-diselenatetradecane-1,14-disulfinate (Method C)

### Example 4

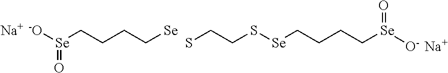

### sodium 6,9-dithia-5,10-diselenatetradecane-1,14-diseleninate (Method C)

### Example 5

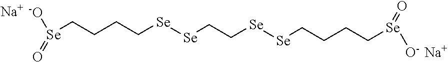

### sodium 4,4′-(ethane-1,2-diylbis(diselanediyl))dibutane-1-seleninate (Method C)

### Example 6

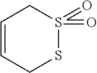

### 3,6-dihydro-1,2-dithiine-1,1-dioxide (Method A)

### Example (Z,Z)-7

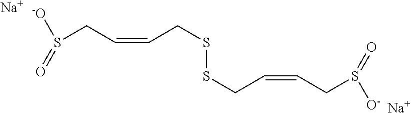

### sodium (2Z,2′Z)-4,4′-disulfanediyldibut-2-ene-1-sulfinate (Method B)

### Example (E,E)-7

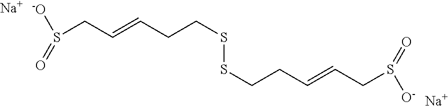

### sodium (2E,2′E)-5,5′-disulfanediyldipent-2-ene-1-sulfinate (Method B)

### Example 8a

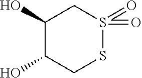

### trans-1,2-dithiane-4,5-diol-1,1-dioxide (Method A)

### Example 8b

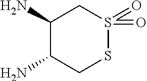

### trans-1,2-dithiane-4,5-diamino-1,1-dioxide (Method A)

### Example 8c

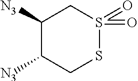 [SMILES: [N-]=[N+]=N[C@H]1CSS(=O)(=O)C[C@@H]1N=[N+]=[N-]]

### trans-1,2-dithiane-4,5-diazido-1,1-dioxide (Method A)

### Example 8d

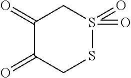

### 1,2-dithiane-4,5-dione-1,1-dioxide (Method A)

### Example 8e

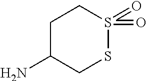

### 1,2-dithiane-4-amino-1,1-dioxide (Method A)

### Example 8f

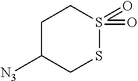 [SMILES: [N-]=[N+]=NC1CCS(=O)(=O)SC1]

### 1,2-dithiane-4-azido-1,1-dioxide (Method A)

### Example 8g

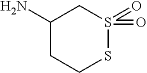

### 1,2-dithiane-5-amino-1,1-dioxide (Method A)

### Example 8h

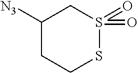 [SMILES: [N-]=[N+]=NC1CCSS(=O)(=O)C1]

### 1,2-dithiane-5-azido-1,1-dioxide (Method A)

### Example 8i

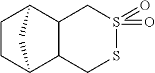 [SMILES: O=S1(=O)CC2C(CS1)[C@H]1CC[C@@H]2C1]

### Method A

### Example 9a

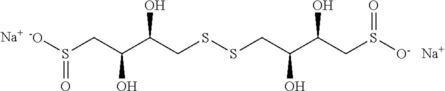

### sodium (2R,2′R,3R,3′R)-4,4′-disulfanediylbis(2,3-dihydroxybutane-1-sulfinate) (Method B)

### Example 9a*

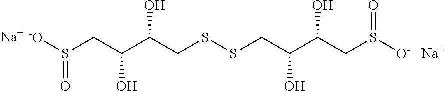

### sodium (2S,2′S,3S,3′S)-4,4′-disulfanediylbis(2,3-dihydroxybutane-1-sulfinate) (Method B)

### Example 9b

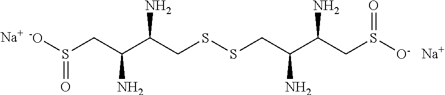

### sodium (2R,2′R,3R,3′R)-4,4′-disulfanediylbis(2,3-diaminobutane-1-sulfinate) (Method B)

### Example 9b*

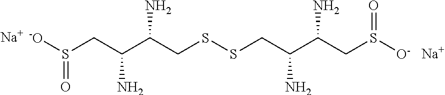

### sodium (2S,2′S,3S,3′S)-4,4′-disulfanediylbis(2,3-diaminobutane-1-sulfinate) (Method B)

### Example 9c

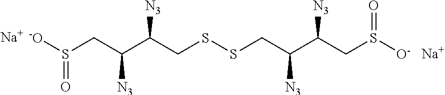

### sodium (2R,2′R,3R,3′R)-4,4′-disulfanediylbis(2,3-diazidobutane-1-sulfinate) (Method B)

### Example 9c*

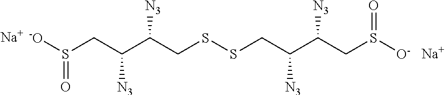

### sodium (2S,2′S,3S,3′S)-4,4′-disulfanediylbis(2,3-diazidobutane-1-sulfinate) (Method B)

### Example 9d

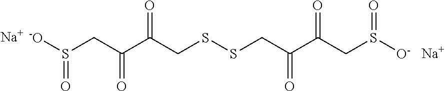

### sodium 4,4′-disulfanediylbis(2,3-dioxobutane-1-sulfinate) (Method B)

### Example 9e

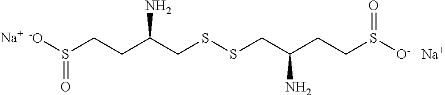

### sodium (3R,3′R)-4,4′-disulfanediylbis(3-aminobutane-1-sulfinate) (Method B)

### Example 9e*

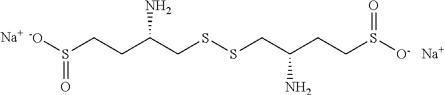

### sodium (3S,3′S)-4,4′-disulfanediylbis(3-aminobutane-1-sulfinate) (Method B)

### Example 9f

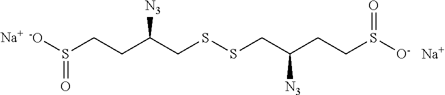

### sodium (3R,3′R)-4,4′-disulfanediylbis(3-azidobutane-1-sulfinate) (Method B)

### Example 9f*

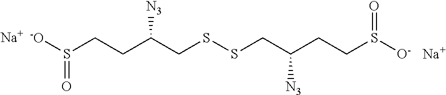

### sodium (3S,3′S)-4,4′-disulfanediylbis(3-azidobutane-1-sulfinate) (Method B)

### Example 9g

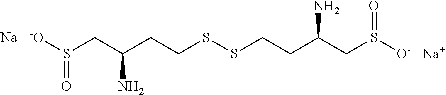

### sodium (2R,2′R)-4,4′-disulfanediylbis(2-aminobutane-1-sulfinate) (Method B)

### Example 9g*

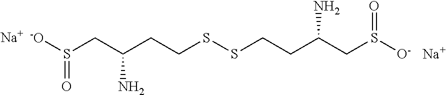

### sodium (2S,2′S)-4,4′-disulfanediylbis(2-aminobutane-1-sulfinate) (Method B)

### Example 9h

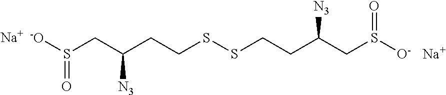

### sodium (2R,2′R)-4,4′-disulfanediylbis(2-azidobutane-1-sulfinate) (Method B)

### Example 9h*

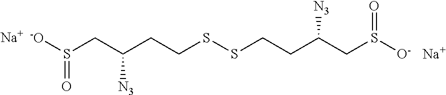

### sodium (2S,2′S)-4,4′-disulfanediylbis(2-azidobutane-1-sulfinate) (Method B)

### Example 9i

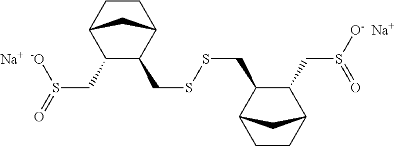

### sodium (1R,1′R,2R,2′R,3R,3′R,4S,4′S)-3,3′-disulfanediylbis(methylene)bis(bicyclo[2.2.1]heptane-3,2-diyl)dimethanesulfinate (Method B)

### Example 9i*

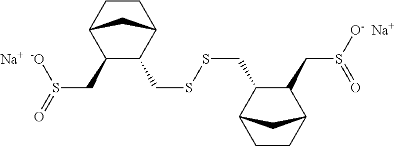

### sodium (1R,1′R,2S,2′S,3S,3′S,4S,4′S)-3,3′-disulfanediylbis(methylene)bis(bicyclo[2.2.1]heptane-3,2-diyl)dimethanesulfinate (Method B)

### Example 10a

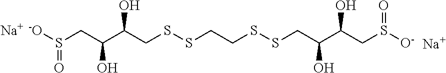

### sodium (2R,2′R,3R,3′R)-4,4′-(ethane-1,2-diylbis(disulfanediyl))bis(2,3-dihydroxybutane-1-sulfinate) (Method C)

### Example 10a*

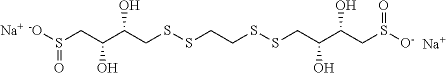

### sodium (2S,2′S,3S,3′S)-4,4′-(ethane-1,2-diylbis(disulfanediyl))bis(2,3-dihydroxybutane-1-sulfinate) (Method C)

### Example 10b

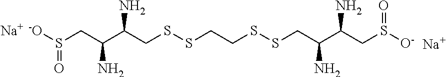

### sodium (2R,2′R,3R,3′R)-4,4′-(ethane-1,2-diylbis(disulfanediyl))bis(2,3-diaminobutane-1-sulfinate) (Method C)

### Example 10b*

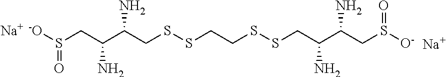

### (2S,2′S,3S,3′S)-4,4′-(ethane-1,2-diylbis(disulfanediyl))bis(2,3-diaminobutane-1-sulfinate) (Method C)

### Example 10c

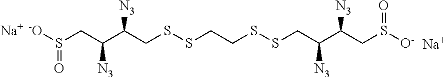

### (2R,2′R,3R,3′R)-4,4′-(ethane-1,2-diylbis(disulfanediyl))bis(2,3-diazidobutane-1-sulfinate) (Method C)

### Example 10c*

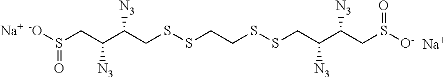

### sodium (2S,2′S,3S,3′S)-4,4′-(ethane-1,2-diylbis(disulfanediyl))bis(2,3-diazidobutane-1-sulfinate) (Method C)

### Example 10d

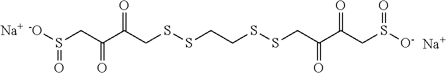

### sodium 4,4′-(ethane-1,2-diylbis(disulfanediyl))bis(2,3-dioxobutane-1-sulfinate) (Method C)

### Example 10e

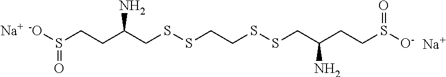

### sodium (3R,3′R)-4,4′-(ethane-1,2-diylbis(disulfanediyl))bis(3-aminobutane-1-sulfinate) (Method C)

### Example 10e*

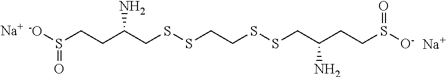

### sodium (3S,3′S)-4,4′-(ethane-1,2-diylbis(disulfanediyl))bis(3-aminobutane-1-sulfinate) (Method C)

### Example 10f

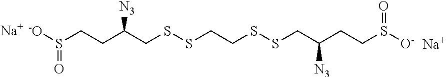

### sodium (3R,3′R)-4,4′-(ethane-1,2-diylbis(disulfanediyl))bis(3-azidobutane-1-sulfinate) (Method C)

### Example 10f*

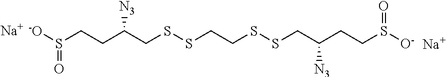

### sodium (3S,3′S)-4,4′-(ethane-1,2-diylbis(disulfanediyl))bis(3-azidobutane-1-sulfinate) (Method C)

### Example 10g

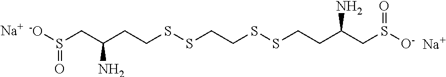

### sodium (2R,2′R)-4,4′-(ethane-1,2-diylbis(disulfanediyl))bis(2-aminobutane-1-sulfinate) (Method C)

### Example 10g*

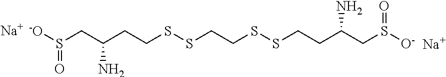

### sodium (2S,2′S)-4,4′-(ethane-1,2-diylbis(disulfanediyl))bis(2-aminobutane-1-sulfinate) (Method C)

### Example 10h

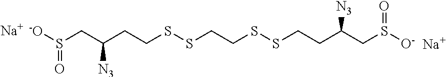

### sodium (2R,2′R)-4,4′-(ethane-1,2-diylbis(disulfanediyl))bis(2-azidobutane-1-sulfinate) (Method C)

### Example 10h*

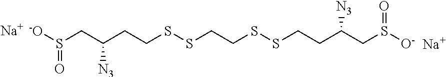

### sodium (2S,2′S)-4,4′-(ethane-1,2-diylbis(disulfanediyl))bis(2-azidobutane-1-sulfinate) (Method C)

### Example 10i

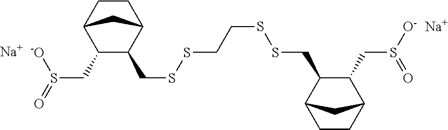

### sodium (1R,1′R,2R,2′R,3R,3′R,4S,4′S)-3,3′-(ethane-1,2-diylbis(disulfanediyl))bis(methylene)bis(bicyclo[2.2.1]heptane-3,2-diyl)dimethanesulfinate (Method C)

### Example 10i

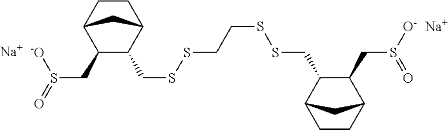

### sodium (1R,1′R,2S,2′S,3S,3′S,4S,4′S)-3,3′-(ethane-1,2-diylbis(disulfanediyl))bis(methylene)bis(bicyclo[2.2.1]heptane-3,2-diyl)dimethanesulfinate (Method C)

The following compounds of Formula III can be prepared in accordance with the general procedures presented herein.

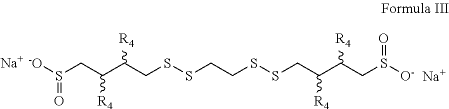

### Example 11

Compound of Formula III, wherein

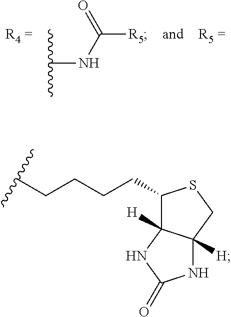

### Example 12

Compound of Formula III, wherein

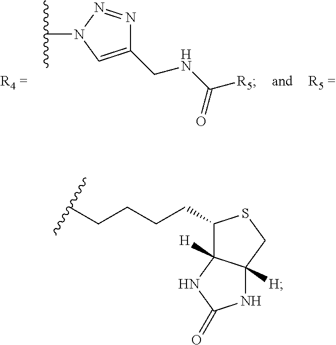

### Example 13

Compound of Formula III, wherein

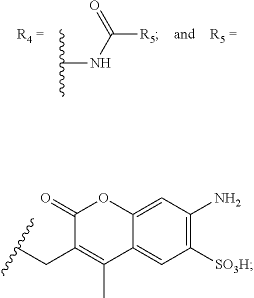

### Example 14

Compound of Formula III, wherein

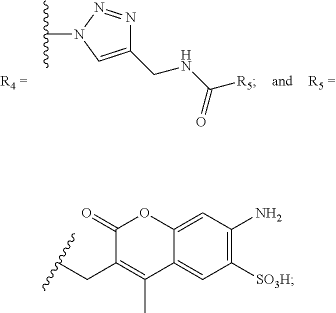

### Example 15

Compound of Formula III, wherein

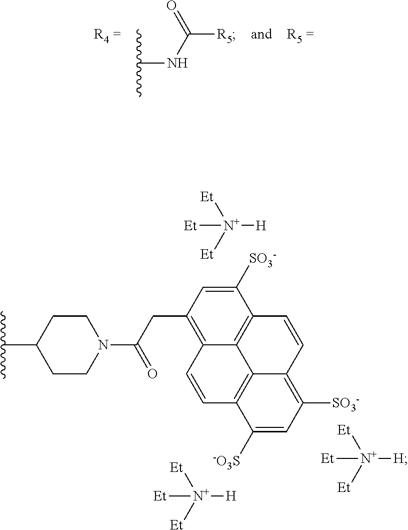

### Example 16

Compound of Formula III, wherein

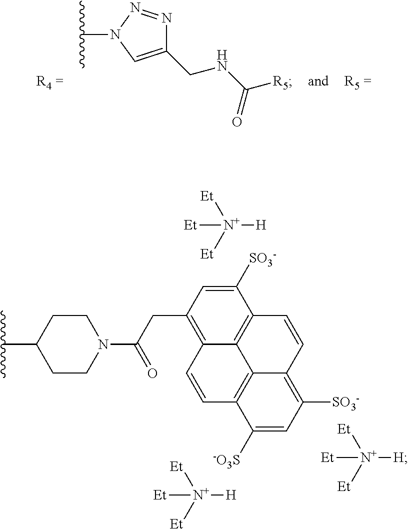

### Example 17

Compound of Formula III, wherein

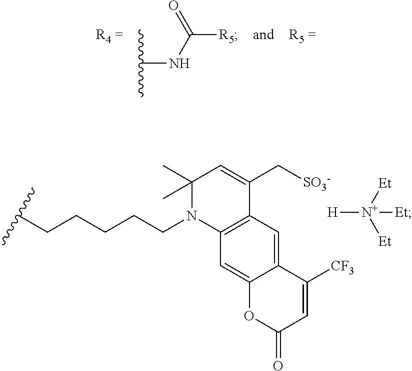

### Example 18

Compound of Formula III, wherein

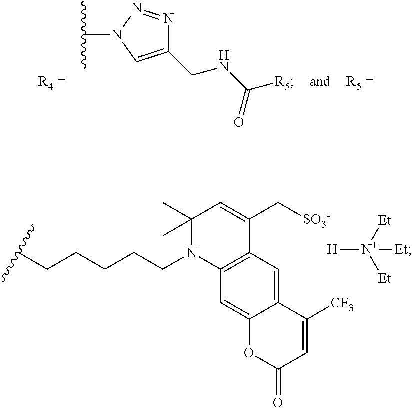

### Example 19

Compound of Formula III, wherein

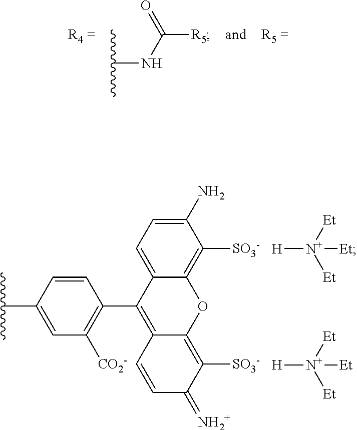

### Example 20

Compound of Formula III, wherein

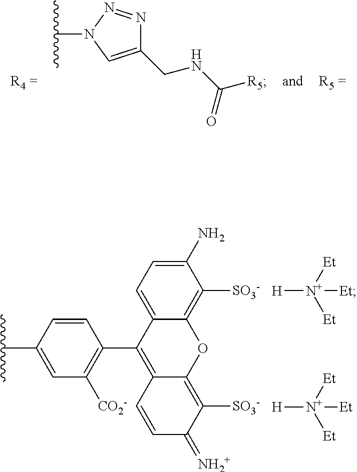

### Example 21

Compound of Formula III, wherein

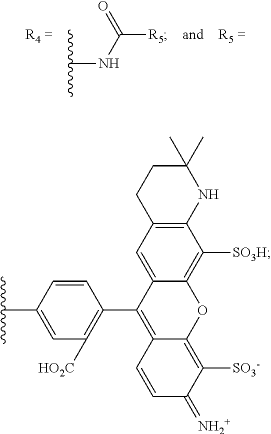

### Example 22

Compound of Formula III, wherein

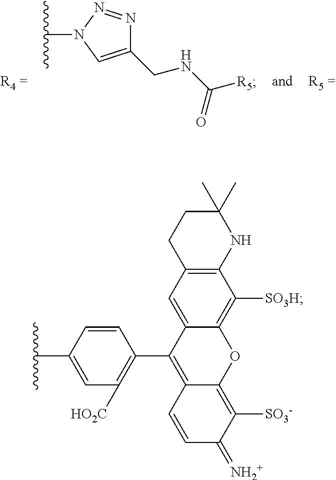

### Example 23

Compound of Formula III, wherein

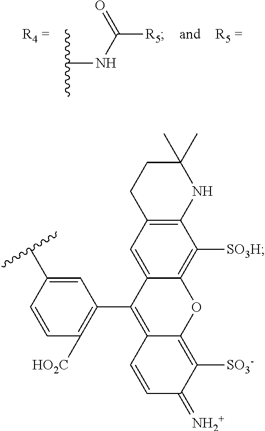

### Example 24

Compound of Formula III, wherein

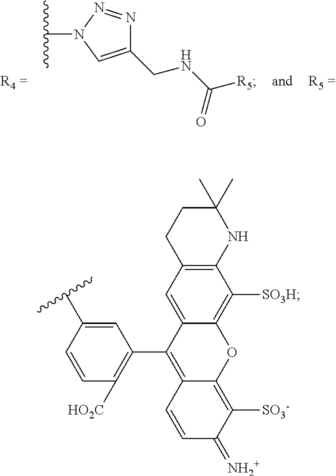

### Example 25

Compound of Formula III, wherein

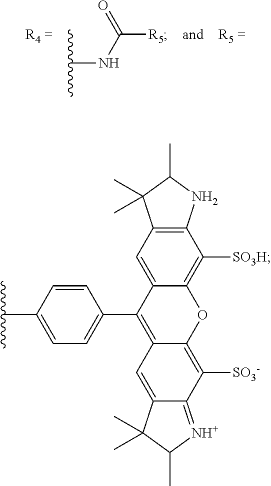

### Example 26

Compound of Formula III, wherein

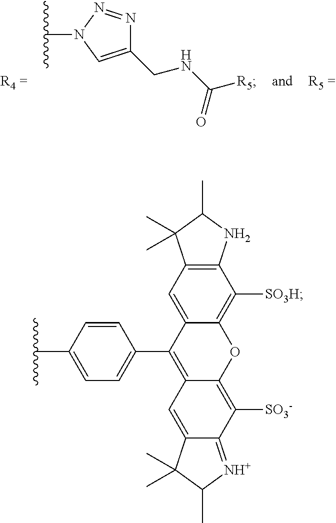

### Example 27

Compound of Formula III, wherein

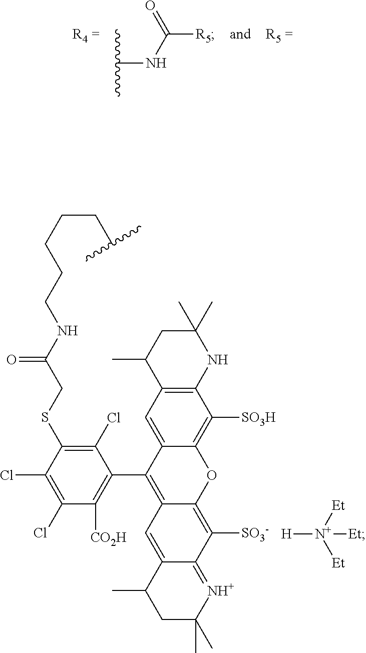

### Example 28

Compound of Formula III, wherein

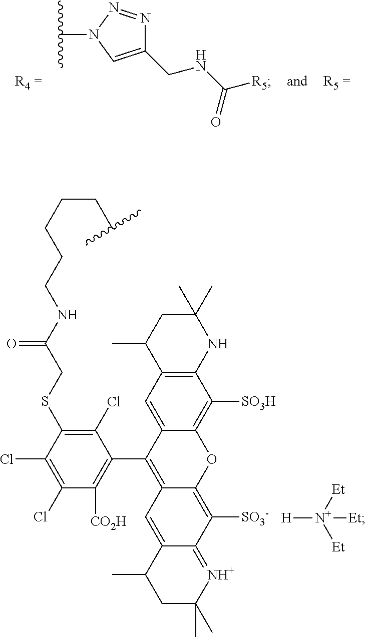

### Example 28

Compound of Formula III, wherein

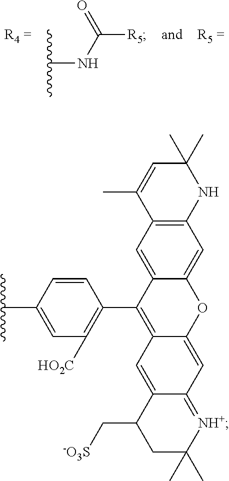

### Example 29

Compound of Formula III, wherein

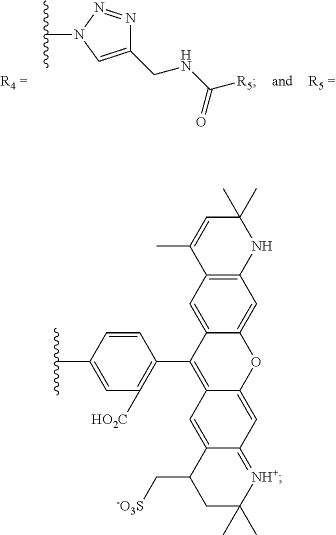

### Example 30

Compound of Formula III, wherein

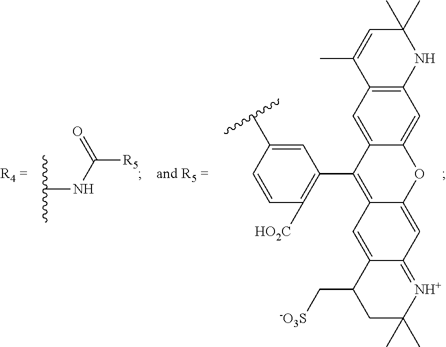

### Example 31

Compound of Formula III, wherein

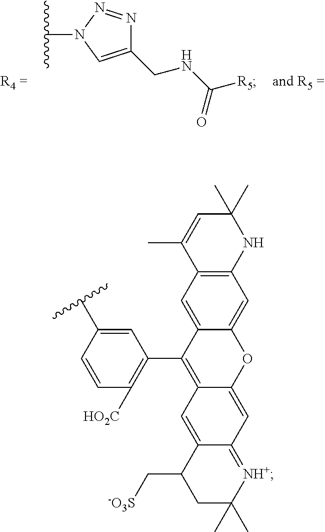

### Example 32

Compound of Formula III, wherein

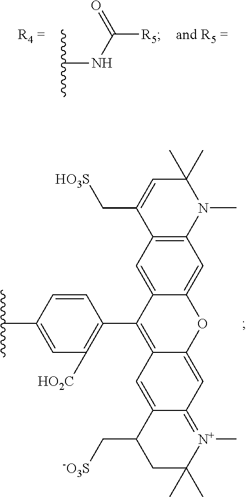

### Example 33

Compound of Formula III, wherein

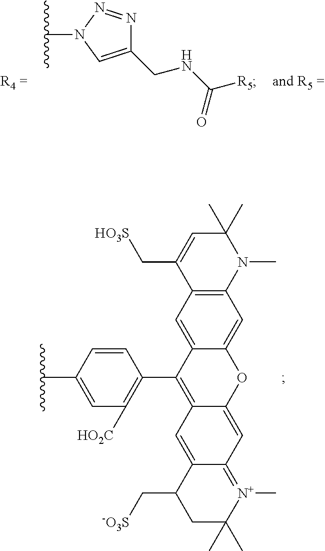

### Example 34

Compound of Formula III, wherein

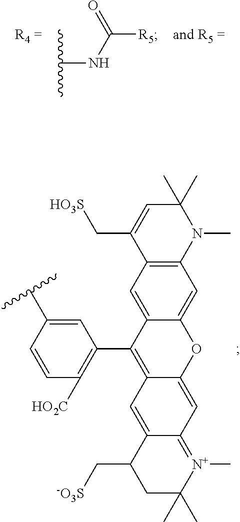

### Example 35

Compound of Formula III, wherein

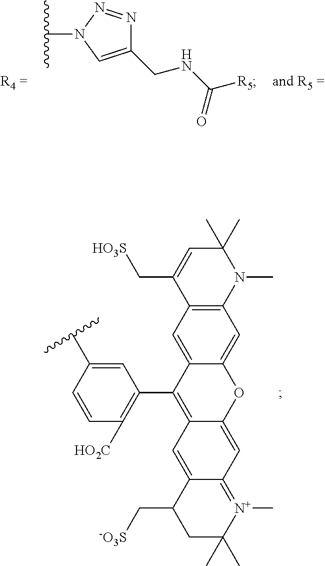

### Example 36

Compound of Formula III, wherein

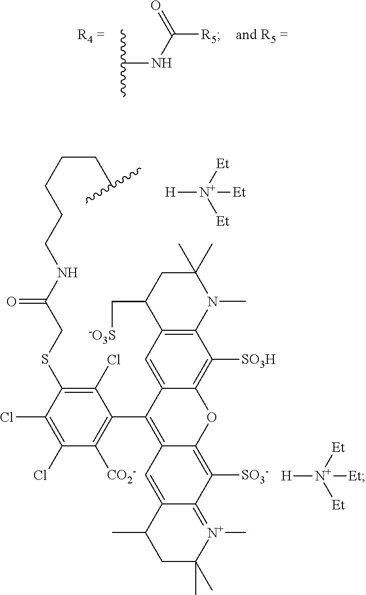

### Example 37

Compound of Formula III, wherein

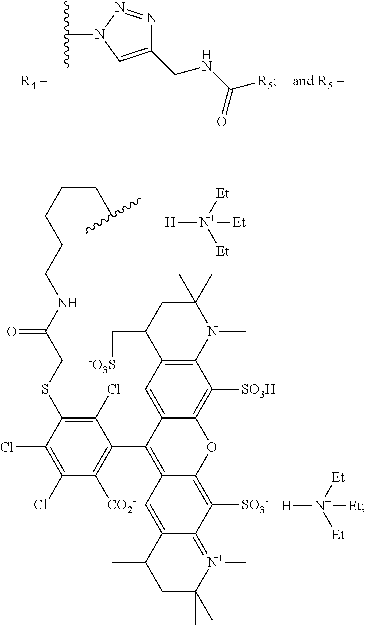

### Example 38

Compound of Formula III, wherein

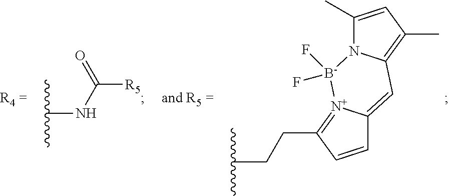

### Example 39

Compound of Formula III, wherein

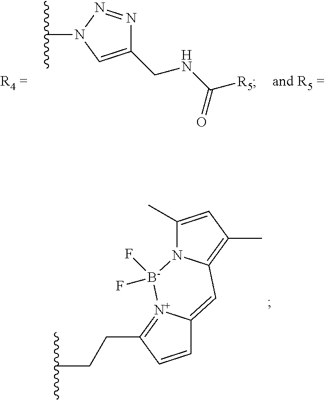

### Example 40

Compound of Formula III, wherein

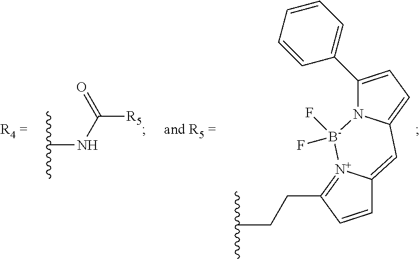

### Example 41

Compound of Formula III, wherein

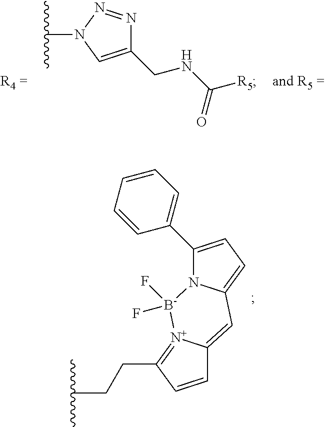

### Example 42

Compound of Formula III, wherein

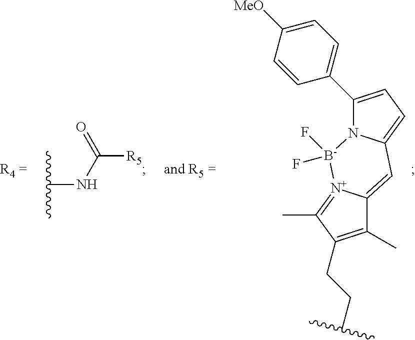

### Example 43

Compound of Formula III, wherein

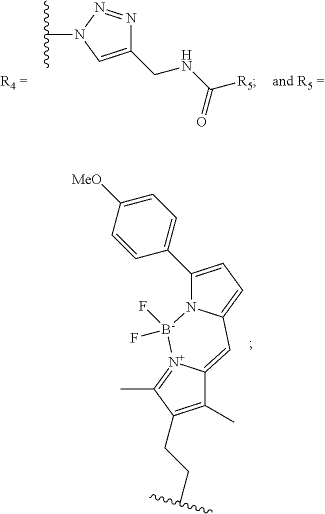

### Example 44

Compound of Formula III, wherein

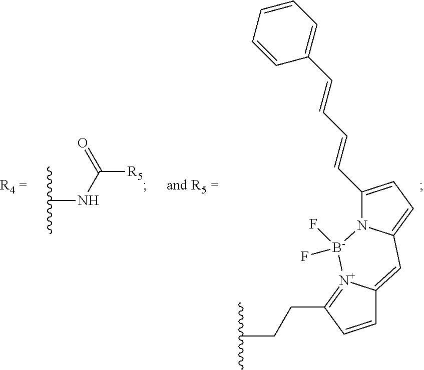

### Example 45

Compound of Formula III, wherein

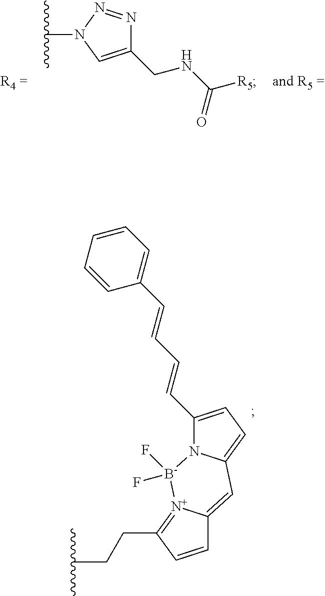

### Example 46

Compound of Formula III, wherein

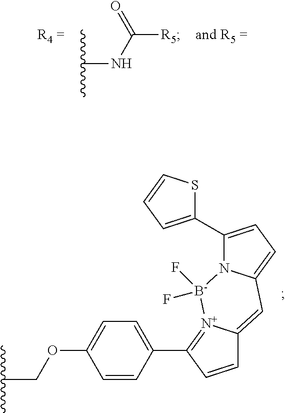

### Example 47

Compound of Formula III, wherein

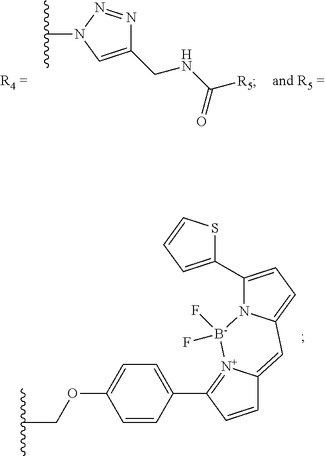

### Example 48

Compound of Formula III, wherein

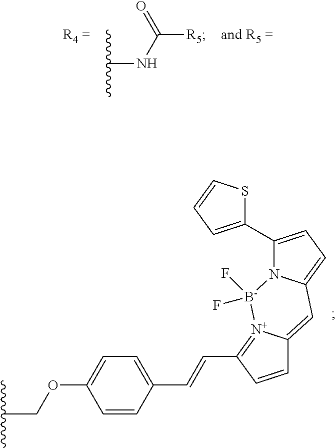

### Example 49

Compound of Formula III, wherein

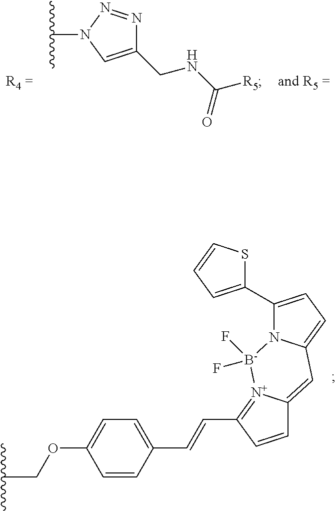

### Example 50

Compound of Formula III, wherein

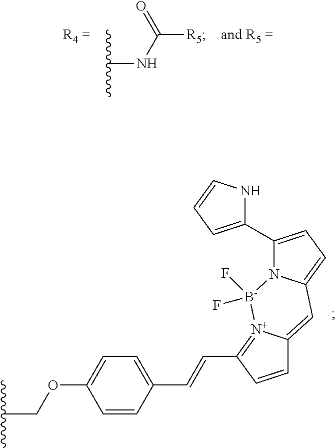

### Example 51

Compound of Formula III, wherein

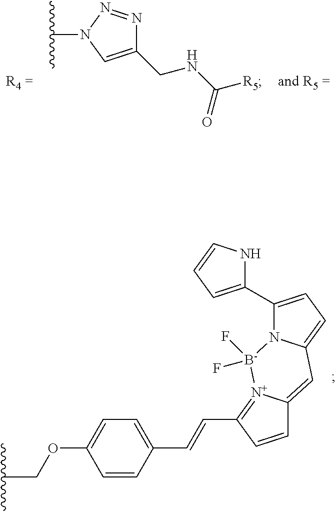

### Example 52

Compound of Formula III, wherein

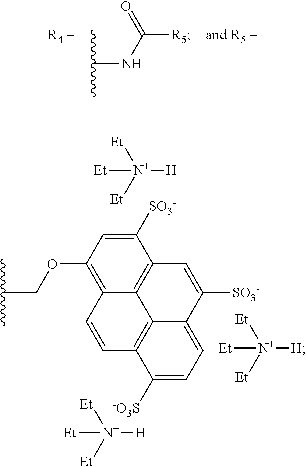

### Example 53

Compound of Formula III, wherein

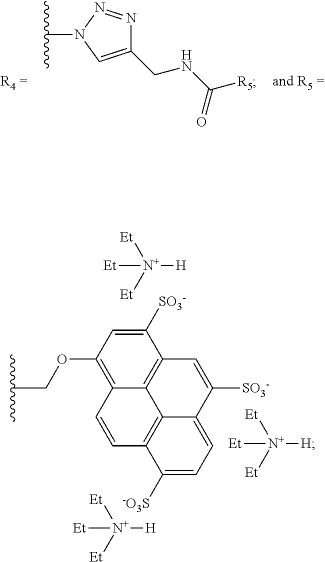

### Example 54

Compound of Formula III, wherein

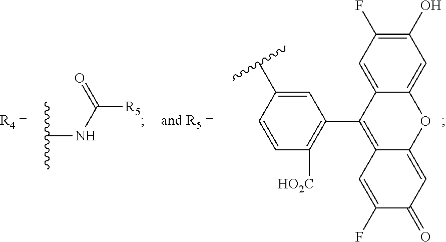

### Example 55

Compound of Formula III, wherein

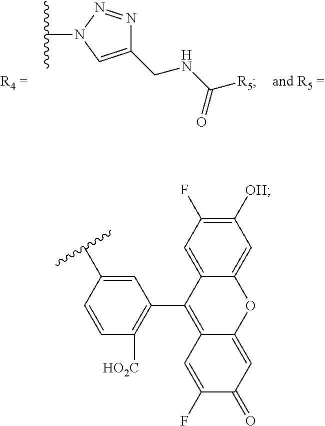

### Example 56

Compound of Formula III, wherein

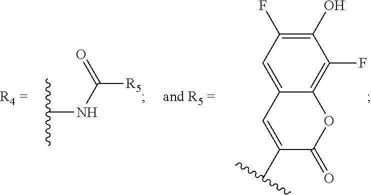

### Example 57

Compound of Formula III, wherein

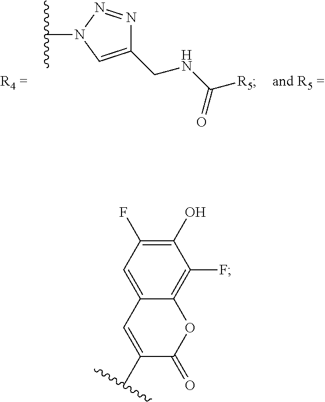

### Example 58

Compound of Formula III, wherein

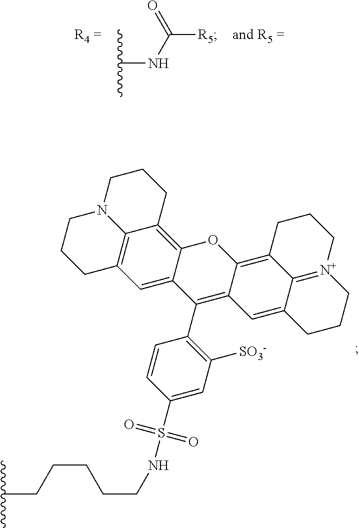

### Example 59

Compound of Formula III, wherein

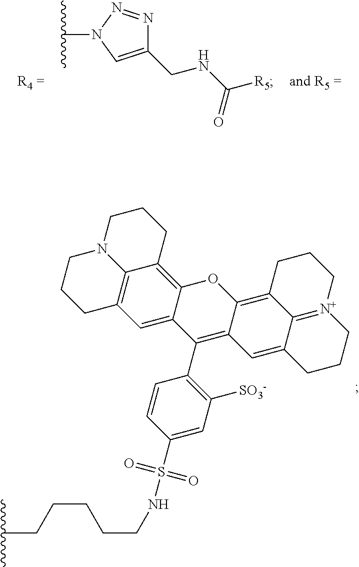

### Example 60

Compound of Formula III, wherein

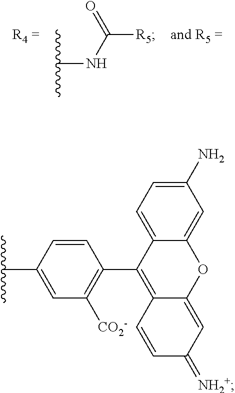

### Example 61

Compound of Formula III, wherein

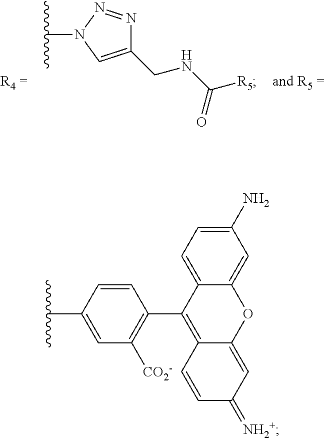

### Example 62

Compound of Formula III, wherein

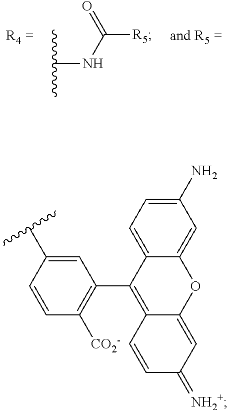

### Example 63

Compound of Formula III, wherein

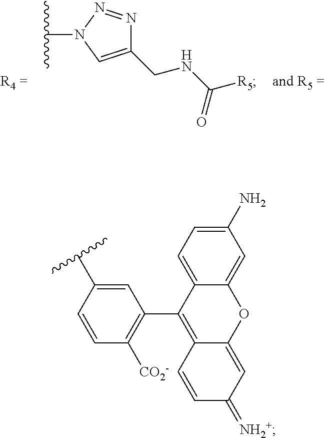

### Example 64

Compound of Formula III, wherein

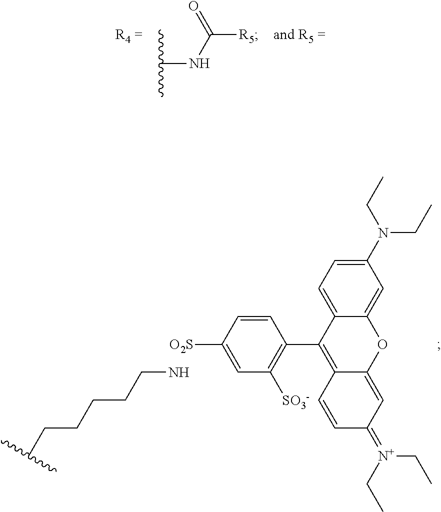

### Example 65

Compound of Formula III, wherein

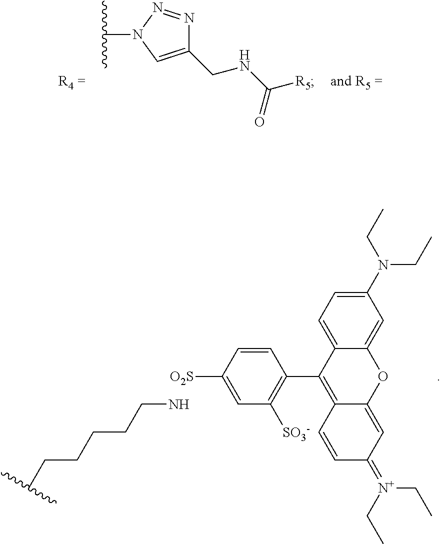

The following compounds of Formula IV can be prepared in accordance with the general procedures presented herein.

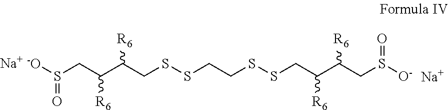

### Example 66

Compound of Formula IV, wherein

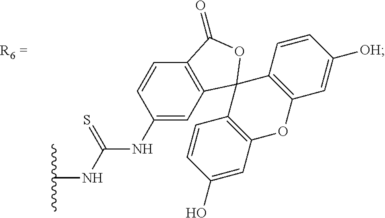 [SMILES: *NC(=S)Nc1ccc2c(c1)C1(OC2=O)c2ccc(O)cc2Oc2cc(O)ccc21]

### Example 67

Compound of Formula IV, wherein

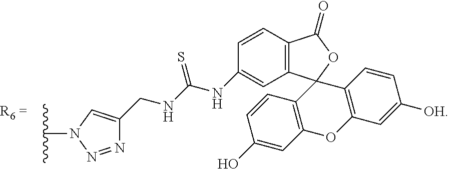 [SMILES: *n1cc(CNC(=S)Nc2ccc3c(c2)C2(OC3=O)c3ccc(O)cc3Oc3cc(O)ccc32)nn1]

### Example 68

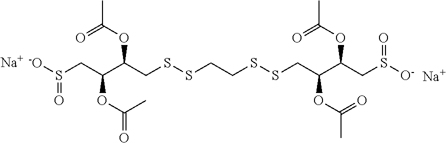

### sodium (2R,3R)-2,3-diacetoxy-4-((2-(((2R,3R)-2,3-diacetoxy-4-sulfinatobutyl)disulfanyl)ethyl)disulfanyl)butane-1-sulfinate

### Example 69

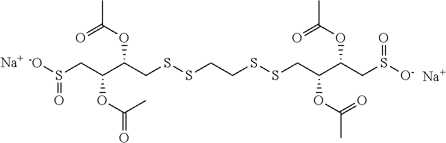

### sodium (2S,3S)-2,3-diacetoxy-4-((2-(((2S,3S)-2,3-diacetoxy-4-sulfinatobutyl)disulfanyl)ethyl)disulfanyl)butane-1-sulfinate

### Example 70

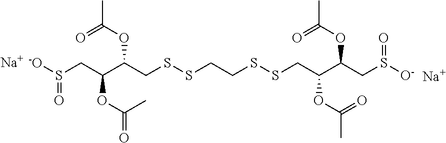

### sodium (2S,3R)-2,3-diacetoxy-4-((2-(((2R,3S)-2,3-diacetoxy-4-sulfinatobutyl)disulfanyl)ethyl)disulfanyl)butane-1-sulfinate

### Example 71

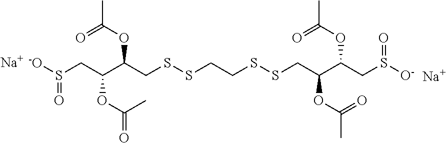

### sodium (2R,3S)-2,3-diacetoxy-4-((2-(((2S,3R)-2,3-diacetoxy-4-sulfinatobutyl)disulfanyl)ethyl)disulfanyl)butane-1-sulfinate

### Example 72

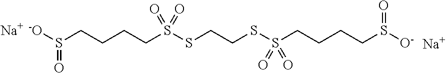

### sodium 4-(2-(4-sulfinatobutylsulfonylthio)ethylthiosulfonyl)butane-1-sulfinate

### Example 73

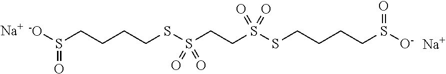

### sodium 4-(2-(4-sulfinatobutylthiosulfonyl)ethylsulfonylthio)butane-1-sulfinate

### Example 74

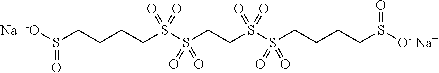

### sodium 4-(2-(4-sulfinatobutylsulfonylsulfonyl)ethylsulfonylsulfonyl)butane-1-sulfinate

### Example 75

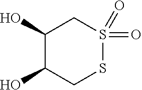

### cis-1,2-dithiane-4,5-diol-1,1-dioxide

### Example 76

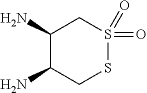

### cis-1,2-dithiane-4,5-diamino-1,1-dioxide

### Example 77

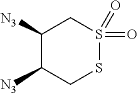 [SMILES: [N-]=[N+]=N[C@H]1CS(=O)(=O)SC[C@H]1N=[N+]=[N-]]

### cis-1,2-dithiane-4,5-diazido-1,1-dioxide

### Example 78

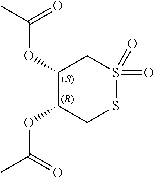

### 1,2-dithiane-(4R,5S-diacetoxy)-1,1-dioxide

### Example 79

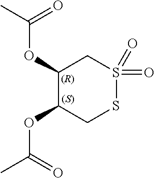

### 1,2-dithiane-(4S,5R-diacetoxy)-1,1-dioxide

### Example 80

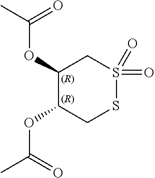

### 1,2-dithiane-(4R,5R-diacetoxy)-1,1-dioxide

### Example 81

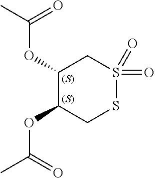

### 1,2-dithiane-(4S,5S-diacetoxy)-1,1-dioxide

### Example 82

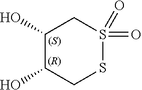

### 1,2-dithiane-(4R,5S-dihydroxy)-1,1-dioxide

### Example 83

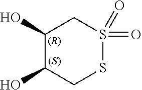

### 1,2-dithiane-(4S,5R-dihydroxy)-1,1-dioxide

### Example 84

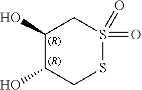

### 1,2-dithiane-(4R,5R-dihydroxy)-1,1-dioxide

### Example 85

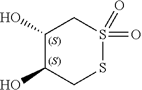

### 1,2-dithiane-(4S,5S-dihydroxy)-1,1-dioxide

### Example 86

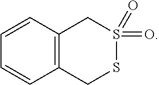 [SMILES: O=S1(=O)Cc2ccccc2CS1]

### Example 87

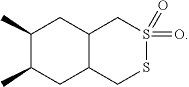

### Example 88

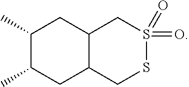

### Example 89

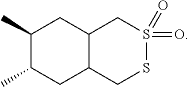

### Example 90

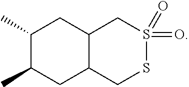

### Example 91

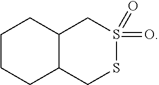 [SMILES: O=S1(=O)CC2CCCCC2CS1]

A summary of the compounds tested and their activity against cancer cells is presented in FIG. 5C. RBF3 produced a 50% decrease in the viability of MDA-MB-468 cells and the HER2 overexpressing BT474 cells between 5-10 μM (FIG. 5D). RBF3's effects on cell proliferation were observed as low as 2 μM; a concentration at which RBF3 had no effect on the proliferation of immortalized human mammary epithelial cells (FIG. 5E).

Examination of the biochemical effects of RBF3 on MDA-MB-468 (FIG. 5F), SKBR3 (FIG. 5G), and BT474 (FIG. 5H) cells revealed that RBF3 decreased the levels of EGFR, HER2, and HER3 in parallel. RBF3 was more effective than NSC624203 at downregulating EGFR and upregulating PARP cleavage in side-by-side comparisons in MDA-MB-468 cells. In contrast to the high activity of RBF1 and RBF3, derivatives of these compounds in which the sulfinate groups had been oxidized to sulfonate groups, RBF4 and RBF6, did not downregulate EGFR, HER2, or HER3 in MDA-MB-468 cells, did not increase PARP cleavage, and did not reduce Akt phosphorylation (FIG. 5F). These observations indicate that the sulfonate groups present in these compounds are one factor affecting their activity against cancer cells. One possible interpretation of these data is that oxidation of the sulfinate groups to sulfonate causes loss of activity because, unlike the sulfinate sulfur, the sulfonate sulfur does not behave as a nucleophile and, therefore, cannot disrupt the extracellular disulfide bonds in EGFR, HER2, and HER3 and destabilize these proteins.

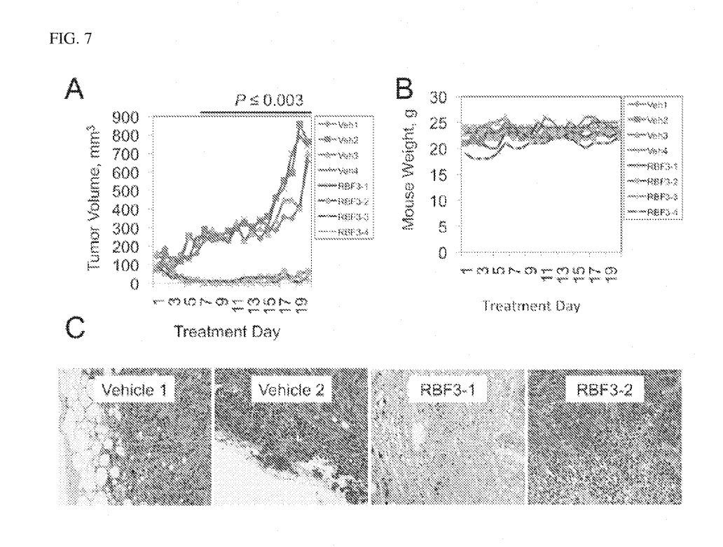

Given the promising impact of RBF3 on the viability of HER2 and EGFR overexpressing breast cancer cell lines, we examined whether RBF3 had activity against xenografts of human breast cancer. Strikingly, 40 mg/kg RBF3 strongly suppressed the growth of tumors derived from BT474 cells (FIG. 7A). In contrast, vehicle (water) treated tumors grew rapidly. During the treatment period the weights of the animals were not significantly affected by drug treatment (FIG. 7B). Examination of the histology of the remnants of RBF3 treated tumors revealed that most of the tumor tissue was necrotic or fibrotic, and that only a small fraction of these tumors was composed of viable cancer cells (FIG. 7C). In separate experiments, we treated tumor-bearing mice with RBF3 at dosages of up to 160 mg/kg/day. Under these conditions, no evidence of toxicity was observed based on histological examination of kidney, liver, lung, and brain tissue (FIG. 7D). In contrast, tumor tissues from RBF3-treated animals exhibited a high frequency of cancer cell death.

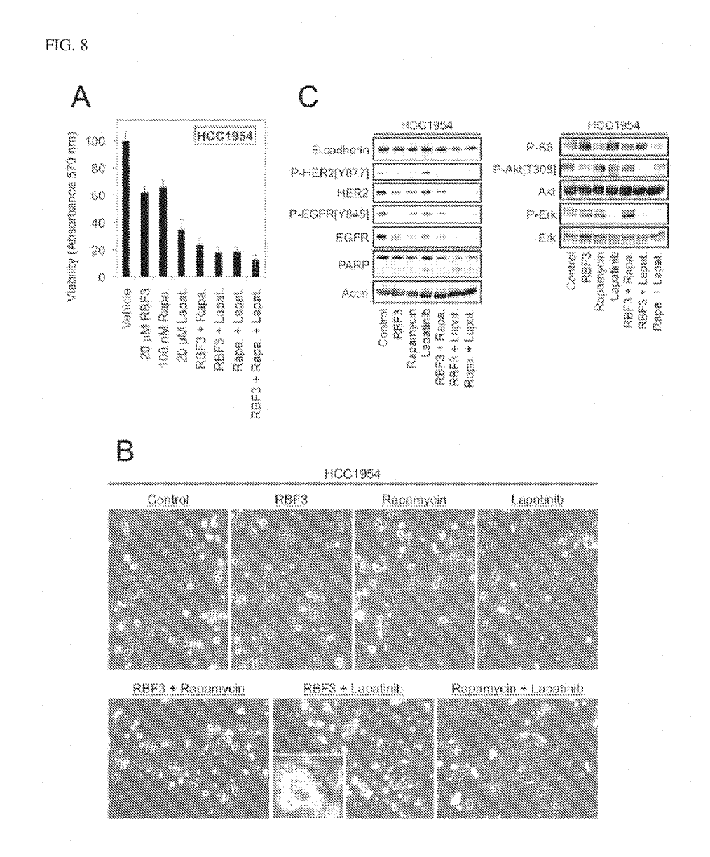

A major problem in the clinical management of HER2-positive breast cancer is the acquisition of resistance to HER2-targeted drugs such as Trastuzumab and Lapatinib. One possible mechanism responsible for Trastuzumab resistance is the acquisition of constitutive signaling through the phosphatidylinositol 3′-kinase (PI3K) pathway caused either by activating PI3K point mutations to produce excessive PIP3 or through the mutational inactivation of the PIP3 phosphatase PTEN. The HCC1954 breast cancer cell line is resistant to Trastuzumab due to a H1047R mutation in PIK3CA [Weigelt, B., Warne, P. H., and Downward, J. (2011) PIK3CA mutation, but not PTEN loss of function, determines the sensitivity of breast cancer cells to mTOR inhibitory drugs Oncogene 30, 3222-3233]. Cell viability assays indicated that HCC1954 cells were relatively resistant to RBF3 and rapamycin treatment, and slightly more responsive to Lapatinib treatment (FIG. 8A). However, pairwise combination of two of these three drugs was more effective at decreasing cell viability than any of the drugs alone. In particular, the combination of RBF3 and Lapatinib resulted in massive HCC1954 cell death (FIG. 8B) Immunoblot analyses indicated that RBF3 decreased HER2 and EGFR expression, but not E-cadherin levels (FIG. 8C). Combined treatment with RBF3 and Lapatinib decreased HER2 and EGFR to undetectable levels. As expected, rapamycin treatment suppressed S6 phosphorylation, but did not alter Akt or Erk phosphorylation. RBF3 decreased Akt phosphorylation, but did not alter Erk phosphorylation, while Lapatinib reduced Erk phosphorylation without affecting Akt phosphorylation. Combined RBF3 and Lapatinib treatment reduced Akt phosphorylation to a greater extent than RBF3 alone and decreased Erk phosphorylation to the same extent as Lapatinib alone. Of the three binary drug combinations, RBF3+Lapatinib induced the largest fractional increase in the cleavage of PARP.

### DISCUSSION

Conventional drugs that act on HER2, EGFR, and HER3 are either monoclonal antibodies or tyrosine kinase inhibitors. DDAs represent a new way of inactivating these oncogenes by downregulating them at the protein level.

The conserved disufide bonding pattern in the extracellular domains of EGFR family members provides an additional approach for targeting these oncogenes. The observation in FIG. 8 that RBF3 abrogates Akt phosphorylation in parallel with HER2/EGFR/HER3 downregulation, while Lapatinib treatment of the same cells blocks Erk phosphorylation without affecting Akt phosphorylation suggests that the differences between the mechanisms of RBF3 and Lapatinib action will produce additive or synergistic anti-cancer effects when paired in combination therapies. This is supported by the observation that RBF3+Lapatinib more effectively reduce the viability of Trastuzumab resistant HCC1954 cells more than either drug alone. This cooperative effect correlates with a greater extent of EGFR and HER2 downregulation, a higher fractional PARP cleavage, and more complete Akt dephosphorylation on Thr³⁰⁸.

Thiol-reactive groups have also found use in the synthesis of irreversible kinase inhibitors targeting the ATP binding pocket [Bridges, A. J. (1999) The rationale and strategy used to develop a series of highly potent, irreversible, inhibitors of the epidermal growth factor receptor family of tyrosine kinases Curr Med Chem 6, 825-843; Fry, D. W., Bridges, A. J., Denny, W. A., Doherty, A., Greis, K. D., Hicks, J. L., Hook, K. E., Keller, P. R., Leopold, W. R., Loo, J. A., McNamara, D. J., Nelson, J. M., Sherwood, V., Smaill, J. B., Trumpp-Kallmeyer, S., and Dobrusin, E. M. (1998) Specific, irreversible inactivation of the epidermal growth factor receptor and erbB2, by a new class of tyrosine kinase inhibitor Proc Natl Acad Sci USA 95, 12022-12027; Singh, J., Dobrusin, E. M., Fry, D. W., Haske, T., Whitty, A., and McNamara, D. J. (1997) Structure-based design of a potent, selective, and irreversible inhibitor of the catalytic domain of the erbB receptor subfamily of protein tyrosine kinases J Med Chem 40, 1130-1135; Leproult, E., Barluenga, S., Moras, D., Wurtz, J. M., and Winssinger, N. (2011) Cysteine mapping in conformationally distinct kinase nucleotide binding sites: application to the design of selective covalent inhibitors J Med Chem 54, 1347-13551. In these instances a thiol-reactive group is appended to an ATP competitive inhibitor in such a way that the thiol-reactive group forms a covalent linkage with the side chain of a Cysteine residue. The advantage of this approach is that it can dramatically improve the selectivity of the resulting kinase inhibitors to only those kinases that harbor a Cysteine residue in the necessary location. Similarly, it may be possible to utilize DDAs as a disulfide bond-reactive moiety that can be appended to another ligand with protein-specific docking capability in order to specifically destabilize the targeted protein.

In summary, DDAs show impressive anticancer activity in mice without obvious toxicity. This class of agents is useful in the treatment of HER2- and EGFR-dependent breast tumors and may be effective for the treatment of cancers that have acquired resistance to monoclonal antibodies or tyrosine kinase inhibitors targeting these enzymes.

1. A compound of formula I, or a salt thereof represented by: 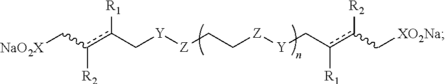
wherein, each X is independently S or Se;
each Y is independently S, SO₂, or Se;
each Z is independently S, SO₂, or Se;
each R₁ is independently selected from H, NH₂, N₃, OH, oxo, NH—R₃, OAc, 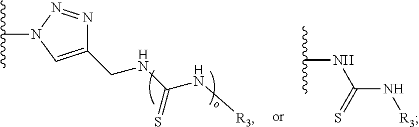
each R₂ is independently selected from H, NH₂, N₃, OH, oxo, NH—R₃, OAc, 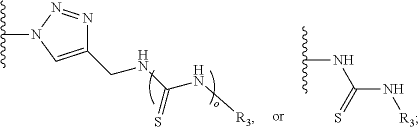
each R₃ is independently selected from biotin, fluorescein, AlexaFluor® dyes, BODIPY®, Cascade Blue®, coumarins, Oregon Green®, Pacific Blue™, Pacific Green™, Pacific Orange™ Rhodamine Green™, Rhodamine Red™, or Texas Red®;
or adjacent R₁, R₂ moieties, and the carbon atoms to which they are attached form an optionally substituted cycloalkyl moiety;
each n is independently 0 or 1;
each o is independently 0 or 1; and [symbol]
denotes a carbon-carbon single bond or double bond;
wherein if every X, Y, and Z is simultaneously S, then at least one of R₁ and R₂ is NH₂, N₃, OH, oxo, NH—R₃, OAc,

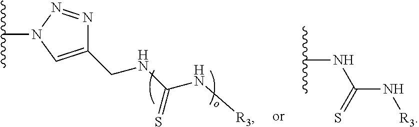

2. A compound of formula II, or a salt thereof: 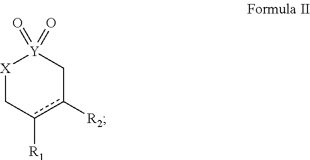
wherein, X is S or Se;
Y is S or Se;
R₁ is selected from H, NH₂, N₃, OAc, alkyl, or OH;
R₂ is selected from H, NH₂, N₃, OAc, alkyl, or OH;
or R₁, R₂, and the carbon atoms to which they are attached for an optionally substituted cycloalkyl moiety or an optionally substituted aryl moiety; and [symbol]
denotes a carbon-carbon single bond or double bond;
wherein if X and Y are both simultaneously S, then at least one of R₁ and R₂ is NH₂, N₃, OAc, alkyl, or OH.

3. A method of treating a subject suffering from or susceptible to a cell proliferative disorder comprising administering to the subject in need thereof a therapeutically effective amount of the compound of the following formula, or a salt thereof: 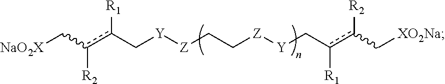
wherein, each X is independently S or Se;
each Y is independently S, SO₂, or Se;
each Z is independently S, SO₂, or Se;
each R₁ is independently selected from H, NH₂, N₃, OH, oxo, NH—R₃, OAc, 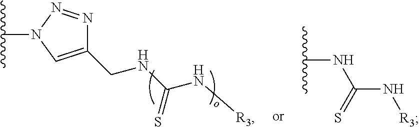
each R₂ is independently selected from H, NH₂, N₃, OH, oxo, NH—R₃, OAc, 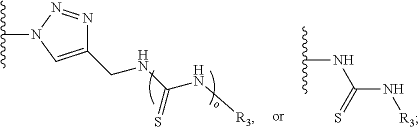
each R₃ is independently selected from biotin, fluorescein, AlexaFluor® dyes, BODIPY®, Cascade Blue®, coumarins, Oregon Green®, Pacific Blue™, Pacific Green™, Pacific Orange™ Rhodamine Green™, Rhodamine Red™, or Texas Red®;
or adjacent R₁, R₂ moieties, and the carbon atoms to which they are attached form an optionally substituted cycloalkyl moiety;
each n is independently 0 or 1;
each o is independently 0 or 1; and [symbol]
denotes a carbon-carbon single bond or double bond.

4. The method of claim 3, wherein the cell proliferative disorder is cancer.

5. The method of claim 4, wherein the cancer is HER2 mediated.

6. The method of claim 4, wherein the cancer is breast cancer.

7. The method of claim 6, wherein the breast cancer is HER2-positive breast cancer.

8. The method of claim 6, wherein the breast cancer is mediated by HER2, HER3, and/or EGFR.

9. The method of claim 8, wherein the compound inhibits at least one of HER2, HER3, or EGFR.

10. The method of claim 8, wherein the compound inhibits at least two of HER2, HER3, or EGFR.

11. The method of claim 8, wherein the compound inhibits HER2, HER3, and EGFR.

12.-23. (canceled)

24. The compound of claim 1, wherein the compound is:
sodium (2R,3R)-2,3-diacetoxy-4-((2-(((2R,3R)-2,3-diacetoxy-4-sulfinatobutyl)disulfanyl)ethyl)disulfanyl)butane-1-sulfinate;
sodium (2S,3S)-2,3-diacetoxy-4-((2-(((2S,3S)-2,3-diacetoxy-4-sulfinatobutyl)disulfanyl)ethyl)disulfanyl)butane-1-sulfinate;
sodium (2S,3R)-2,3-diacetoxy-4-((2-(((2R,3S)-2,3-diacetoxy-4-sulfinatobutyl)disulfanyl)ethyl)disulfanyl)butane-1-sulfinate;
sodium (2R,3S)-2,3-diacetoxy-4-((2-(((2S,3R)-2,3-diacetoxy-4-sulfinatobutyl)disulfanyl)ethyl)disulfanyl)butane-1-sulfinate;
sodium 4-(2-(4-sulfinatobutylsulfonylthio)ethylthiosulfonyl)butane-1-sulfinate;
sodium 4-(2-(4-sulfinatobutylthiosulfonyl)ethylsulfonylthio)butane-1-sulfinate
sodium 4-(2-(4-sulfinatobutylsulfonylsulfonyl)ethylsulfonylsulfonyl)butane-1-sulfinate;
sodium 4,4′-diselanediyldibutane-1-seleninate;
sodium 5,10-dithia-6,9-diselenatetradecane-1,14-disulfinate;
sodium 6,9-dithia-5,10-diselenatetradecane-1,14-diseleninate;
sodium 4,4′-(ethane-1,2-diylbis(diselanediyl))dibutane-1-seleninate;
sodium (2Z,2′Z)-4,4′-disulfanediyldibut-2-ene-1-sulfinate;
sodium (2E,2′E)-5,5′-disulfanediyldipent-2-ene-1-sulfinate;
sodium (2R,2′R,3R,3′R)-4,4′-disulfanediylbis(2,3-dihydroxybutane-1-sulfinate);
sodium (2S,2′S,3S,3′S)-4,4′-disulfanediylbis(2,3-dihydroxybutane-1-sulfinate);
sodium (2R,2′R,3R,3′R)-4,4′-disulfanediylbis(2,3-diaminobutane-1-sulfinate);
sodium (2S,2′S,3S,3′S)-4,4′-disulfanediylbis(2,3-diaminobutane-1-sulfinate);
sodium (2R,2′R,3R,3′R)-4,4′-disulfanediylbis(2,3-diazidobutane-1-sulfinate);
sodium (2S,2′S,3S,3′S)-4,4′-disulfanediylbis(2,3-diazidobutane-1-sulfinate);
sodium 4,4′-disulfanediylbis(2,3-dioxobutane-1-sulfinate);
sodium (3R,3′R)-4,4′-disulfanediylbis(3-aminobutane-1-sulfinate);
sodium (3S,3′S)-4,4′-disulfanediylbis(3-aminobutane-1-sulfinate);
sodium (3R,3′R)-4,4′-disulfanediylbis(3-azidobutane-1-sulfinate);
sodium (3S,3′S)-4,4′-disulfanediylbis(3-azidobutane-1-sulfinate);
sodium (2R,2′R)-4,4′-disulfanediylbis(2-aminobutane-1-sulfinate);
sodium (2S,2′S)-4,4′-disulfanediylbis(2-aminobutane-1-sulfinate);
sodium (2R,2′R)-4,4′-disulfanediylbis(2-azidobutane-1-sulfinate);
sodium (2S,2′S)-4,4′-disulfanediylbis(2-azidobutane-1-sulfinate);
sodium (1R,1′R,2R,2′R,3R,3′R,4S,4′S)-3,3′-disulfanediylbis(methylene)bis(bicyclo[2.2.1]heptane-3,2-diyl)dimethanesulfinate;
sodium (1R,1′R,2S,2′S,3S,3′S,4S,4′S)-3,3′-disulfanediylbis(methylene)bis(bicyclo[2.2.1]heptane-3,2-diyl)dimethanesulfinate;
sodium (2R,2′R,3R,3′R)-4,4′-(ethane-1,2-diylbis(disulfanediyl))bis(2,3-dihydroxybutane-1-sulfinate);
sodium (2S,2′S,3S,3′S)-4,4′-(ethane-1,2-diylbis(disulfanediyl))bis(2,3-dihydroxybutane-1-sulfinate);
sodium (2R,2′R,3R,3′R)-4,4′-(ethane-1,2-diylbis(disulfanediyl))bis(2,3-diaminobutane-1-sulfinate);
(2S,2′S,3S,3′S)-4,4′-(ethane-1,2-diylbis(disulfanediyl))bis(2,3-diaminobutane-1-sulfinate);
(2R,2′R,3R,3′R)-4,4′-(ethane-1,2-diylbis(disulfanediyl))bis(2,3-diazidobutane-1-sulfinate);
sodium (2S,2′S,3S,3′S)-4,4′-(ethane-1,2-diylbis(disulfanediyl))bis(2,3-diazidobutane-1-sulfinate);
sodium 4,4′-(ethane-1,2-diylbis(disulfanediyl))bis(2,3-dioxobutane-1-sulfinate);
sodium (3R,3′R)-4,4′-(ethane-1,2-diylbis(disulfanediyl))bis(3-aminobutane-1-sulfinate);
sodium (3S,3′S)-4,4′-(ethane-1,2-diylbis(disulfanediyl))bis(3-aminobutane-1-sulfinate);
sodium (3R,3′R)-4,4′-(ethane-1,2-diylbis(disulfanediyl))bis(3-azidobutane-1-sulfinate);
sodium (3S,3′S)-4,4′-(ethane-1,2-diylbis(disulfanediyl))bis(3-azidobutane-1-sulfinate);
sodium (2R,2′R)-4,4′-(ethane-1,2-diylbis(disulfanediyl))bis(2-aminobutane-1-sulfinate);
sodium (2S,2′S)-4,4′-(ethane-1,2-diylbis(disulfanediyl))bis(2-aminobutane-1-sulfinate);
sodium (2R,2′R)-4,4′-(ethane-1,2-diylbis(disulfanediyl))bis(2-azidobutane-1-sulfinate);
sodium (2S,2′S)-4,4′-(ethane-1,2-diylbis(disulfanediyl))bis(2-azidobutane-1-sulfinate);
sodium (1R,1′R,2R,2′R,3R,3′R,4S,4′S)-3,3′-(ethane-1,2-diylbis(disulfanediyl))bis(methylene)bis(bicyclo[2.2.1]heptane-3,2-diyl)dimethanesulfinate; or
sodium (1R,1′R,2S,2′S,3S,3′S,4S,4′S)-3,3′-(ethane-1,2-diylbis(disulfanediyl))bis(methylene)bis(bicyclo[2.2.1]heptane-3,2-diyl)dimethanesulfinate.

25. The compound of claim 2, wherein the compound is:
1,2-diselenane-1,1-dioxide;
3,6-dihydro-1,2-dithiine-1,1-dioxide;
trans-1,2-dithiane-4,5-diol-1,1-dioxide;
trans-1,2-dithiane-4,5-diamino-1,1-dioxide;
trans-1,2-dithiane-4,5-diazido-1,1-dioxide;
cis-1,2-dithiane-4,5-diol-1,1-dioxide;
cis-1,2-dithiane-4,5-diamino-1,1-dioxide;
cis-1,2-dithiane-4,5-diazido-1,1-dioxide;
1,2-dithiane-4,5-dione-1,1-dioxide;
1,2-dithiane-(4R,5S-diacetoxy)-1,1-dioxide;
1,2-dithiane-(4S,5R-diacetoxy)-1,1-dioxide;
1,2-dithiane-(4R,5R-diacetoxy)-1,1-dioxide;
1,2-dithiane-(4S,5S-diacetoxy)-1,1-dioxide;
1,2-dithiane-(4R,5S-dihydroxy)-1,1-dioxide;
1,2-dithiane-(4S,5R-dihydroxy)-1,1-dioxide;
1,2-dithiane-(4R,5R-dihydroxy)-1,1-dioxide;
1,2-dithiane-(4S,5S-dihydroxy)-1,1-dioxide;
1,2-dithiane-4-amino-1,1-dioxide;
1,2-dithiane-4-azido-1,1-dioxide;
1,2-dithiane-5-amino-1,1-dioxide;
1,2-dithiane-5-azido-1,1-dioxide;

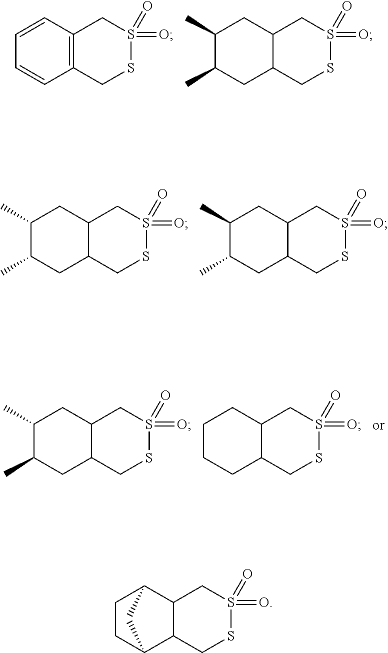

26. The compound of claim 1, according to Formula V: 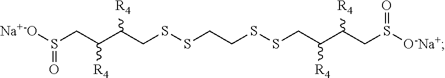
wherein, each R₄ is independently selected from 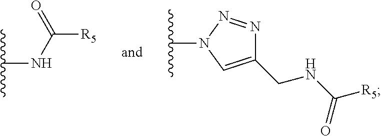
and
each R₅ is independently selected from the group consisting of:

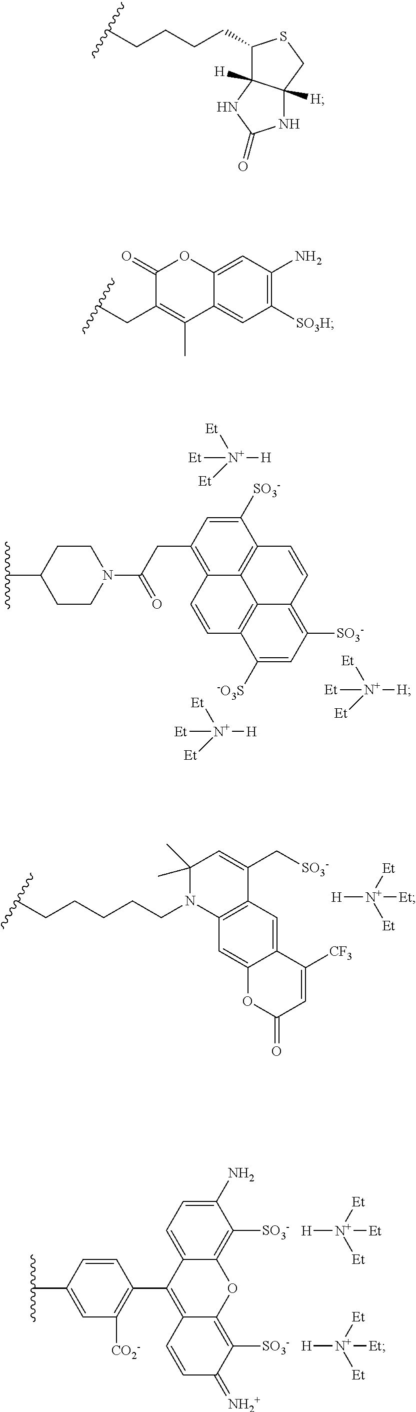

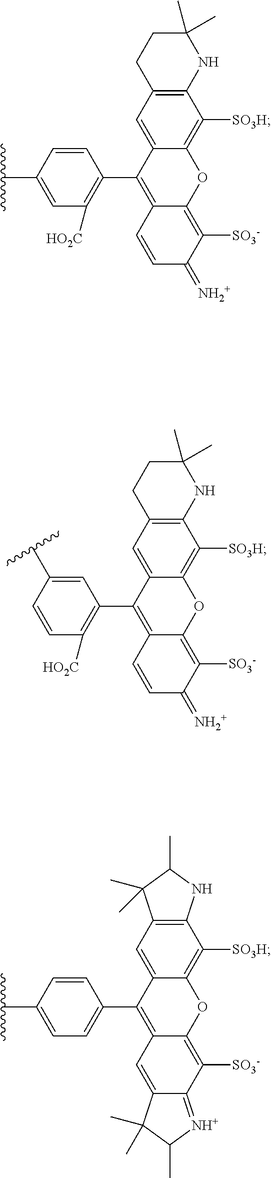

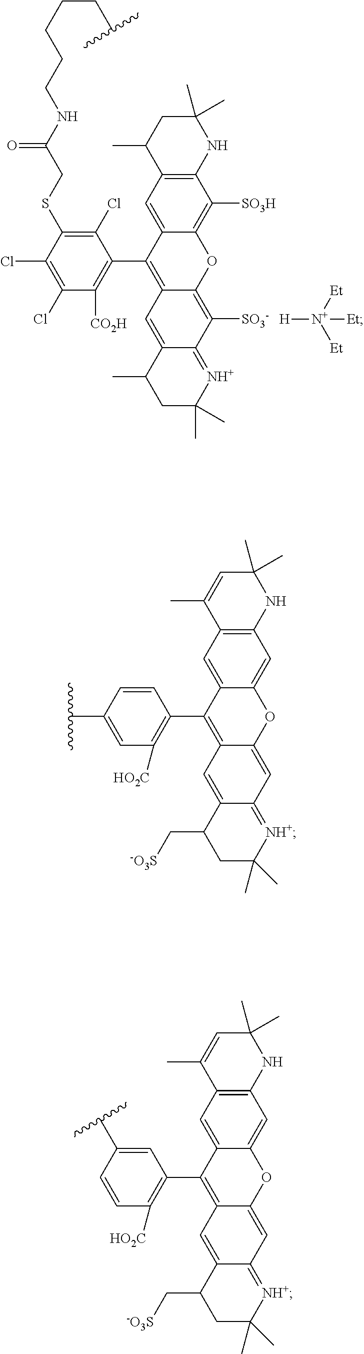

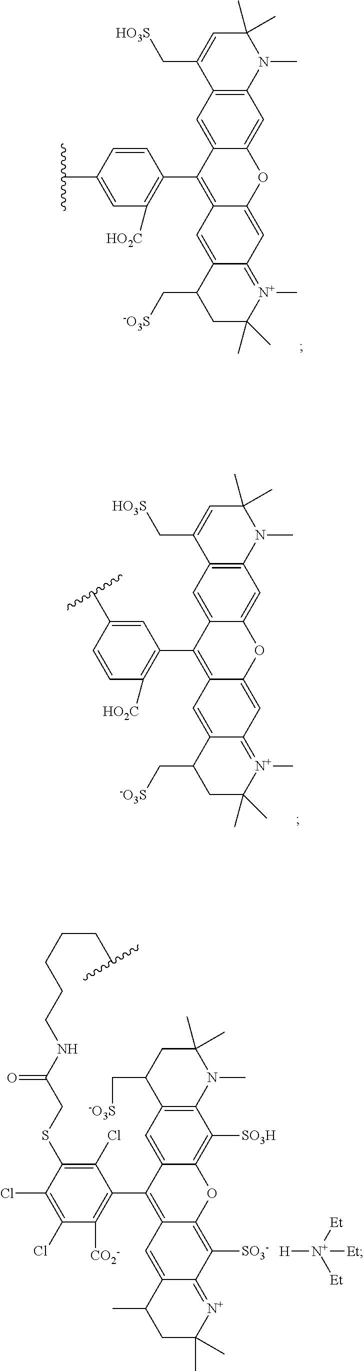

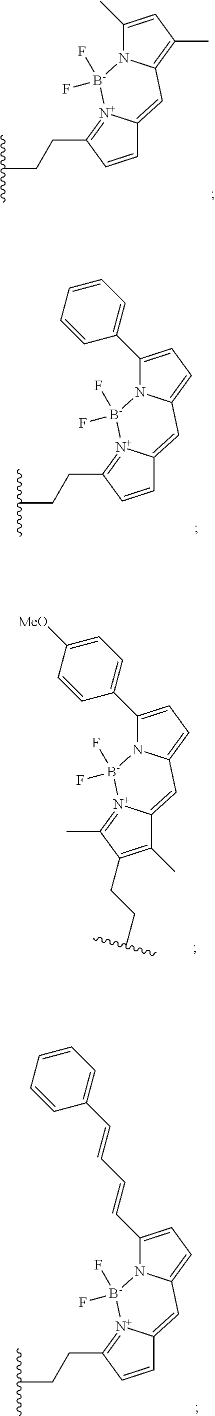 [SMILES: *CCC1=[N+]2C(=Cc3c(C)cc(C)n3[B-]2(F)F)C=C1 ; *CCC1=[N+]2C(=Cc3ccc(-c4ccccc4)n3[B-]2(F)F)C=C1 ; *CCC1=C(C)C2=Cc3ccc(-c4ccc(OC)cc4)n3[B-](F)(F)[N+]2=C1C ; *CCC1=[N+]2C(=Cc3ccc(/C=C/C=C/c4ccccc4)n3[B-]2(F)F)C=C1]

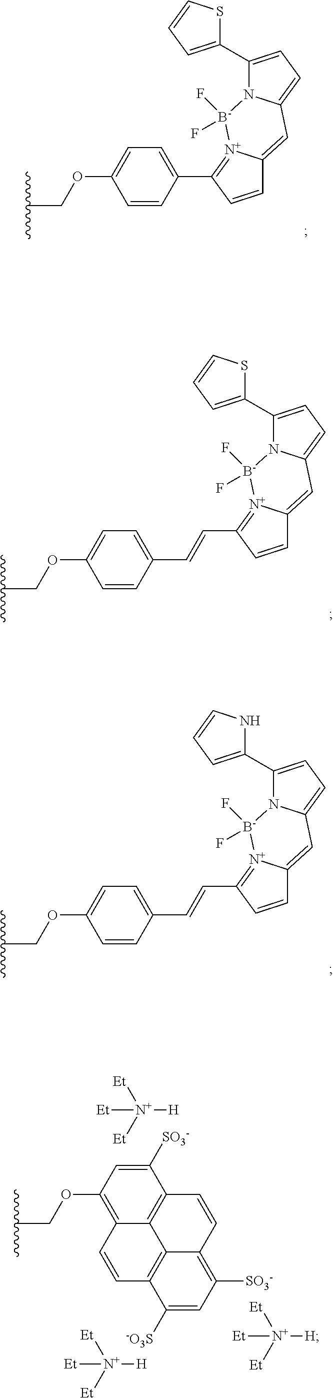

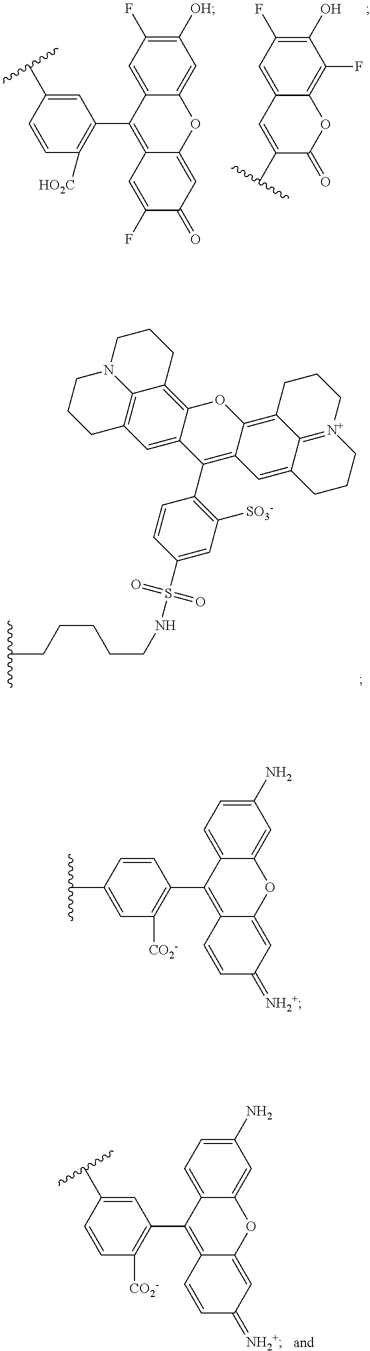

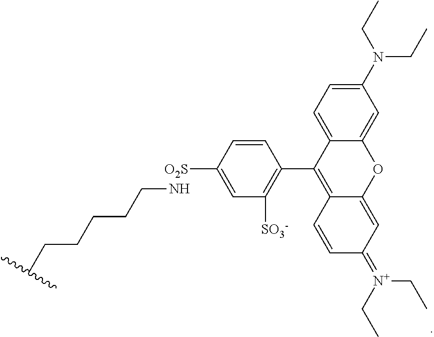

27. The compound of claim 26, wherein the compound is selected from the group consisting of:

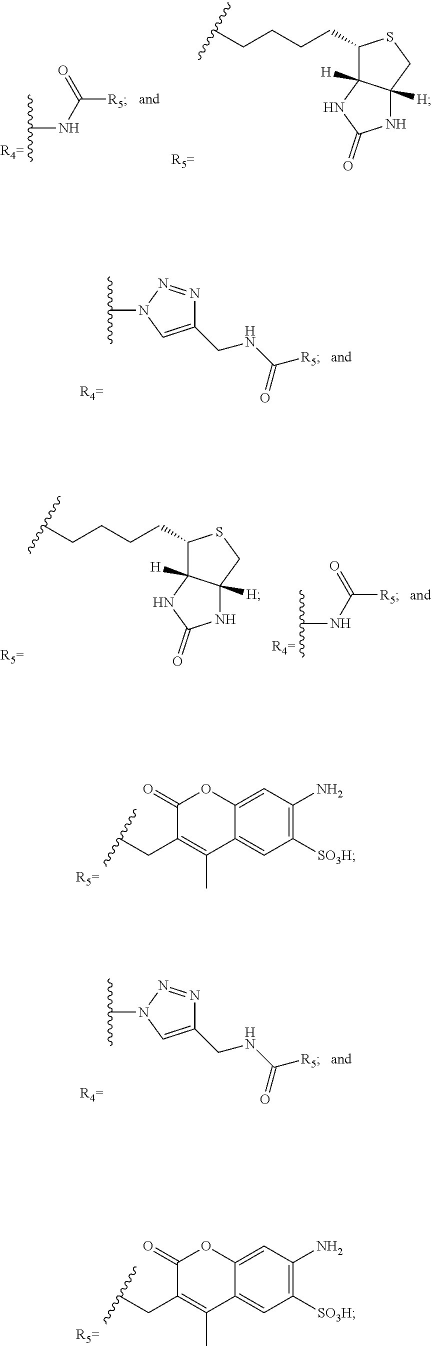

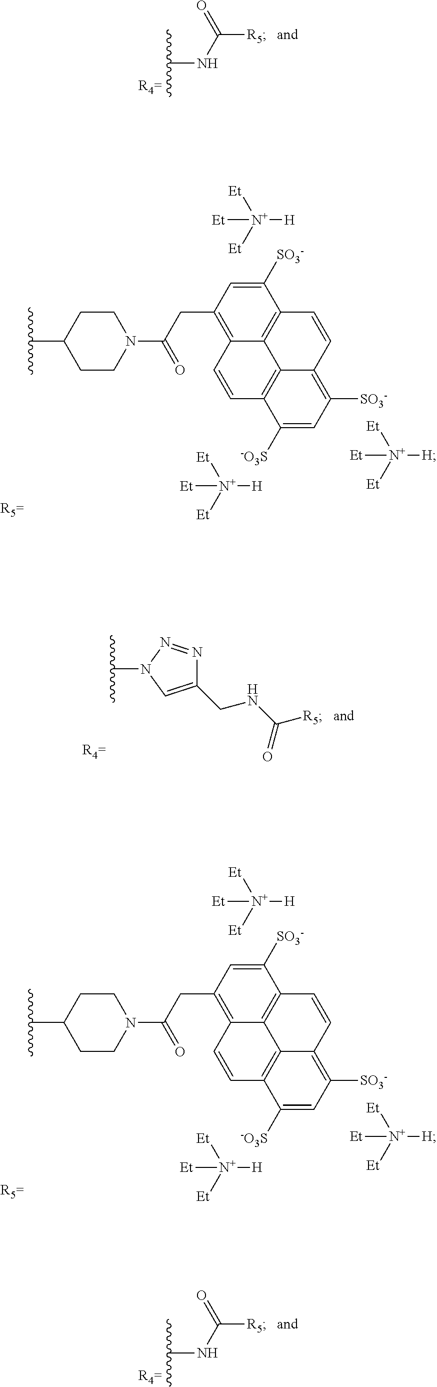

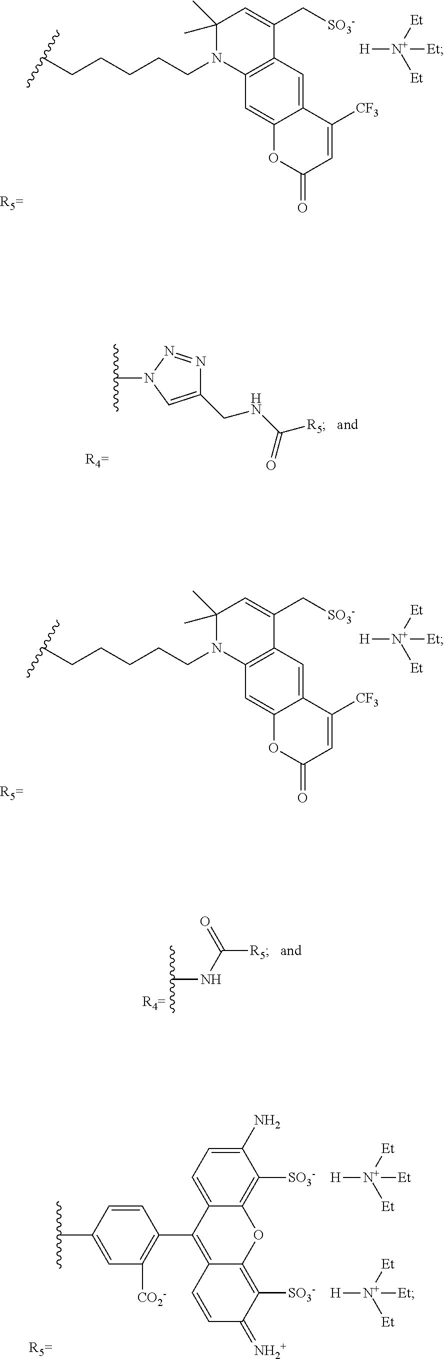

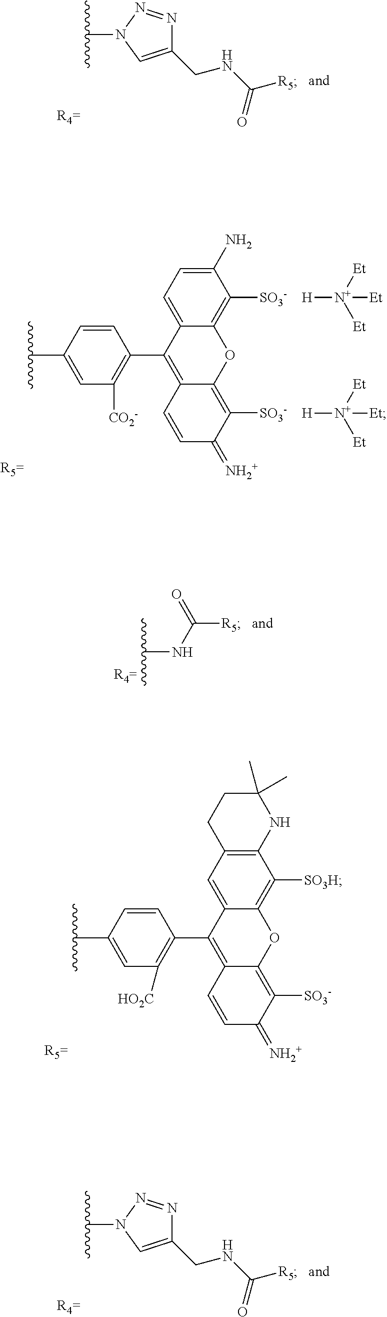

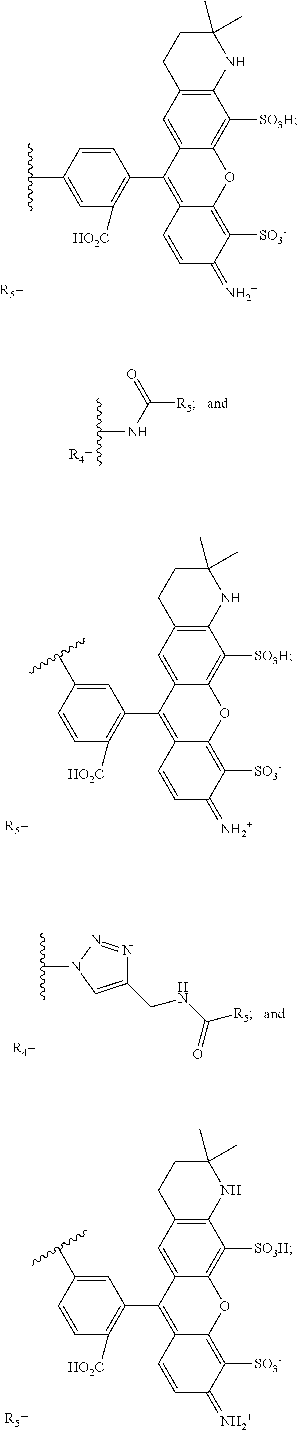

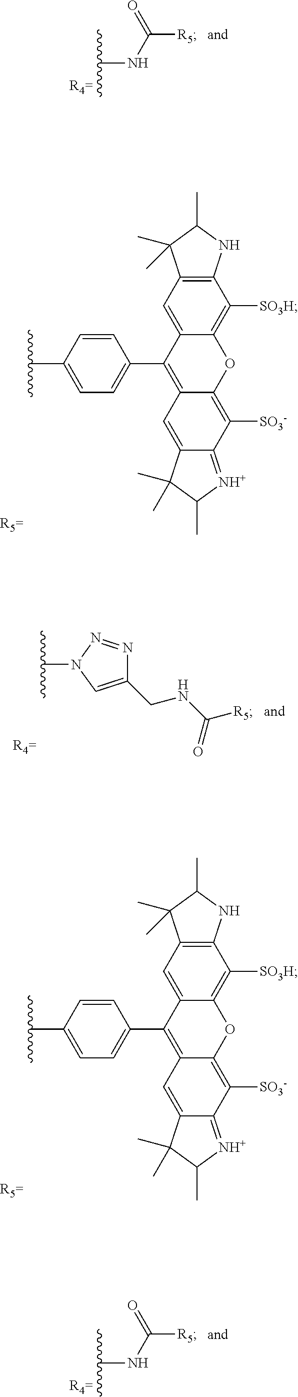

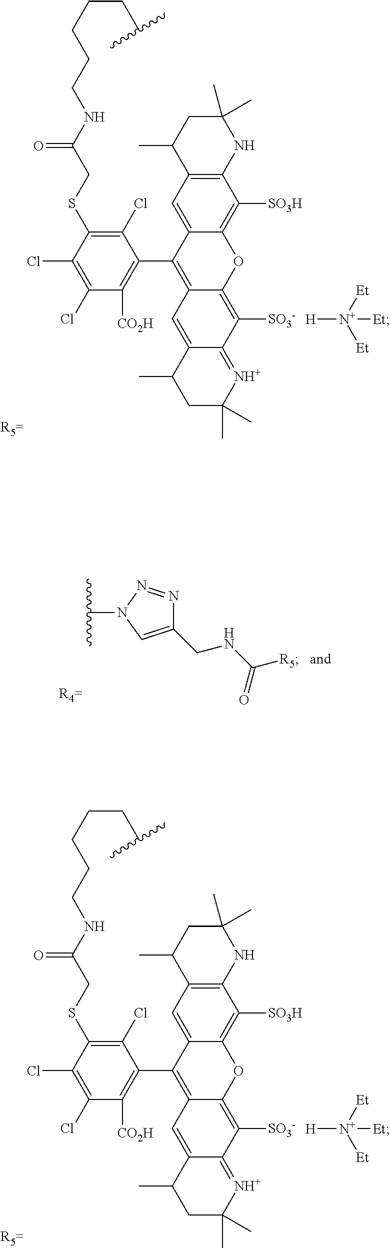

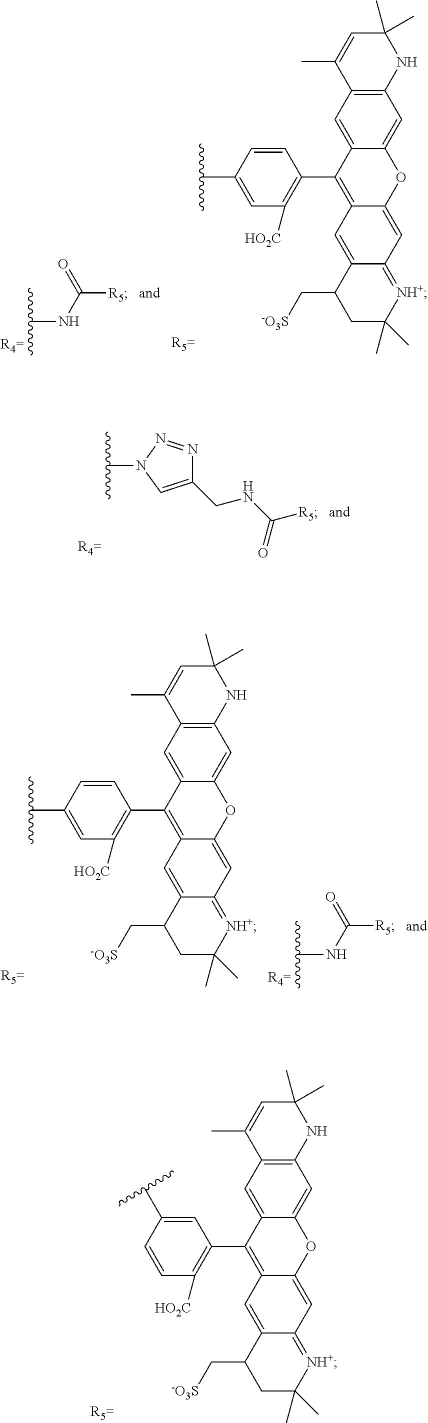

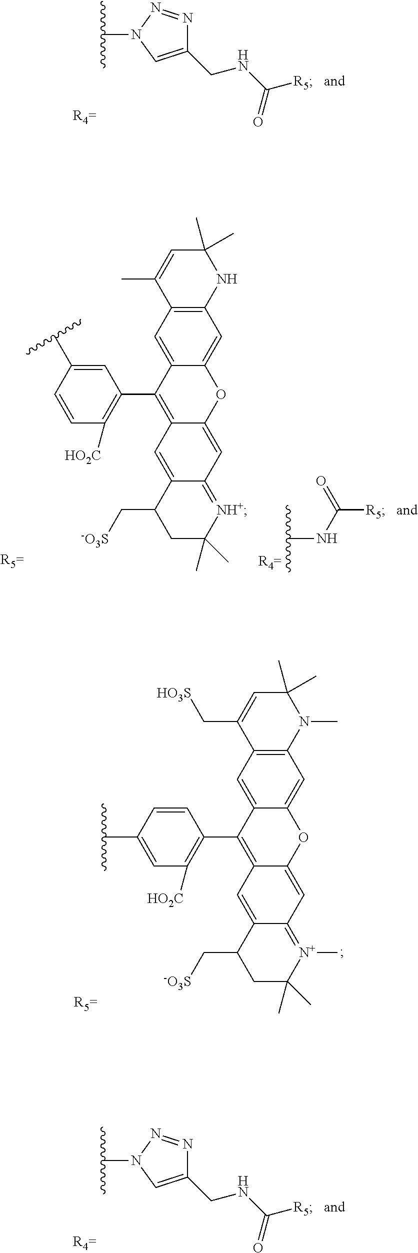

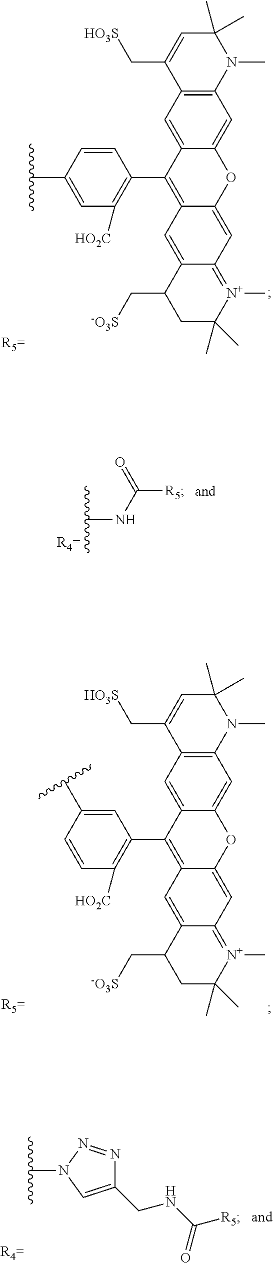

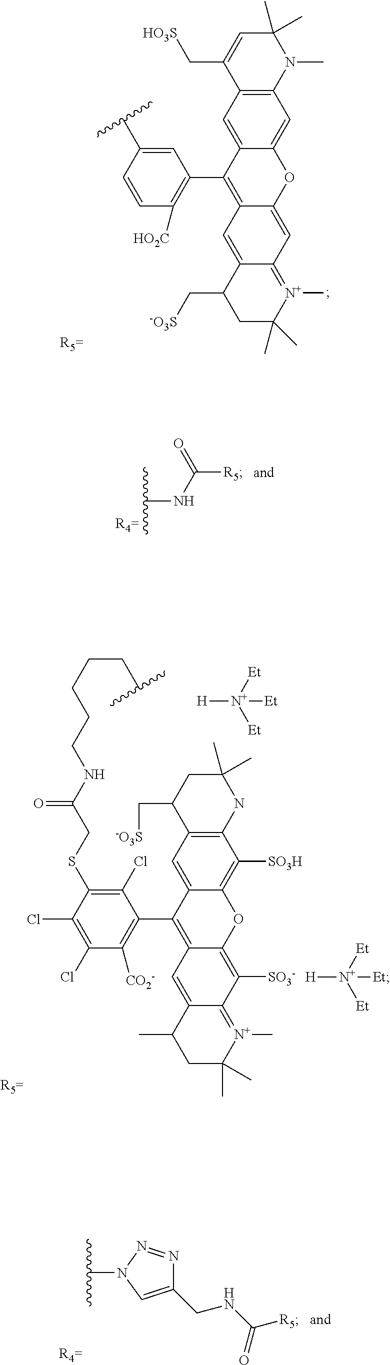

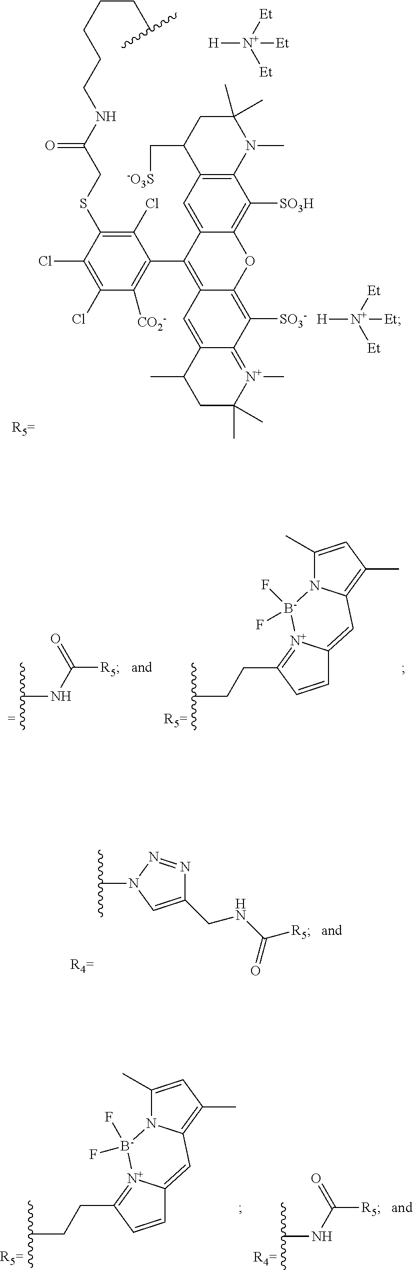

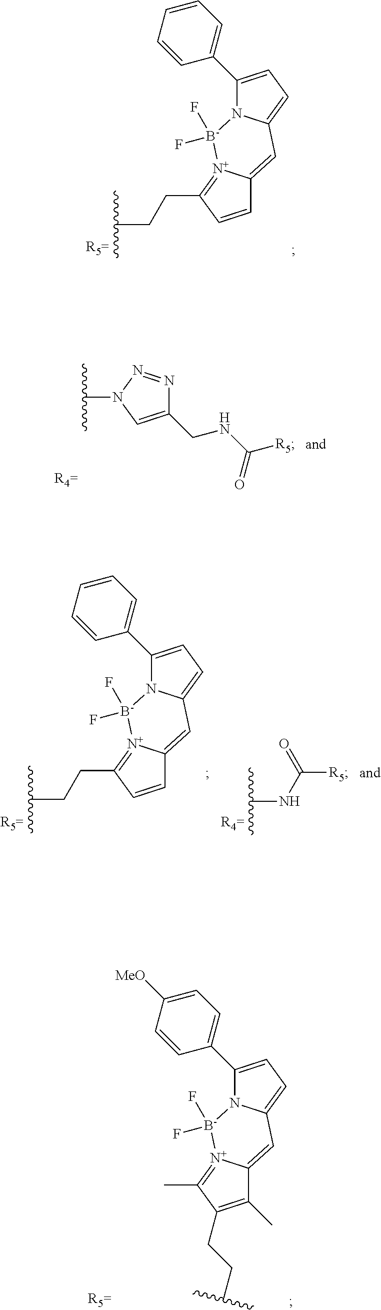

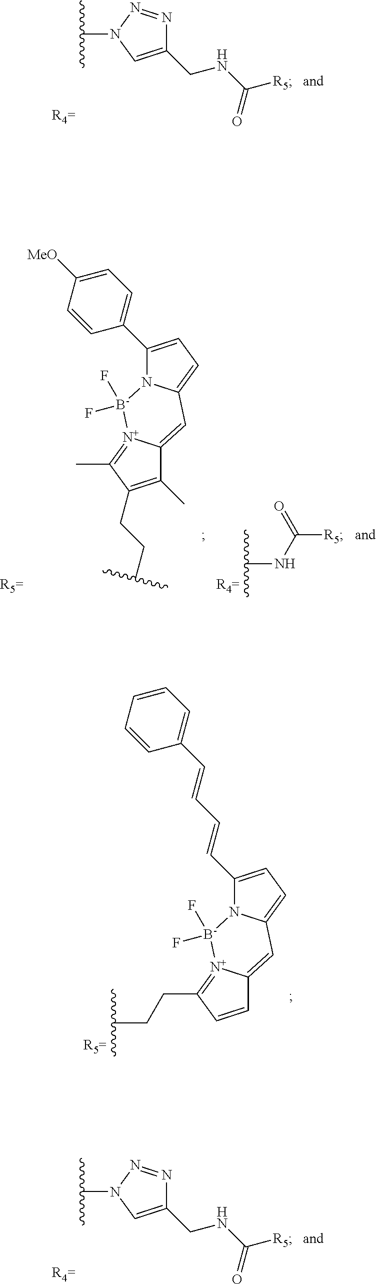

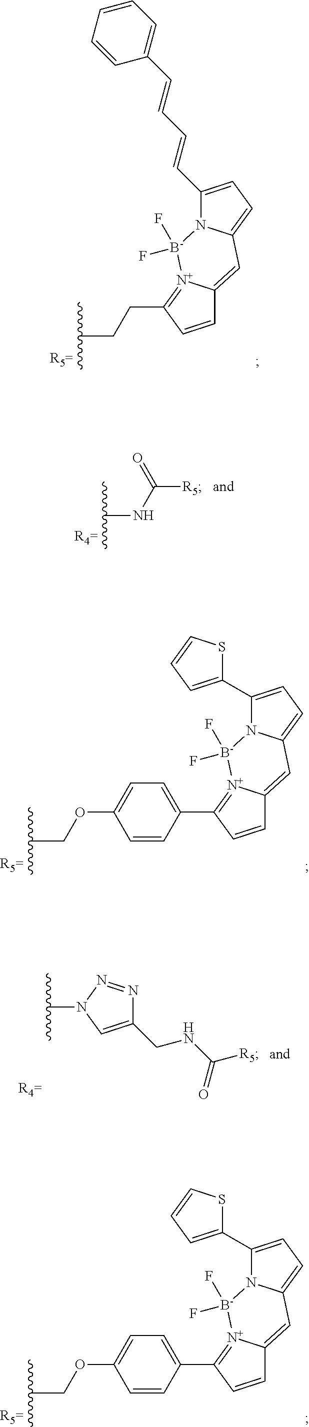

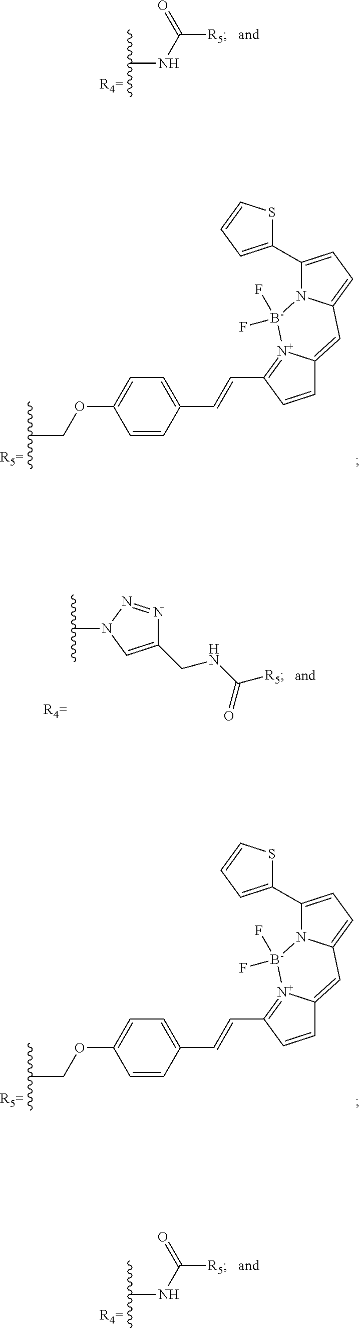

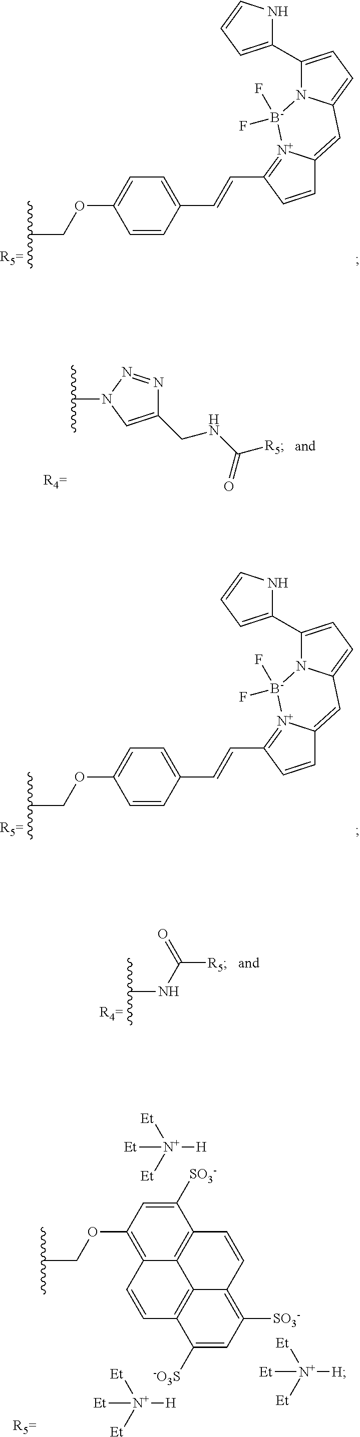

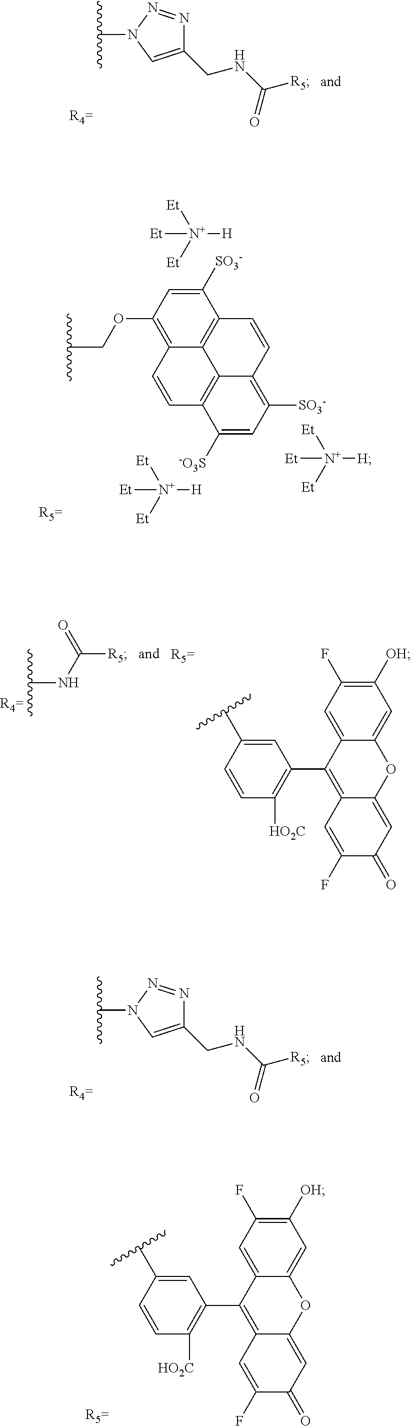

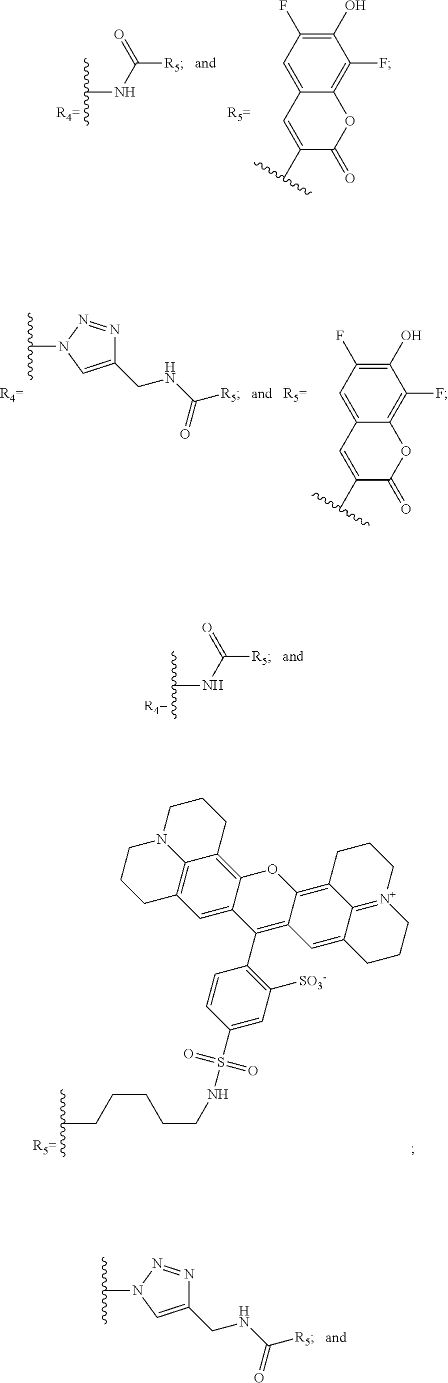

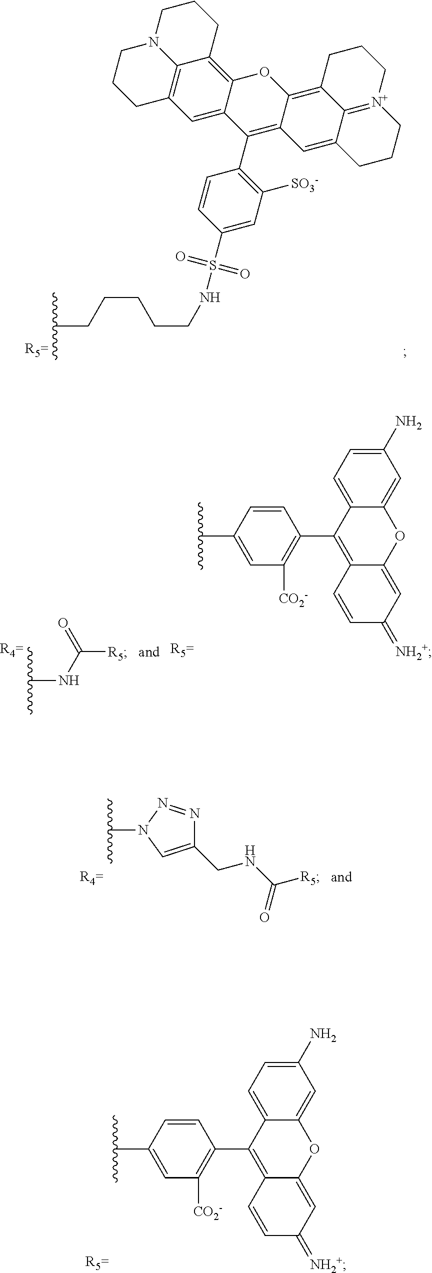

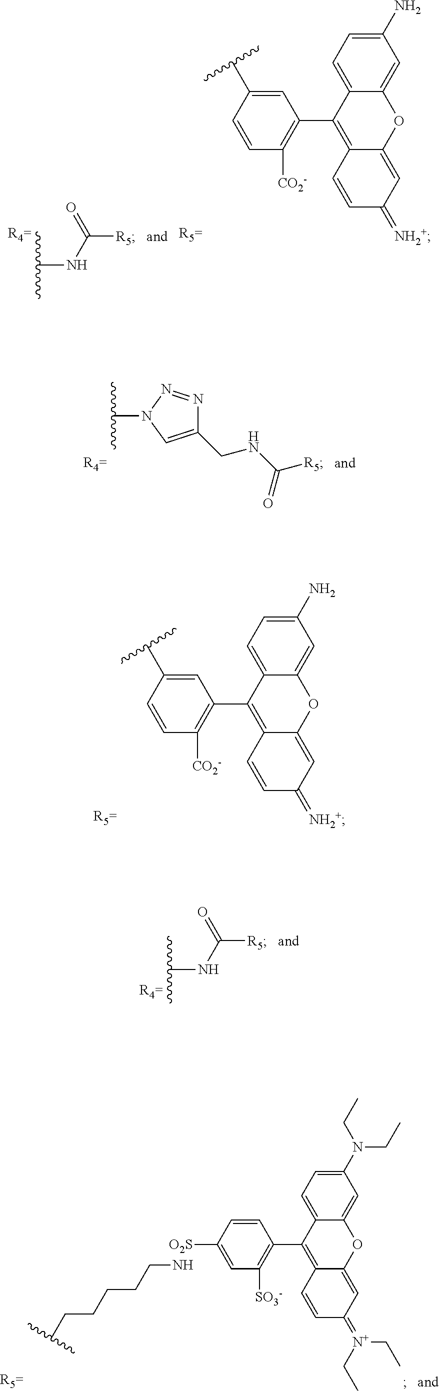

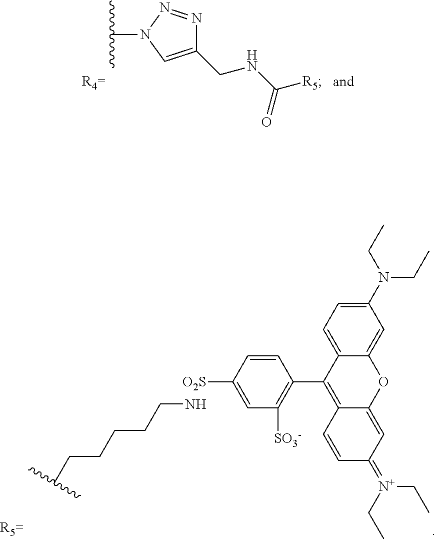

28. The compound of claim 1, according to Formula VI: 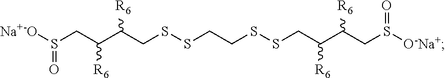
wherein, each R₆ is independently selected from 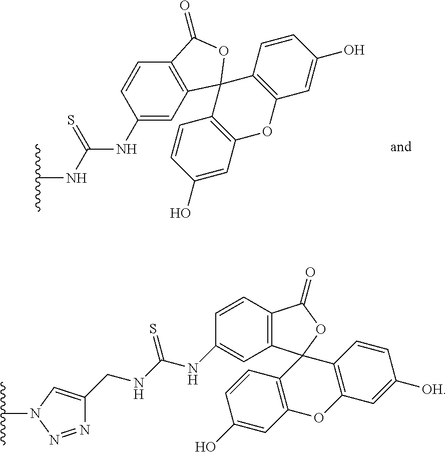 [SMILES: *NC(=S)Nc1ccc2c(c1)C1(OC2=O)c2ccc(O)cc2Oc2cc(O)ccc21 ; *n1cc(CNC(=S)Nc2ccc3c(c2)C2(OC3=O)c3ccc(O)cc3Oc3cc(O)ccc32)nn1]
